(19) United States
(12) Patent Application Publication
(10) Pub. No.: US 2026/0274799 A1
(43) Pub. Date: Sep. 17, 2026

(54) HETEROCYCLIC DERIVATIVES, COMPOSITIONS AND USES THEREOF

(71) Applicant: DANATLAS PHARMACEUTICALS CO., LTD., Beijing (CN)

(72) Inventors: Wenlai ZHOU, Beijing (CN); Jincong ZHUO, Beijing (CN)

(73) Assignee: DANATLAS PHARMACEUTICALS CO., LTD., Beijing (CN)

(21) Appl. No.: 19/669,226

(22) Filed: May 6, 2026

Related U.S. Application Data
(63) Continuation of application No. PCT/CN2024/131327, filed on Nov. 11, 2024.

(30) Foreign Application Priority Data
Nov. 10, 2023 (WO) PCT/CN2023/130913

(51) Int. Cl. C07D 215/233; A61K 31/47; A61K 31/4709; A61K 31/4745; A61K 31/497; A61P 35/00; C07D 401/10; C07D 401/12; C07D 405/10; C07D 413/10; C07D 471/04; C07D 487/04

(52) U.S. Cl. CPC C07D 215/233; A61K 31/47; A61K 31/4709; A61K 31/4745; A61K 31/497; A61P 35/00; C07D 401/10; C07D 401/12; C07D 405/10; C07D 413/10; C07D 471/04; C07D 487/04

(57) ABSTRACT

The disclosure relates to heterocyclic derivatives as shown in Formula (I), to pharmaceutical compositions comprising them, to a process for their preparation, and their use as therapeutic agents.

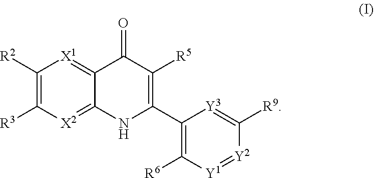

### CROSS REFERENCE OF RELATED APPLICATION

This application is continuation of PCT/CN2024/131327, filed on Nov. 11, 2024, which claims priority to PCT/CN2023/130913, entitled “HETEROCYCLIC DERIVATIVES, COMPOSITIONS AND USES THEREOF” filed on Nov. 10, 2023, all of which are incorporated by reference in their entirety.

### TECHNICAL FIELD

The present disclosure relates to heterocyclic derivatives as modulator of PPARγ. The present disclosure also relates to methods for preparing heterocyclic derivatives, pharmaceutical compositions, and their uses in the treatment of diseases or disorders, including diabetes, autoimmune diseases, inflammation, cardiovascular disease, neurodegenerative disease, especially the cancers characterized as activated function of PPARγ (gain-of-function mutation, amplification or overexpression) or RXRα gain-of-function mutation (such as S427F/Y).

### BACKGROUND

Peroxisome proliferator-activated receptor gamma (PPARγ), a member of the ligand-dependent nuclear receptor superfamily, participates in multiple physiological and pathological processes (Tianchen Peng, 2020). After binding to a specific ligand, PPARγ is activated to form an obligate heterodimer with retinoid X receptors (RXRs) and then binds to specific PPAR response elements (PPREs) of the promoter to regulate the expression of numerous downstream genes. PPARγ is mainly expressed in adipose tissue, colon, macrophage and urothelial laminae, but is practically not expressed in muscle tissue. PPARγ's most well-defined role is as a master regulator of adipogenesis, where it controls lipid metabolism, adipocyte differentiation/maintenance and insulin sensitivity (Andrew Hartley, 2022). PPARγ has also been shown to play an important role in immune regulation, tumor angiogenesis and metastasis, anti-tumor cell proliferation and tumor cell apoptosis.

Extensive studies have revealed the relationship between PPARγ and various tumors. It has been demonstrated that PPARγ affects the occurrence and progression of cancers by regulating proliferation, apoptosis, metastasis, and reactive oxygen species (ROS) and lipid metabolism (Cheng S, 2019; Cao R, 2018). Recently, PPARγ has been found to be over-expressed or genetically altered in the luminal subtype of urothelial cancer, which is consistent with the long-term use of PPARγ agonists such as TZDs (Azoulay L, 2012). Meanwhile, RXRα amplification or genetic alteration has also been found in many tumor species. It is supposed that RXRα changes, especially functional enhancement mutations such as RXRα S427F/Y, can result in stronger binding between PPARγ and RXRα proteins, leading to continuously activate downstream gene expression and ultimately promote the occurrence and progression of cancers.

Bladder cancer is one of the most prevalent malignant tumors in the urinary system (Siegel R L, 2016). Approximately 70% of newly diagnosed patients have non-muscle-invasive bladder cancer (NMJBC), and 10-20% of patients will progress to muscle-invasive bladder cancer (MJBC) (Yun S J, 2016). PPARγ gene amplification accounts for ~10% of bladder cancer tumor samples, while RXRα mutation accounts for ~5% of bladder cancer tumor samples. Research shows that pharmacologic inhibition or gene knockout of PPARγ gene inhibits the growth of a variety of urothelial cancer cells. Currently, for patients with bladder cancer, therapeutic methods are radical cystectomy and intravesical chemotherapy, but treatment options are limited with poor overall survival rates. In addition, patients with liver, breast, prostate, follicular thyroid, and other cancers are also potential candidates for PPARγ modulators. Thus, the need exists to develop effective PPARγ modulators for treating cancers.

### SUMMARY

The present disclosure relates to, inter alia, compounds of Formula (I),

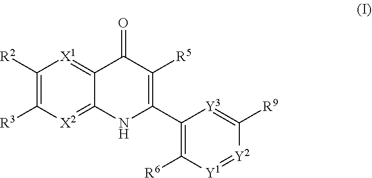

or a pharmaceutically acceptable salt, stereoisomer, solvate, N-oxide, tautomer, isotopic variant, prodrug, or deuterated compound thereof; wherein the variables are as defined below.

In another aspect, provided herein is a pharmaceutical composition comprising a compound of formula (I), or pharmaceutically acceptable salt, stereoisomer, solvate, N-oxide, tautomer, isotopic variant, prodrug, or deuterated compound thereof and at least one pharmaceutically acceptable carrier.

In another aspect, provided herein is a combination, in particular a pharmaceutical combination, comprising a compound of formula (I) of the present invention and one or more therapeutically active agents.

In another aspect, provided herein is a method of inhibiting PPARγ, comprising: contacting a PPARγ with a compound of formula (I), or a pharmaceutically acceptable salt, stereoisomer, solvate, N-oxide, tautomer, isotopic variant, prodrug, or deuterated compound thereof.

In another aspect, provided herein is a compound of formula (I) of the present invention for use as a medicament, in particular for the treatment of a disorder or disease which can be treated by inhibiting PPARγ function.

In another aspect, provided herein is a compound of formula (I) of the present invention for use in the treatment of cancer, particularly wherein the cancer is characterized as activated function of PPARγ (activating mutation, amplification or overexpression) or RXRα activating mutation (such as S427F/Y).

In another aspect, provided herein is a method of treating cancers particularly characterized as activated function of PPARγ (gain-of-function mutation, amplification or overexpression) or RXRα gain-of-function mutation (such as S427F/Y) and other diseases comprising administering to a patient a therapeutically effective amount of a compound of formula (I), or a pharmaceutically acceptable salt, stereoisomer, solvate, N-oxide, tautomer, isotopic variant, prodrug, or deuterated compound thereof.

The details of one or more embodiments are set forth in the description below. Other features, objects, and advantages will be apparent from the description and from the claims.

### DETAILED DESCRIPTION

The present disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Before the present invention is further described, it is to be understood that the invention is not limited to the particular embodiments set forth herein, and it is also to be understood that the terminology used herein is for the purpose of describing particular embodiments only, and is not intended to be limiting.

The present disclosure provides, inter alia, a compound of formula (I):

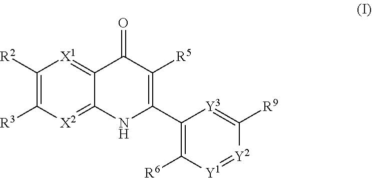

or a pharmaceutically acceptable salt, stereoisomer, solvate, N-oxide, tautomer, isotopic variant, prodrug, or deuterated compound thereof wherein:
X¹ is N or CR¹;
X² is N or CR⁴;
Y¹ is N or CR⁷;
Y² is N or CR⁸;
Y³ is N or CR¹⁰;
R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a};
R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄ alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}; and R² is not CH₃;
each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(═NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}) C(═NR^{c})NR^{c}R^{d}, NR^{d}C(═NR^{c})NR^{c}R^{d}, NR^{d}C(═NR^{c})R^{d}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂;
Cy is phenyl, C₃-C₁₀ cycloalkyl, 5-10 membered heteroaryl, 4-10 membered heterocycloalkyl; wherein, the phenyl, C₃-C₁₀ cycloalkyl, 5-10 membered heteroaryl, 4-10 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, S(O)₂NR^{c}R^{d};
Y is C₁-C₄ alkylene, C₃-C₆ alkenylene;
Z is CN, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{c}R^{D}, NR^{C}S(O)₂NR^{C}R^{D}, S(O)(═NR^{C})R^{B} or NR^{C}S(O)(═NR^{C})R^{B};
R³ is H, D, halogen, CN, C₁-C₄alkyl, C₁-C₄haloalkyl; or
R¹ and R² or R² and R³ together with the atoms to which they are attached form phenyl, C₅-C₆ cycloalkyl, 5-6 membered heteroaryl, 5-6 membered heterocycloalkyl; wherein, the phenyl, C₅-C₆ cycloalkyl, 5-6 membered heteroaryl, 5-6 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, R^{c}C(O)NR^{c}R^{d}, NR^{c}C(O)OR^{a};
R⁴ is H, D, halogen, CN;
R³ is selected from H, D, halogen, OH, CH₃, CF₃, CHF₂, CH₂F;
R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂N^{6A}R^{6B}, S(O)(=NR^{6A})R^{6B};
R^{6A} and R^{6B} are each independently selected from H, C₁-C₄ alkyl, C₁-C₄ haloalkyl, C₁-C₄ alkyl-OH, C₁-C₄ alkyl-OC₁-C₄ alkyl, C₁-C₄ alkyl-NH₂, C₁-C₄ alkyl-NHC₁-C₁ alkyl, C₁-C₄ alkyl-N(C₁-C₄ alkyl)₂, C₃-C₄ cycloalkyl optionally substituted by halogen, phenyl optionally substituted by halogen, C₁-C₄ alkyl, C₁-C₄ haloalkyl;
R⁷ and R¹⁰ are each independently selected from H, D;
R⁸ is selected from H, D, halo, OH, C₁-C₄ alkyl, C₁-C₄ haloalkyl;
R⁹ is selected from CF₃, SF₅, CN, NC, NO₂;
or R⁸ and R⁹ together with the atoms to which they are attached form 5-membered heteroaryl optionally substituted by oxo;
R^{A} and R^{a} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂;
R^{B} and R^{b} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂;
R^{C}, R^{D}, R^{c} and R^{d} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄alkyl, N(C₁-C₄alkyl)₂;
or R^{C} and R^{D} or R^{c} and R^{d} together with the N atom to which they are attached form a 4-6 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, C₁-C₄ cyanoalkyl, OC₁-C₄ alkyl, or OC₁-C₄haloalkyl;
R^{E} and R^{e} are each independently selected from H, D, C₁-C₄ alkyl, C₁-C₄ haloalkyl, or (C₁-C₄ alkoxy)-C₁-C₄ alkyl;
R^{F} and R^{f} are each independently selected from H, D, C₁-C₄ alkyl, C₁-C₄ haloalkyl, or (C₁-C₄ alkoxy)-C₁-C₄ alkyl;
R^{G}, R^{H}, R^{I}, R^{g}, R^{h} and Rⁱ are each independently selected from C₁-C₄ alkyl or phenyl.

In some embodiments, X¹ is CR¹ and X² is CR⁴.

In some embodiments, X¹ is CR¹ and X² is N.

In some embodiments, X¹ is N and X² is CR⁴.

In some embodiments, R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ is H. In some embodiments, R¹ is D.

In some embodiments, R¹ is halogen. In some embodiments, R¹ is F, Cl, Br or I. In some embodiments, R¹ is F.

In some embodiments, R¹ is CN.

In some embodiments, R¹ is C₁-C₆ alkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}. In some embodiments, R¹ is CH₃, CH₂CH₃, CH(CH₃)₂, CH₂CH₂CH₃, CH₂CH(CH₃)₂, C(CH₃)₃.

In some embodiments, R¹ is selected from C₂-C₆ alkenyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ is selected from C₂-C₆ alkynyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ is selected from C₃-C₆ cycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)N^{c}R^{d}, C(O)OR^{a}, OC(O)R^{b}, OC(O)NR^{c}R^{d}, NR^{c}R^{d}, N^{c}C(O)R^{b}, NR^{c}C(O)NR^{c}R^{d}, NR^{c}C(O)OR^{a}.

In some embodiments, R¹ is OR^{A}, and R^{A} is H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, N—C₁-C₄ alkyl, N(C₁-C₄ alkyl)₂. In some embodiments, R¹ is OH, OCH₃, OCH₂CH₃, OCH(CH₃)₂, OCH₂CH₂CH₃.

In some embodiments, R^{A} is H. In some embodiments, R^{A} is D.

In some embodiments, R^{A} is C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, R^{A} is C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, R^{A} is 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, R^{A} is phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂. In some embodiments, R^{A} is phenyl, CH₂-phenyl; each is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, R^{A} is 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, R^{A} is pyrrolyl, pyrazolyl, thiadiazolyl, pyridinyl, pyrimidinyl; each is optionally substituted with 1, 2, 3, 4 or 5 substituents independently selected from D, CN, halo, oxo, OH, NH₂, C₁-C₄alkyl, C₁-C₄ haloalkyl, OC₁-C₄alkyl, NHC₁-C₄ alkyl, N(C₁-C₄ alkyl)₂.

In some embodiments, R¹ is SR^{A}. In some embodiments, R¹ is C(O)R^{B}. In some embodiments, R¹ is C(O)NR^{C}R^{D}, In some embodiments, R¹ is C(O)OR^{A}. In some embodiments, R¹ is OC(O)R^{B}. In some embodiments, R¹ is OC(O)NR^{C}R^{D}. In some embodiments, R¹ is NR^{C}R^{D}.

In some embodiments, R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄ alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}.

In some embodiments, R² is D. In some embodiments, R² is NO₂. In some embodiments, R² is N₃. In some embodiments, R² is SF₅.

In some embodiments, R² is C₁-C₆ alkyl optionally substituted by 1, 2, 3, 4, or 5 substituents independently selected from R^{2A}, and R¹ is not CH₃.

In some embodiments, R² is CH₂CN, CH₂CH₂CN, CH(OH)CF₃, CH(OCH₃)CF₃,

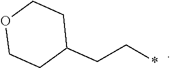 [SMILES: *CCC1CCOCC1]

In some embodiments, R² is C₂-C₆ alkenyl optionally substituted by 1, 2, 3, 4, or 5 substituents independently selected from R^{2A}. In some embodiments, R² is

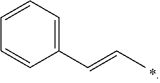

In some embodiments, R² is C₂-C₆ alkynyl optionally substituted by 1, 2, 3, 4, or 5 substituents independently selected from R^{2A}.

In some embodiments, R² is

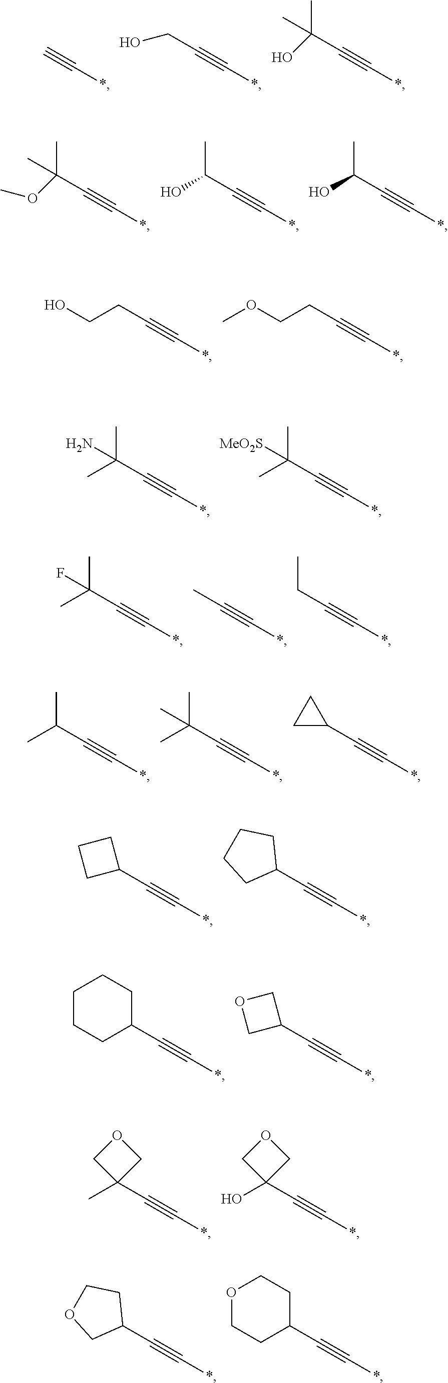

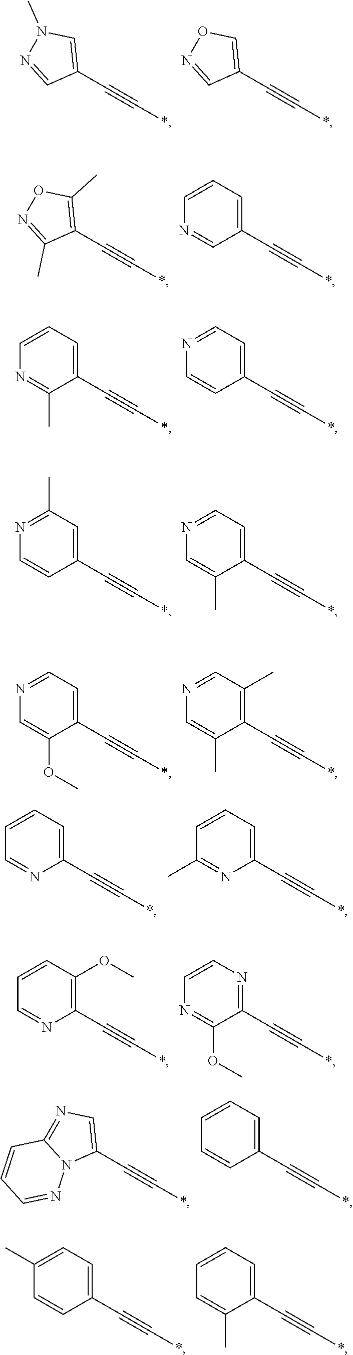

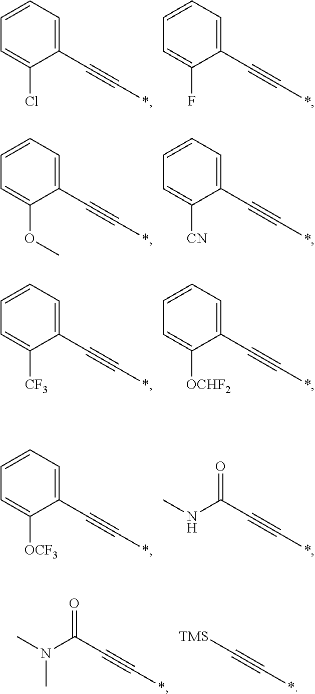

In some embodiments, R² is C₃-C₁₀ cycloalkyl optionally substituted by 1, 2, 3, 4, or 5 substituents independently selected from R^{2A}. In some embodiments, R² is cyclopropyl, cyclobutyl; each is optionally substituted by 1, 2, 3, 4, or 5 substituents independently selected from R^{2A}.

In some embodiments, R² is 4-10 membered heterocycloalkyl having a heteroatom selected from Si optionally substituted by 1, 2, 3, 4, or 5 substituents independently selected from R^{2A}.

In some embodiments, R² is O—C₀-C₄ alkyl-Cy.

In some embodiments, R² is

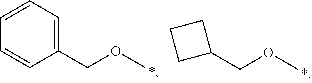

In some embodiments, R² is O—Y—Z, and Y is C₁-C₄alkylene, Z is CN, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OCR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(=NR^{C})R^{B}.

In some embodiments, R² is O—Y—Z, and Y is C₃-C₆ alkenylene, Z is CN, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(═NR^{C})R^{B}.

In some embodiments, R² is C₀-C₄ alkyl-OCy. In some embodiments, R² is C₀-C₄ alkyl-NR^{C}Cy. In some embodiments, R² is C₀-C₄ alkyl-SCy.

In some embodiments, R² is C₀-C₄ alkyl-C(O)Cy. In some embodiments, R² is C₀-C₄ alkyl-C(O)OCy. In some embodiments, R² is C₀-C₄ alkyl-C(O)NR^{C}Cy.

In some embodiments, R² is NR^{C}COCy. In some embodiments, R² is NR^{C}CO₂Cy. In some embodiments, R² is NR^{C}C(S)OCy. In some embodiments, R² is NR^{C}C(O)NR^{C}Cy. In some embodiments, R² is NR^{C}C(S)NR^{C}Cy. In some embodiments, R² is NR^{C}SO₂NR^{C}Cy.

In some embodiments, R² is C(S)Cy. In some embodiments, R² is C(S)OCy. In some embodiments, R² is C(S)NR^{C}Cy. In some embodiments, R² is NR^{C}C(S)Cy.

In some embodiments, R² is SOCy. In some embodiments, R² is SO₂Cy. In some embodiments, R² is SONR^{C}Cy.

In some embodiments, each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(═NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from H. In some embodiments, each R^{2A} is independently selected from D. In some embodiments, each R^{2A} is independently selected from halo (such as F, Cl, Br, I). In some embodiments, each R^{2A} is independently selected from CN. In some embodiments, each R^{2A} is independently selected from oxo.

In some embodiments, each R^{2A} is independently selected from C₁-C₆ alkyl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from C₂-C₆ alkenyl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{d}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from C₂-C₆ alkynyl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR'S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from C₆-C₁₀ aryl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from C₃-C₁₀ cycloalkyl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from 5-10 membered heteroaryl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from 4-10 membered heterocycloalkyl optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, each R^{2A} is independently selected from OR^{A}. In some embodiments, each R^{2A} is independently selected from SR^{A}.

In some embodiments, each R^{2A} is independently selected from C(O)R^{B}. In some embodiments, each R^{2A} is independently selected from C(O)NR^{C}R^{D}. In some embodiments, each R^{2A} is independently selected from C(O)OR^{A}. In some embodiments, each R^{2A} is independently selected from OC(O)R^{B}. In some embodiments, each R^{2A} is independently selected from OC(O)NR^{C}R^{D}.

In some embodiments, each R^{2A} is independently selected from NR^{C}R^{D}. In some embodiments, each R^{2A} is independently selected from NR^{C}C(O)R^{D}. In some embodiments, each R^{2A} is independently selected from NR^{C}C(O)NR^{C}R^{D}. In some embodiments, each R^{2A} is independently selected from NR^{C}(O)OR^{A}.

In some embodiments, each R^{2A} is independently selected from SiR^{G}R^{H}R^{I}. In some embodiments, each R^{2A} is independently selected from Si(CH₃)₃.

In some embodiments, each R^{2A} is independently selected from B(OR^{C})(OR^{D}). In some embodiments, each R^{2A} is independently selected from C(═NR^{C})NR^{C}R^{D}. In some embodiments, each R^{2A} is independently selected from NR^{D}C(═NR^{C})NR^{C}R^{D}. In some embodiments, each R^{2A} is independently selected from NR^{D}C(═NR^{c})R^{B}.

In some embodiments, each R^{2A} is independently selected from P(O)R^{E}R^{F}. In some embodiments, each R^{2A} is independently selected from P(O)OR^{E}OR^{F}. In some embodiments, each R^{2A} is independently selected from OP(O)OR^{E}OR^{F}.

In some embodiments, each R^{2A} is independently selected from S(O)RY. In some embodiments, each R^{2A} is independently selected from S(O)NR^{C}R^{D}. In some embodiments, each R^{2A} is independently selected from S(O)₂R^{B}. In some embodiments, each R^{2A} is independently selected from NR^{C}S(O)₂R^{B}. In some embodiments, each R^{2A} is independently selected from S(O)₂NR^{C}R^{D}. In some embodiments, each R^{2A} is independently selected from NR^{C}S(O)₂NR^{C}R^{D}. In some embodiments, each R^{2A} is independently selected from S(O)(═NR^{C})R^{B}. In some embodiments, each R^{2A} is independently selected from NR^{C}S(O)(=NR^{C})R^{B}.

In some embodiments, Cy is phenyl, C₃-C₁₀ cycloalkyl, 5-10 membered heteroaryl, 4-10 membered heterocycloalkyl; wherein, the phenyl, C₃-C₁₀ cycloalkyl, 5-10 membered heteroaryl, 4-10 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, S(O)₂NR^{c}R^{d}.

In some embodiments, Cy is phenyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, S(O)₂NR^{c}R^{d}.

In some embodiments, Cy is C₃-C₁₀ cycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}(O)NR^{c}R^{d}, NR^{c}C(O)OR^{a}, S(O)₂NR^{c}R^{d}.

In some embodiments, Cy is 5-10 membered heteroaryl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a} SR^{a}, C(O)R^{b}, C(O)NR^{c}R^{d}, NR^{c}R^{d}, NR^{c}(O)R^{b}, NR^{c}C(O)NR^{c}R^{d}, NR^{c}C(O)OR^{a}, S(O)₂NR^{c}R^{d}.

In some embodiments, Cy is 4-10 membered heterocycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, S(O)₂NR^{c}R^{d}.

In some embodiments, R³ is H, D, halogen, CN, C₁-C₄ alkyl, C₁-C₄ haloalkyl.

In some embodiments, R³ is H. In some embodiments, R³ is D.

In some embodiments, R³ is halogen. In some embodiments, R³ is F, Cl, Br or I. In some embodiments, R³ is F. In some embodiments, R³ is C₁.

In some embodiments, R³ is CN. In some embodiments, R³ is C₁-C₄ alkyl. In some embodiments, R³ is C₁-C₄haloalkyl.

In some embodiments, R¹ and R² together with the atoms to which they are attached form phenyl, C₅-C₆ cycloalkyl, 5-6 membered heteroaryl, 5-6 membered heterocycloalkyl; wherein, the phenyl, C₅-C₆ cycloalkyl, 5-6 membered heteroaryl, 5-6 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ and R² together with the atoms to which they are attached form phenyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ and R² together with the atoms to which they are attached form C₅-C₆ cycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ and R² together with the atoms to which they are attached form 5-6 membered heteroaryl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ and R² together with the atoms to which they are attached form pyrrolyl, imidazolyl, pyrazolyl; each is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R¹ and R² together with the atoms to which they are attached form 5-6 membered heterocycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)NR^{c}R^{d}, NR^{c}C(O)OR^{a}.

In some embodiments, R² and R³ together with the atoms to which they are attached form phenyl, C₅-C₆ cycloalkyl, 5-6 membered heteroaryl, 5-6 membered heterocycloalkyl; wherein, the phenyl, C₅-C₆ cycloalkyl, 5-6 membered heteroaryl, 5-6 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)R^{c}R^{d}, NR^{c}C(O)OR^{a}.

In some embodiments, R² and R³ together with the atoms to which they are attached form phenyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R² and R³ together with the atoms to which they are attached form C₅-C₆ cycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R² and R³ together with the atoms to which they are attached form 5-6 membered heteroaryl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R² and R³ together with the atoms to which they are attached form pyrrolyl, imidazolyl, pyrazolyl; each is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}NR^{c}C(O)NR^{c}R^{d}, NR^{c}C(O)OR^{a}. In some embodiments, R² and R together with the atoms to which they are attached form 1-methyl-1H-pyrazolyl.

In some embodiments, R² and R³ together with the atoms to which they are attached form 5-6 membered heterocycloalkyl optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

In some embodiments, R⁴ is H, D, halogen, CN.

In some embodiments, R⁴ is H. In some embodiments, R⁴ is D. In some embodiments, R⁴ is halogen. In some embodiments, R⁴ is F, Cl, Br or I. In some embodiments, R⁴ is CN.

In some embodiments, R⁵ is selected from H, D, halogen, OH, CH₃, CF₃, CHF₂, CH₂F.

In some embodiments, R⁵ is H. In some embodiments, R⁵ is D.

In some embodiments, R⁵ is halogen. In some embodiments, R⁵ is F, Cl, Br or I.

In some embodiments, R⁵ is OH. In some embodiments, R⁵ is CH₃. In some embodiments, R⁵ is CF₃. In some embodiments, R⁵ is CHF₂. In some embodiments, R⁵ is CH₂F.

In some embodiments, R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂NR^{6A}R^{6B}, S(O)(=NR^{6A})R^{6B}.

In some embodiments, R⁶ is halogen. In some embodiments, R⁶ is F, Cl, Br or I. In some embodiments, R⁶ is C₁.

In some embodiments R⁶ is OS(O)₂R^{6A}. In some embodiments, R⁶ is OS(O)₂CH₃, OS(O)₂CF₃, OTs

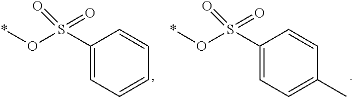

In some embodiments, R⁶ is SR^{6A}. In some embodiments, R⁶ is SCH₃.

In some embodiments, R⁶ is S(O)R^{6A}. In some embodiments, R⁶ is S(O)CH₃.

In some embodiments, R⁶ is S(O)OR^{6A}.

In some embodiments, R⁶ is S(O)₂R^{6A}. In some embodiments, R⁶ is S(O)₂CH₃, S(O)₂CH₂CH₃, S(O)₂CH(CH₃)₂, S(O)₂CH₂CH(CH₃)₂, S(O)₂CF₃, S(O)₂CH₂CF₃.

In some embodiments, R⁶ is S(O)₂OR^{6A}. In some embodiments, R is S(O)₂NR^{6A}R^{6B}.

In some embodiments, R⁶ is S(O)(═NR^{6A})R^{6B}. In some embodiments, R⁶ is S(O)(═NH)CH₃.

In some embodiments, Y¹ is N.

In some embodiments, Y¹ is CR⁷; and R⁷ is H, D. In some embodiments, Y¹ is CH.

In some embodiments, Y² is N.

In some embodiments, Y² is CR⁸.

In some embodiments, R⁸ is H. In some embodiments, R⁸ is D, In some embodiments, R⁸ is halo. In some embodiments, R⁸ is F, Cl, Br, I.

In some embodiments, R⁸ is OH. In some embodiments, R⁸ is C₁-C₄ alkyl. In some embodiments, R⁸ is C₁-C₄ haloalkyl.

In some embodiments, R⁹ is selected from CF₃, SF₅, CN, NC, NO₂.

In some embodiments, R⁹ is CF₃. In some embodiments, R⁹ is SF₅. In some embodiments, R⁹ is CN. In some embodiments, R⁹ is NC. In some embodiments, R⁹ is NO₂.

In some embodiments, R⁸ and R⁹ together with the atoms to which they are attached form 5-membered heteroaryl optionally substituted by oxo.

In some embodiments, R⁸ and R⁹ together with the atoms to which they are attached form 5-membered heteroaryl. In some embodiments, R⁸ and R⁹ together with the atoms to which they are attached form 5-membered heteroaryl oxide.

In some embodiments, R⁸ and R⁹ together with the atoms to which they are attached form

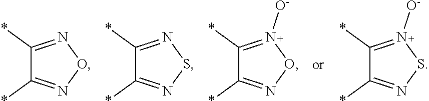

In some embodiments, Y³ is N.

In some embodiments, Y³ is CR¹⁰; and R¹⁰ is H, D. In some embodiments, Y³ is CH.

In some embodiments, each R^{A} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{A} is independently selected from H. In some embodiments, each R^{A} is independently selected from D.

In some embodiments, each R^{A} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{A} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{A} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{A} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{A} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{B} is independently selected from —I. In some embodiments, each R^{B} is independently selected from D.

In some embodiments, each R^{B} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NH C₁-C₄ alkyl, N(C₁-C₄ alkyl)₂.

In some embodiments, each R^{B} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{B} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{B} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{B} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{B} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{C} is independently selected from H. In some embodiments, each R^{C} is independently selected from D.

In some embodiments, each R^{C} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{C} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{C} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{C} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{C} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{D} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{D} is independently selected from H. In some embodiments, each R^{D} is independently selected from D.

In some embodiments, each R^{D} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{D} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{D} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{D} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{D} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄alkyl)₂.

In some embodiments, R^{C} and R^{D} together with the N atom to which they are attached form a 4-6 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄alkyl, C₁-C₄ haloalkyl, C₁-C₄ cyanoalkyl, OC₁-C₄ alkyl, or OC₁-C₄ haloalkyl.

In some embodiments, each R^{E} is independently selected from H, D, C₁-C₄ alkyl, C₁-C₄ haloalkyl, or (C₁-C₄ alkoxy)-C₁-C₄ alkyl.

In some embodiments, each R^{E} is independently selected from H. In some embodiments, each R^{E} is independently selected from D.

In some embodiments, each R^{E} is independently selected from C₁-C₄ alkyl. In some embodiments, each R^{E} is independently selected from C₁-C₄ haloalkyl. In some embodiments, each R^{E} is independently selected from (C₁-C₄ alkoxy)-C₁-C₄ alkyl.

In some embodiments, each R^{F} is independently selected from H, D, C₁-C₄ alkyl, C₁-C₄ haloalkyl, or (C₁-C₄ alkoxy)-C₁-C₄ alkyl.

In some embodiments, each R^{F} is independently selected from H. In some embodiments, each R_{F} is independently selected from D.

In some embodiments, each R_{F} is independently selected from C₁-C₄ alkyl. In some embodiments, each R_{F} is independently selected from C₁-C₄ haloalkyl. In some embodiments, each R_{F} is independently selected from (C₁-C₄ alkoxy)-C₁-C₄ alkyl.

In some embodiments, R^{G}, R^{H} and R^{I} are each independently selected from C₁-C₄ alkyl or phenyl.

In some embodiments, R^{G} is selected from C₁-C₄ alkyl or phenyl. In some embodiments, R^{G} is selected from methyl, ethyl, n-propyl, i-propyl, n-butyl, i-butyl, t-butyl, or phenyl.

In some embodiments, R^{H} is selected from C₁-C₄ alkyl or phenyl. In some embodiments, R^{H} is selected from methyl, ethyl, n-propyl, i-propyl, n-butyl, i-butyl, t-butyl, or phenyl.

In some embodiments, R^{I} is selected from C₁-C₄ alkyl or phenyl. In some embodiments, R^{I} is selected from methyl, ethyl, n-propyl, i-propyl, n-butyl, i-butyl, t-butyl, or phenyl.

In some embodiments, each R^{a} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{a} is independently selected from H. In some embodiments, each R^{a} is independently selected from D.

In some embodiments, each R^{a} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{a} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{a} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{a} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{a} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{b} is independently selected from H. In some embodiments, each R is independently selected from D.

In some embodiments, each R^{b} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{b} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{b} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{b} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{b} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄alkyl, C₁-C₄ haloalkyl, OC₁-C₄alkyl, NHC₁-C₄ alkyl, N(C₁-C₄alkyl)₂.

In some embodiments, each R^{c} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{c} is independently selected from H. In some embodiments, each R^{c} is independently selected from D.

In some embodiments, each R^{c} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄alkyl)₂.

In some embodiments, each R^{c} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄alkyl)₂.

In some embodiments, each R^{c} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{c} is independently selected from phenyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{c} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄alkyl, C₁-C₄haloalkyl, OC₁-C₄alkyl, NHC₁-C₄alkyl, N(C₁-C₄alkyl)₂.

In some embodiments, each R^{d} is independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{d} is independently selected from H. In some embodiments, each R^{d} is independently selected from D.

In some embodiments, each R^{d} is independently selected from C₁-C₆ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{d} is independently selected from C₃-C₁₀ cycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, each R^{d} is independently selected from 4-6 membered heterocycloalkyl-C₀-C₄ alkyl optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂.

In some embodiments, each R^{d} is independently selected from phenyl-C₀-C₄ alkyl optionally substituted with 1, 2, 3, 4 or 5 substituents independently selected from D, CN, halo, oxo, OH, NH₂, C₁-C₄alkyl, C₁-C₄ haloalkyl, OC₁-C₄ alkyl, NHC₁-C₄ alkyl, N(C₁-C₄alkyl)₂.

In some embodiments, each R^{d} is independently selected from 5-6 membered heteroaryl-C₀-C₄ alkyl optionally substituted with 1, 2, 3, 4 or 5 substituents independently selected from D, CN, halo, oxo, OH, NH₂, C₁-C₄ alkyl, C₁-C₄haloalkyl, OC₁-C₄ alkyl, NHC₁-C₄ alkyl, N(C₁-C₄ alkyl)₂.

In some embodiments, R^{c} and R^{d} together with the N atom to which they are attached form a 4-6 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄alkyl, C₁-C₄ haloalkyl, C₁-C₄ cyanoalkyl, OC₁-C₄ alkyl, or OC₁-C₄haloalkyl.

In some embodiments, each R^{e} is independently selected from H, D, C₁-C₄ alkyl, C₁-C₄ haloalkyl, or (C₁-C₄ alkoxy)-C₁-C₄ alkyl.

In some embodiments, each R^{e} is independently selected from H. In some embodiments, each R^{e} is independently selected from D.

In some embodiments, each R^{e} is independently selected from C₁-C₄ alkyl. In some embodiments, each R^{e} is independently selected from C₁-C₄ haloalkyl. In some embodiments, each R^{e} is independently selected from (C₁-C₄ alkoxy)-C₁-C₄ alkyl.

In some embodiments, each R^{f} is independently selected from H, D, C₁-C₄ alkyl, C₁-C₄ haloalkyl, or (C₁-C₄ alkoxy)-C₁-C₄ alkyl.

In some embodiments, each R^{f} is independently selected from H. In some embodiments, each R^{f} is independently selected from D.

In some embodiments, each R^{f} is independently selected from C₁-C₄ alkyl. In some embodiments, each R^{f} is independently selected from C₁-C₄ haloalkyl. In some embodiments, each R^{f} is independently selected from (C₁-C₄ alkoxy)-C₁-C₄ alkyl.

In some embodiments, R^{g}, R^{h} and Rⁱ are each independently selected from C₁-C₄ alkyl or phenyl.

In some embodiments, R^{g} is selected from C₁-C₄ alkyl or phenyl. In some embodiments, R^{g} is selected from methyl, ethyl, n-propyl, i-propyl, n-butyl, i-butyl, t-butyl, or phenyl.

In some embodiments, R^{h} is selected from C₁-C₄ alkyl or phenyl. In some embodiments, R^{h} is selected from methyl, ethyl, n-propyl, i-propyl, n-butyl, i-butyl, t-butyl, or phenyl.

In some embodiments, Rⁱ is selected from C₁-C₄ alkyl or phenyl. In some embodiments, Rⁱ is selected from methyl, ethyl, n-propyl, i-propyl, n-butyl, i-butyl, t-butyl, or phenyl.

In some embodiments, the compounds of Formula (I) are represented by compounds of Formula (II):

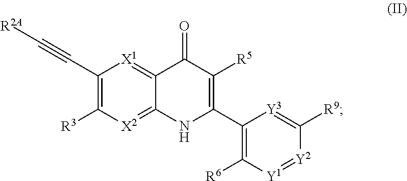

or a pharmaceutically acceptable salt, stereoisomer, solvate, N-oxide, tautomer, isotopic variant, prodrug or deuterated compound thereof;
wherein, X¹, X², Y¹, Y², Y³, R^{2A}, R⁵, R⁶, R³ and R⁹ are defined with respect to Formula (I).

In some embodiments, R^{2A} selected from H, D, CN, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, C(O)R^{B}, C(O)NR^{C}R^{D}, C(O)OR^{A}, S(O)R^{B}, S(O)NR^{C}R^{D}, S(O)₂R^{B}, S(O)₂NR^{C}R^{D}, SiR^{G}R^{H}R^{I}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{d}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}, wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, R^{2A} is selected from H, D, CN, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

In some embodiments, R^{2A} is H, D, CN, CH₃, CH₂CH₃, CH(CH₃)₂,

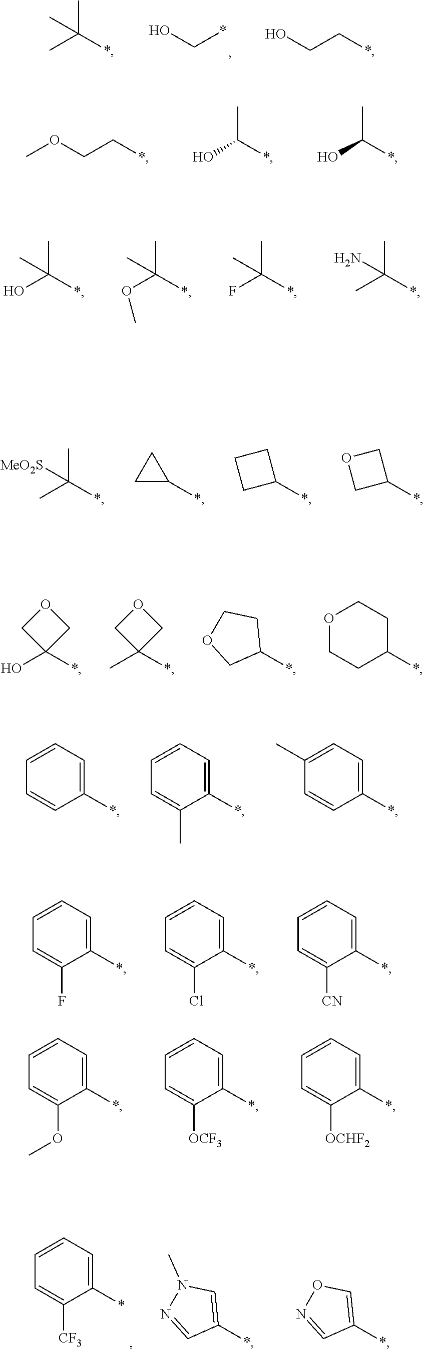

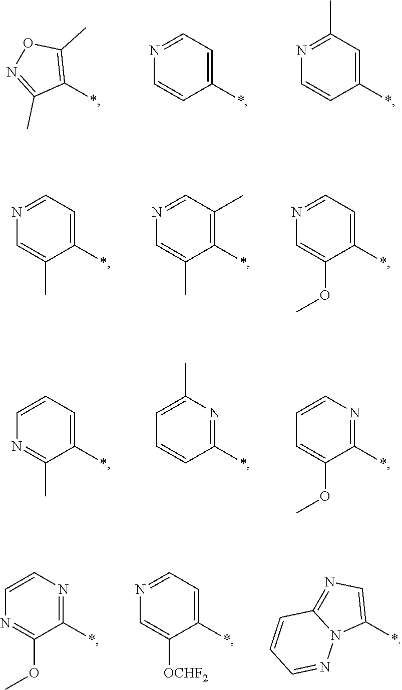

In some embodiments, the compounds of Formula (I) are the pharmaceutically acceptable salts. In some embodiments, the compounds of Formula (I) are stereoisomers. In some embodiments, the compounds of Formula (I) are solvates. In some embodiments, the compounds of Formula (I) are N-oxides of the compounds of Formula (I).

Stereoisomers of the compounds of Formula (I), and the pharmaceutical salts and solvates thereof, are also contemplated, described, and encompassed herein. Methods of using compounds of Formula (I) are described, as well as pharmaceutical compositions including the compounds of Formula (I).

In some embodiments, the compound of Formula (I) is:

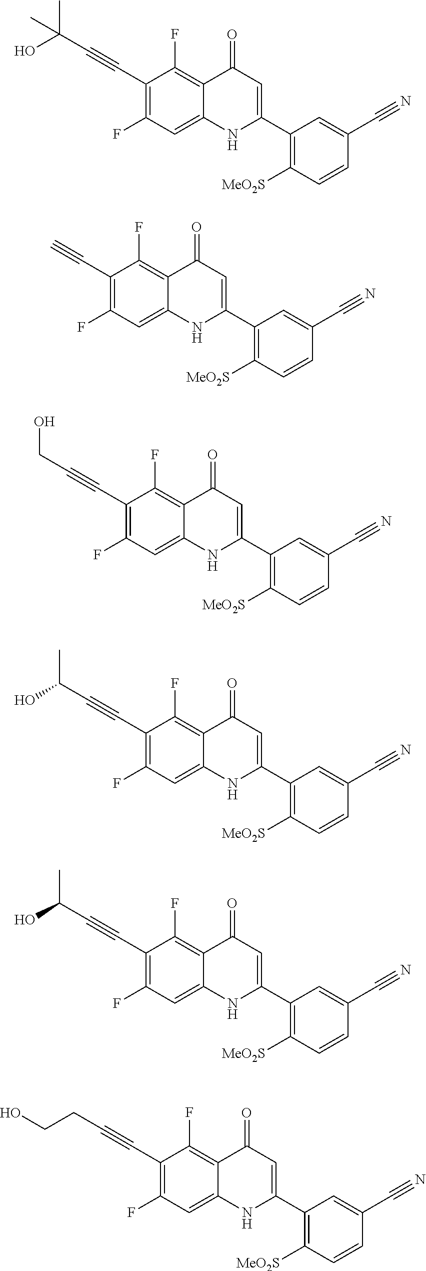 [SMILES: CC(C)(O)C#Cc1c(F)cc2[nH]c(-c3cc(C#N)ccc3S(C)(=O)=O)cc(=O)c2c1F ; C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CCO)c(F)cc2[nH]1 ; C[C@@H](O)C#Cc1c(F)cc2[nH]c(-c3cc(C#N)ccc3S(C)(=O)=O)cc(=O)c2c1F ; C[C@H](O)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CCCO)c(F)cc2[nH]1]

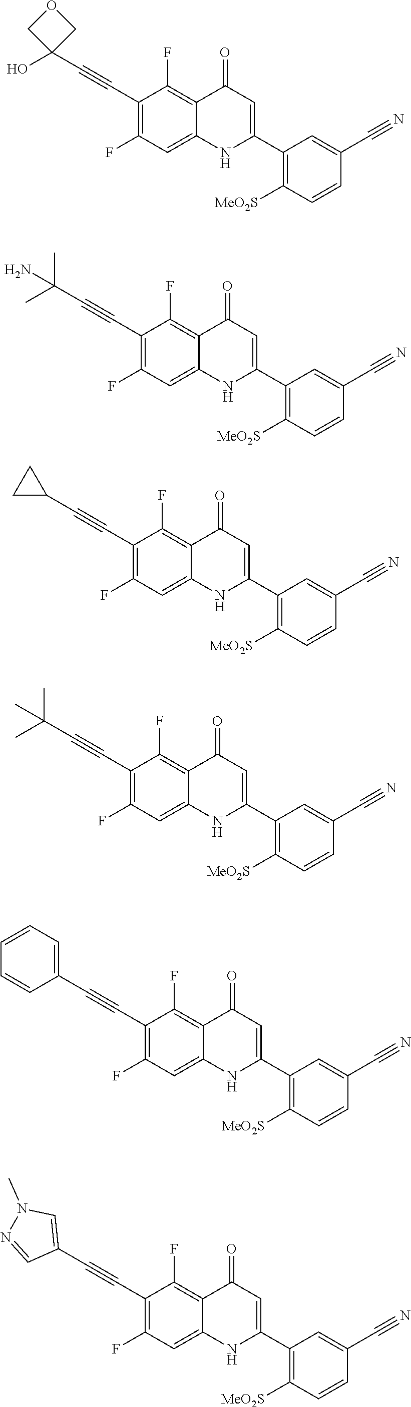 [SMILES: CS(=O)(=O)c1ccc(C#N)cc1-c1cc(=O)c2c(F)c(C#CC3(O)COC3)c(F)cc2[nH]1 ; CC(C)(N)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CC3CC3)c(F)cc2[nH]1 ; CC(C)(C)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3)c(F)cc2[nH]1 ; Cn1cc(C#Cc2c(F)cc3[nH]c(-c4cc(C#N)ccc4S(C)(=O)=O)cc(=O)c3c2F)cn1]

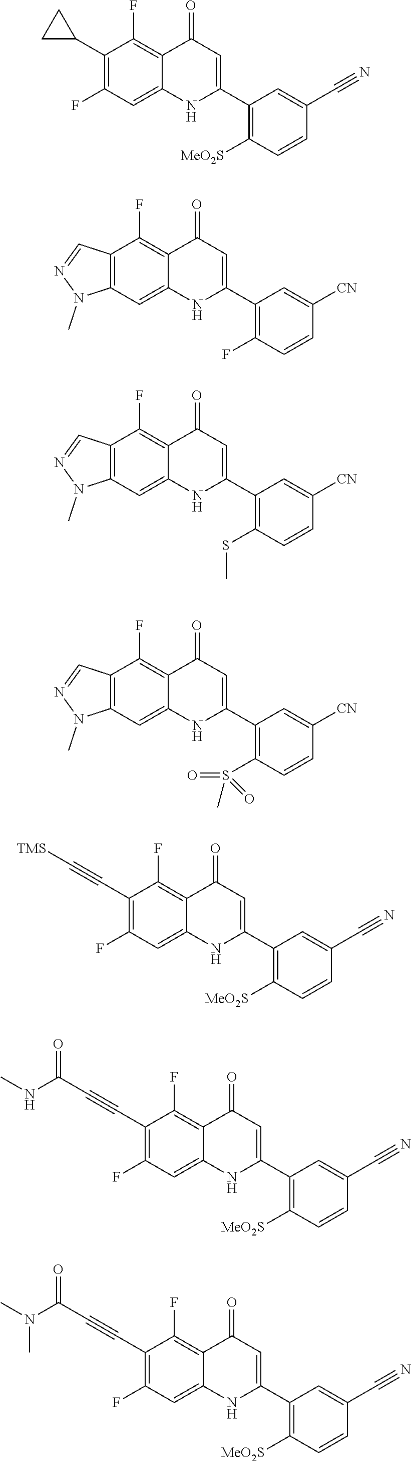

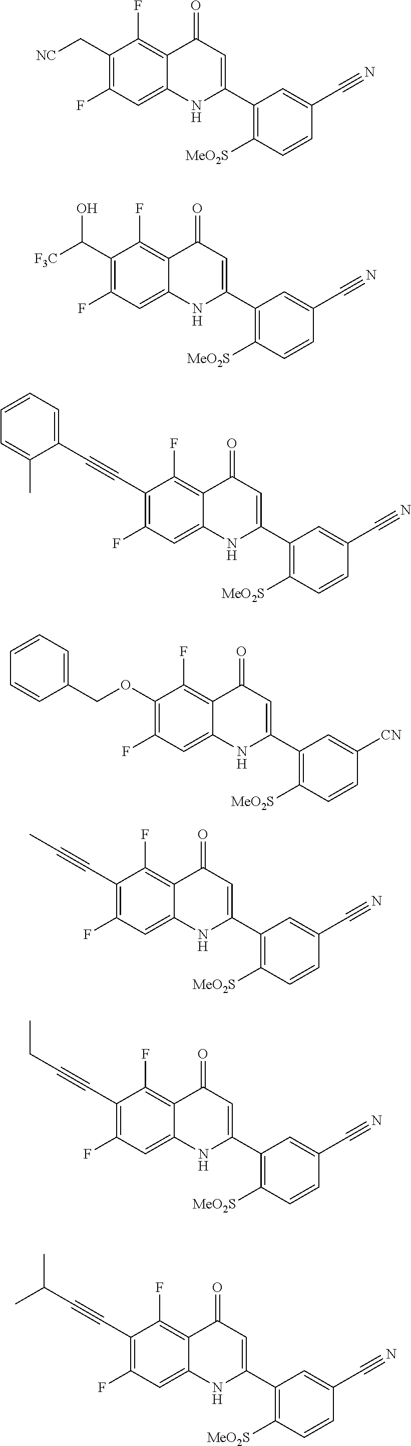

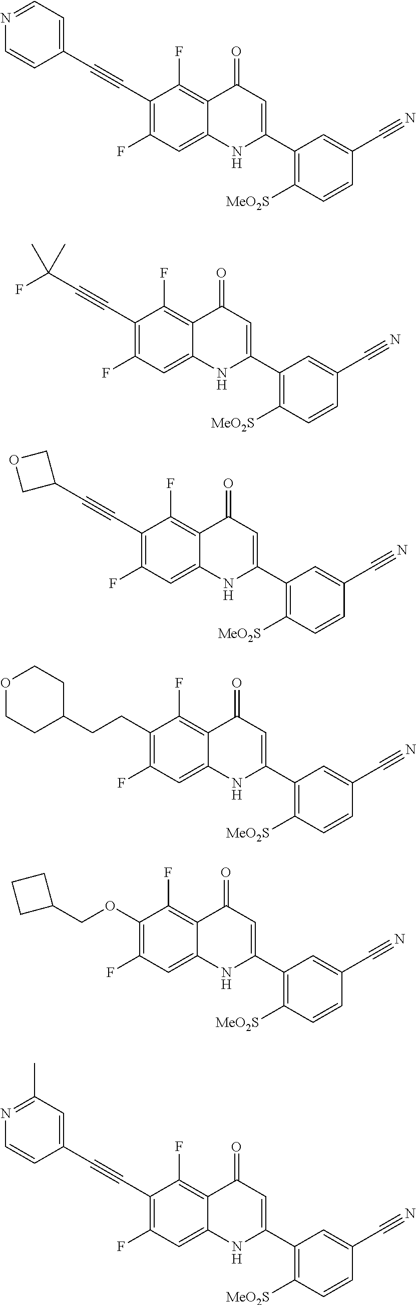 [SMILES: CS(=O)(=O)c1ccc(C#N)cc1-c1cc(=O)c2c(F)c(C#Cc3ccncc3)c(F)cc2[nH]1 ; CC(C)(F)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CC3COC3)c(F)cc2[nH]1 ; CS(=O)(=O)c1ccc(C#N)cc1-c1cc(=O)c2c(F)c(CCC3CCOCC3)c(F)cc2[nH]1 ; CS(=O)(=O)c1ccc(C#N)cc1-c1cc(=O)c2c(F)c(OCC3CCC3)c(F)cc2[nH]1 ; Cc1cc(C#Cc2c(F)cc3[nH]c(-c4cc(C#N)ccc4S(C)(=O)=O)cc(=O)c3c2F)ccn1]

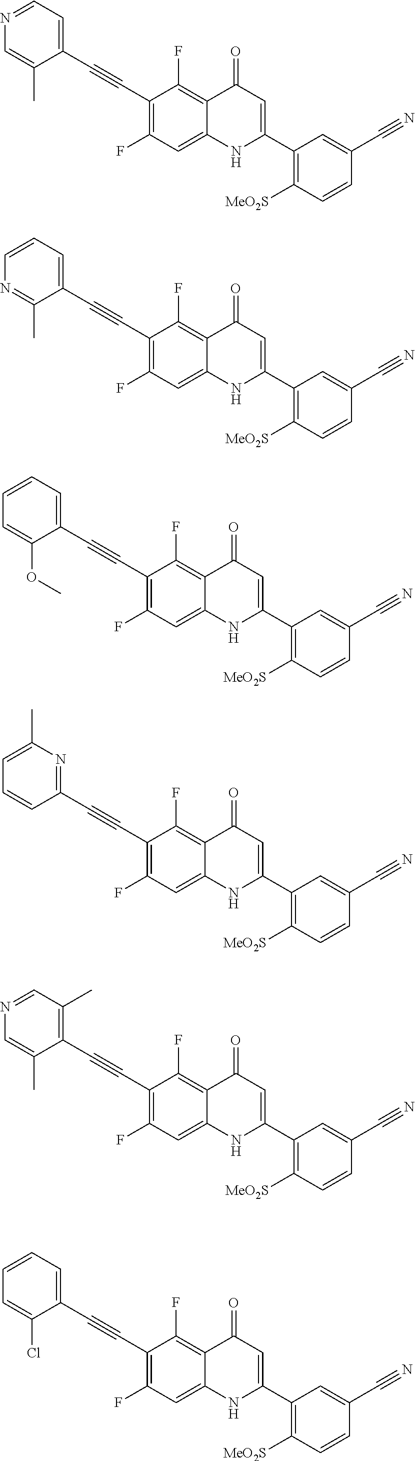 [SMILES: Cc1cnccc1C#Cc1c(F)cc2[nH]c(-c3cc(C#N)ccc3S(C)(=O)=O)cc(=O)c2c1F ; Cc1ncccc1C#Cc1c(F)cc2[nH]c(-c3cc(C#N)ccc3S(C)(=O)=O)cc(=O)c2c1F ; COc1ccccc1C#Cc1c(F)cc2[nH]c(-c3cc(C#N)ccc3S(C)(=O)=O)cc(=O)c2c1F ; Cc1cccc(C#Cc2c(F)cc3[nH]c(-c4cc(C#N)ccc4S(C)(=O)=O)cc(=O)c3c2F)n1 ; Cc1cncc(C)c1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3Cl)c(F)cc2[nH]1]

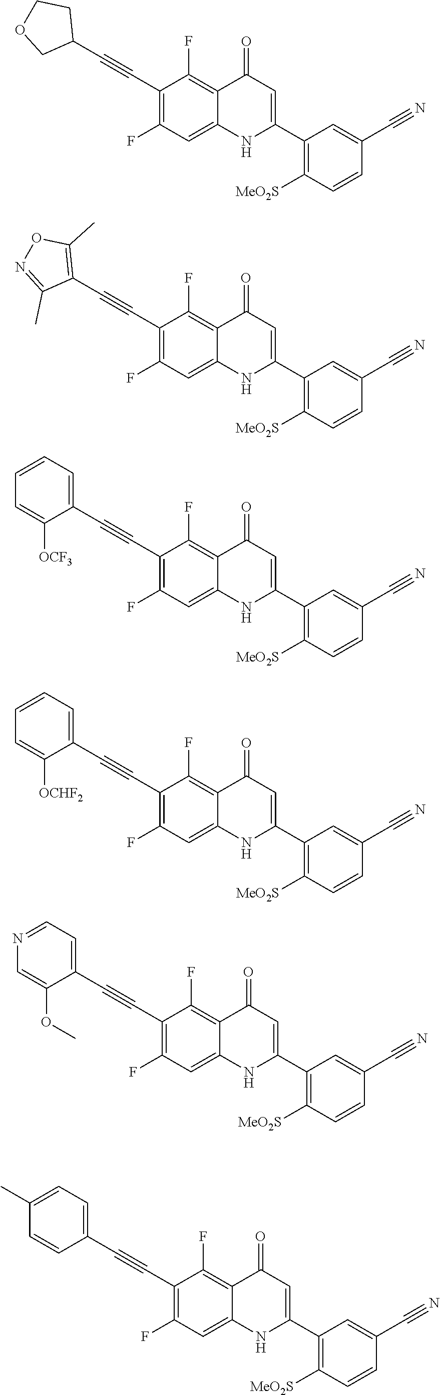 [SMILES: CS(=O)(=O)c1ccc(C#N)cc1-c1cc(=O)c2c(F)c(C#CC3CCOC3)c(F)cc2[nH]1 ; Cc1noc(C)c1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3OC(F)(F)F)c(F)cc2[nH]1 ; CS(=O)(=O)c1ccc(C#N)cc1-c1cc(=O)c2c(F)c(C#Cc3ccccc3OC(F)F)c(F)cc2[nH]1 ; COc1cnccc1C#Cc1c(F)cc2[nH]c(-c3cc(C#N)ccc3S(C)(=O)=O)cc(=O)c2c1F ; Cc1ccc(C#Cc2c(F)cc3[nH]c(-c4cc(C#N)ccc4S(C)(=O)=O)cc(=O)c3c2F)cc1]

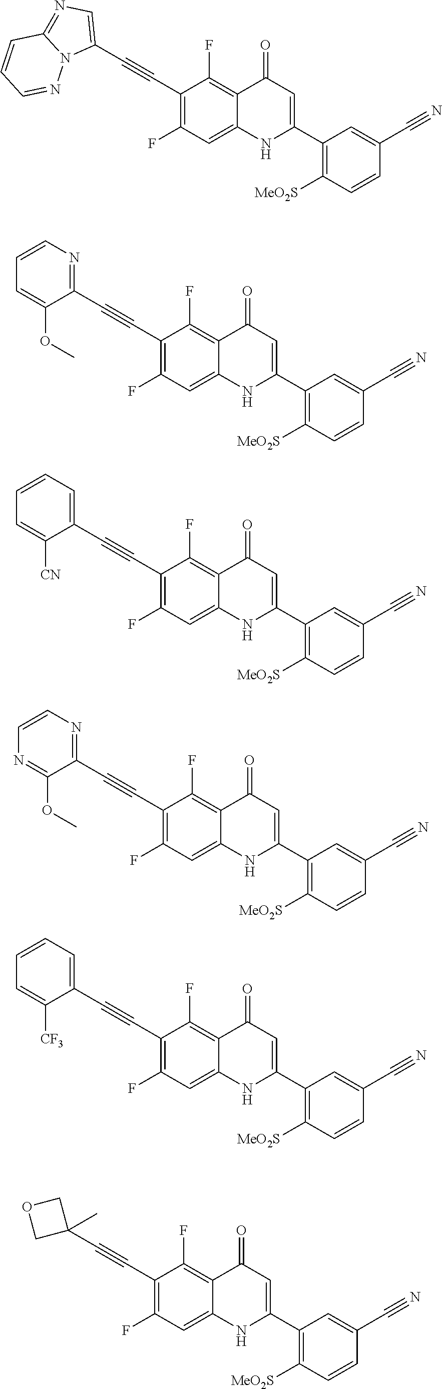 [SMILES: CS(=O)(=O)c1ccc(C#N)cc1-c1cc(=O)c2c(F)c(C#Cc3cnc4cccnn34)c(F)cc2[nH]1 ; COc1cccnc1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3C#N)c(F)cc2[nH]1 ; COc1nccnc1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3C(F)(F)F)c(F)cc2[nH]1 ; CC1(C#Cc2c(F)cc3[nH]c(-c4cc(C#N)ccc4S(C)(=O)=O)cc(=O)c3c2F)COC1]

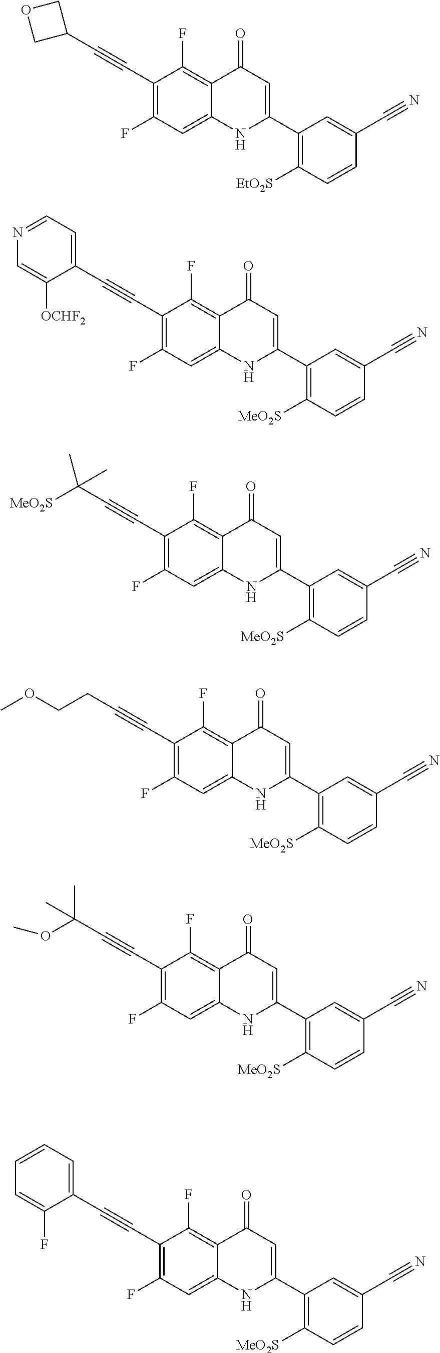

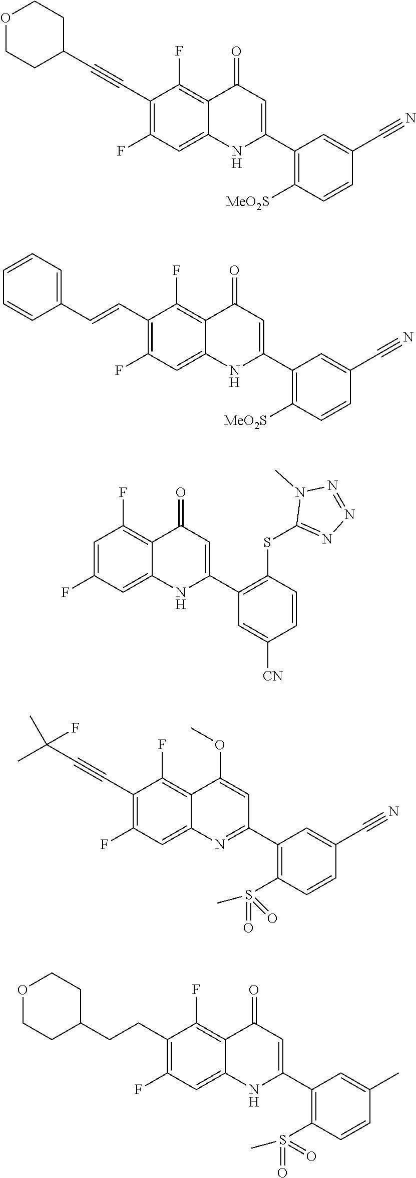 [SMILES: CS(=O)(=O)c1ccc(C#N)cc1-c1cc(=O)c2c(F)c(C#CC3CCOCC3)c(F)cc2[nH]1 ; CS(=O)(=O)c1ccc(C#N)cc1-c1cc(=O)c2c(F)c(/C=C/c3ccccc3)c(F)cc2[nH]1 ; Cn1nnnc1Sc1ccc(C#N)cc1-c1cc(=O)c2c(F)cc(F)cc2[nH]1 ; COc1cc(-c2cc(C#N)ccc2S(C)(=O)=O)nc2cc(F)c(C#CC(C)(C)F)c(F)c12 ; Cc1ccc(S(C)(=O)=O)c(-c2cc(=O)c3c(F)c(CCC4CCOCC4)c(F)cc3[nH]2)c1]

or a pharmaceutically acceptable salt thereof.

It will be apparent that the compounds of Formula (I), including all subgenera described herein, may have multiple stereogenic centers. As a result, there exist multiple stereoisomers (enantiomers and diastereomers) of the compounds of Formula (I) (and subgenera described herein). The present disclosure contemplates and encompasses each stereoisomer of any compound of Formula (I) (and subgenera described herein), as well as mixtures of said stereoisomers.

Pharmaceutically acceptable salts and solvates of the compounds of Formula (I) (including all subgenera described herein) are also within the scope of the disclosure.

Isotopic variants of the compounds of Formula (I) (including all subgenera described herein) are also contemplated by the present disclosure.

The present disclosure further provides compounds described herein, or a pharmaceutically acceptable salt thereof, for use in any of the methods described herein. The present disclosure further provides uses of a compound described herein, or a pharmaceutically acceptable salt thereof, for the preparation of a medicament for use in any of the methods described herein.

The present disclosure further provides pharmaceutical compositions comprising a compound described herein, or a pharmaceutically acceptable salt thereof and a pharmaceutically acceptable carrier.

The present disclosure further provides a method of in vitro recruit co-repressor or block co-activator to PPARγ protein, wherein, the method comprising contacting PPARγ protein with the compound disclosed herein.

The present disclosure further provides a method of modulating PPARγ in cell, which is associated with activated function of PPARγ (gain-of-function mutation, amplification or overexpression) or RXRα gain-of-function mutation (such as S427F/Y), wherein, the method comprising contacting the cell with the compound disclosed herein

The present disclosure further provides a method of treating a subject in need thereof comprising administering to the subject the compound disclosed herein, or a pharmaceutically acceptable salt thereof or the pharmaceutical composition disclosed herein.

In some embodiments, the subject is suffering from, and is in need of a treatment for, a disease or disorder having the symptom of PPARγ amplification, overexpression and gain-of-function mutation or RXRα gain-of-function mutation.

In some embodiments, the disease or disorder is a cancer.

In some embodiments, the cancer is a cancer with activated function of PPARγ (gain-of-function mutation, amplification or overexpression) or RXRα gain-of-function mutation (such as S427F/Y).

In some embodiments, the cancer is Bladder cancer.

In some embodiments, the subject is human.

Routs of administration for the compounds in the present disclosure include, but not limited to oral, injection, topical and inhalation.

### Definitions

Unless other indicated, the following terms are intended to have the meaning set forth below. Other terms are defined elsewhere throughout the specification.

As used herein, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology such as “solely”, “only” and the like in connection with the recitation of claim elements, or use of a “negative” limitation.

At various places in the present specification, variables defining divalent linking groups are described. It is specifically intended that each linking substituent include both the forward and backward forms of the linking substituent. For example, —NR(CR′R″)— includes both —NR(CR′R″)— and —(CR′R″)NR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. The phrase “optionally substituted” means unsubstituted or substituted. The term “substituted” means that a hydrogen atom is removed and replaced by a substituent. A single divalent substituent, e.g., oxo, can replace two hydrogen atoms.

As used herein, unless otherwise indicated, the term “substituted” refers to a group in which one or more hydrogen atoms are each independently replaced with the same or different substituent(s). Typical substituents include, but are not limited to, D, halo, oxo, C₁-C₆ alkyl, C₂-C₆ alkenyl, C₂-C₆ alkynyl, C₁-C₆ haloalkyl, C₁-C₆ alkyl-NR^{c}R^{d}, —(CH₂CH₂O)ₒC₁-C₆alkyl wherein o is 1-10; C₂₋₆ alkenyl-NR^{c}R^{d}, C₂₋₆ alkynyl-NR^{c}R^{d}, OC₂₋₆ alkyl-NR^{c}R^{d}, CN, NO₂, N₃, OR^{a}, SR^{a}, C(O)R^{b}, C(O)NR^{c}R^{d}, —CH₂C(O)NR^{c}R^{d}, C(O)OR^{a}, OC(O)R^{b}, OC(O)NR^{c}R^{d}, —NR^{c}R^{d}, NR^{c}C(O)R^{b}, NR^{c1}C(O)NR^{c}R^{d}, NR^{c}C(O)OR^{a}, C(═NR^{c})NR^{c}R^{d}, NR^{c}C(═R^{g1})NR^{c}R^{d}, P(R^{f})₂, P(OR^{e})₂, P(O)R^{e}R^{f}, P(O)OR^{e}OR^{f}, S(O)R^{b}, —SO(═NR^{b}), S(O)NR^{c}R^{d}, S(O)₂R^{b}, NR^{c}S(O)₂R^{b}, S(O)₂NR^{c}R^{d}, aryl, heteroaryl, spirocycloalkyl, spiroheterocycloalkyl, cycloalkyl, or heterocycloalkyl; wherein the aryl, heteroaryl, spirocycloalkyl, spiroheterocycloalkyl, cycloalkyl, or heterocycloalkyl are optionally substituted with D, halo, oxo, C₁-C₆ alkyl, C₂-C₆ alkenyl, C₂-C₆ alkynyl, C₁-C₆ haloalkyl, C₁-C₆ alkyl-NR^{c}R^{d}, C₂₋₆ alkenyl-NR^{c}R^{d}, C₂₋₆ alkynyl-NR^{c}R^{d}, OC₂₋₆ alkyl-NR^{c}R^{d}, CN, NO₂, N₃, OR^{a}, SR^{a}, C(O)R^{b}, C(O)NR^{c}R^{d}, —CH₂C(O)NR^{c}R^{d}, C(O)OR^{a}, OC(O)R^{a}, OC(O)NR^{c}R^{d}, —NR^{c}R^{d}, NR^{c}C(O)R^{b}, NR^{c}C(O)NR^{c}R^{d}, NR^{c}C(O)OR^{a}, C(═NR^{c})NR^{c}R^{d}, NR^{c}C(═NR^{c})NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}.

The term “Cn-Cm” indicates a range which includes the endpoints, wherein n and m are integers and indicate the number of carbons. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

As used herein, unless otherwise indicated, the term “alkyl”, by itself or as part of another substituent, is meant to refer to a saturated hydrocarbon group which is straight-chained or branched.

An alkyl group can contain from 1 to about 20, from 2 to about 20, from 1 to about 10, from 1 to about 8, from 1 to about 6, from 1 to about 4, or from 1 to about 3 carbon atoms. Similarly, C₁₋₈, as in C₁₋₈ alkyl is defined to identify the group as having 1, 2, 3, 4, 5, 6, 7 or 8 carbon atoms in a linear or branched arrangement. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

As used herein, unless otherwise indicated, “alkenyl” refers to an alkyl group having one or more double carbon-carbon bonds. Example alkenyl groups include, but are not limited to, ethenyl, propenyl, and the like.

As used herein, unless otherwise indicated, “alkynyl” refers to an alkyl group having one or more triple carbon-carbon bonds. Example alkynyl groups include, but are not limited to, ethynyl, propynyl, and the like.

As used herein, unless otherwise indicated, “haloalkyl” refers to an alkyl group having one or more halogen substituents. Example haloalkyl groups include, but are not limited to, CF₃, C₂F₅, CHF₂, CH₂F, CCl₃, CHCl₂, C₂Cl₅, and the like.

As used herein, unless otherwise indicated, “aryl” refers to an unsubstituted or substituted monocyclic or polycyclic (e.g., having 2, 3 or 4 fused rings) aromatic hydrocarbons. In some embodiments, aryl groups have from 6 to about 20 carbon atoms. In some embodiments, aryl groups have from 6 to about 14 carbon atoms. In some embodiments, aryl groups have from 6 to about 10 carbon atoms. Example aryl groups include, but are not limited to, phenyl, naphthyl, anthracenyl, phenanthrenyl, indanyl, indenyl, and the like.

As used herein, unless otherwise indicated, “cycloalkyl” refers to an unsubstituted or substituted non-aromatic carbocycles including cyclized alkyl, alkenyl, and alkynyl groups. Cycloalkyl groups can include mono- or polycyclic (e.g., having 2, 3 or 4 fused rings) ring systems, including fused rings, spirocyclic rings, and bridged rings (e.g., a bridged bicycloalkyl group). In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Cycloalkyl groups can be optionally substituted by oxo or sulfido (e.g., —C(O)— or —C(S)—). Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. In some embodiments, the cycloalkyl is a C₃-C₇ monocyclic cycloalkyl. In some embodiments, the cycloalkyl is a C₄-C₁₀ spirocycle or bridged cycloalkyl. Example cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. In some embodiments, cycloalkyl are cyclic-containing, non-aromatic hydrocarbon groups having from 3 to 12 carbon atoms (“C₃-C₁₂”), preferably from 3 to 6 carbon atoms (“C₃-C₆”). Examples of cycloalkyl groups include, for example, cyclopropyl (C₃; 3-membered), cyclobutyl (C₄; 4-membered), cyclopentyl (C₅), cyclohexyl (C₆), adamantanyl (C₁₀), and the like.

The term “spirocycloalkyl” when used alone or as part of a substituent group refers to a non-aromatic hydrocarbon group containing two cycloalkyl rings, and wherein the two cycloalyl rings share a single carbon atom in common.

As used herein, unless otherwise indicated, a “heteroaryl” group refers to an unsubstituted or substituted aromatic heterocycle having at least one heteroatom ring member such as boron, sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, unless otherwise indicated, “heterocycloalkyl” refers to an unsubstituted or substituted monocyclic (saturated or partially unsaturated ring) or polycyclic heterocycles having at least one non-aromatic ring (saturated or partially unsaturated ring), wherein one or more of the ring-forming carbon atoms of the heterocycloalkyl is replaced by a heteroatom selected from N, O, S and B and Si, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 3-10, 4-10, 3-7, 4-7, and 5-6 membered heterocycloalkyl groups. Heterocycloalkyl groups can also include spirocycles and bridged rings (e.g., a 5-10 membered bridged biheterocycloalkyl ring having one or more of the ring-forming carbon atoms replaced by a heteroatom independently selected from N, O, S and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members.

Example heterocycloalkyl groups include, but are not limited to, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, diazabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, oxa-azaspiro[3.5]nonanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, octahydropyrrolo[3,4-c]pyrrolyl and the like.

In some embodiments, heterocycloalkyl refers to any three to ten membered monocyclic or bicyclic, saturated ring structure containing at least one heteroatom selected from the group consisting of O, N, S, B and Si. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

In some embodiments, the term “spiroheterocycloalkyl” when used alone or as part of a substituent group refers to a non-aromatic group containing two rings, at least one of which is a heterocycloalkyl ring, and wherein the two rings share a single carbon atom in common.

As used herein, “halo” or “halogen” includes fluoro, chloro, bromo, and iodo.

As used herein, unless otherwise indicated, “alkoxy” refers to an —O-alkyl group. Example alkoxy groups include methoxy, ethoxy, propoxy (e.g., n-propoxy and isopropoxy), t-butoxy, and the like.

As used herein, unless otherwise indicated, “hydroxylalkyl” refers to an alkyl group substituted by OH.

As used herein, unless otherwise indicated, “cyanoalkyl” refers to an alkyl group substituted by CN.

As used herein, unless otherwise indicated, “alkoxyalkyl” refers to an alkyl group substituted by an alkoxy group.

As used herein, unless otherwise indicated, “haloalkoxy” refers to an —O-(haloalkyl) group.

As used herein, unless otherwise indicated, “oxo” refers to an oxygen substituent that is connected by a double bond (i.e., ═O).

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some cases, the compounds of the present disclosure may exist as rotational isomers. Descriptions of a compound of the invention that do not indicate a particular rotational isomer are intended to encompass any individual rotational isomers, as well as mixtures of rotational isomers in any proportion. Depiction of a particular rotational isomer is meant to refer to the depicted rotational isomer, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula (I) with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

As used herein, the term “inhibit”, “inhibition” or “inhibiting” refers to the reduction or suppression of a given condition, symptom, or disorder, or disease, or a significant decrease in the baseline activity of a biological activity or process.

The term “modulator” refers to agents that bind to the receptor binding site as an agonist, antagonist or inverse agonist to increase or inhibit the activity of receptor. The term “inverse agonist” refers to agents that bind to the same receptor binding site as an agonist (e.g., the binding site of a nuclear receptor such as PPARγ) and not only antagonizes the effects of an agonist but, moreover, exerts the opposite effect by suppressing spontaneous receptor signaling (when present). “PPARγ inverse agonist” as used herein means a compound that increase the repressive state (co-repressor recruitment) and inhibit active state (co-activator blockade).

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to bladder cancer, prostate cancer, gastric cancer, lung cancer, melanoma, endometrial cancer, sarcoma, breast cancer, hepatobiliary cancer, adrenocortical carcinoma, colorectal cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “PPARγ” as used herein refers to the protein of Peroxisome proliferator-activated receptor gamma, which is divided into two PPARγ isoforms, γ1 and γ2, derived from separate transcriptional start sites. γ1 and γ2 are identical except for an additional 30 amino acids at the N terminus of PPARγ2. The term “PPARγ” includes mutants, fragments, variants, isoforms, and homologs of full-length wild-type PPARγ. In one embodiment, the protein is encoded by the PPARγ gene (Entrez gene ID 5468; Ensembl ID ENSG00000132170). Exemplary PPARγ sequences are available at the Uniprot database under accession number P37231.

The term “RXRα” as used herein refers to the protein of retinoid X receptor alpha, which is an important therapeutic target impacting diverse biological processes In one embodiment, the protein is encoded by the RXRα gene (Entrez gene ID 6256; Ensembl ID ENSG00000186350). Exemplary RXRα sequences are available at the Uniprot database under accession number P19793.

“Disease or disorder mediated by PPARγ” includes a disease or condition, such as cancer, which is treated by increasing the repressive state (such as co-repressor SMRT recruitment) of PPARγ to a higher degree. In particular, this can include cancers characterized as activated function of PPARγ (gain-of-function mutation, amplification or overexpression) or RXRα gain-of-function mutation (such as S427F/Y).

“Compounds of the present disclosure,” and equivalent expressions, are meant to embrace compounds of Formula (I) as described herein, as well as its subgenera, which expression includes the stereoisomers (e.g., enantiomers, diastereomers) and constitutional isomers (e.g., tautomers) of compounds of Formula (I) as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

### Pharmaceutical Compositions

Also provided are pharmaceutical compositions comprising compounds of Formula (I), or a pharmaceutically acceptable salt, stereoisomer, solvate, N-oxide, tautomer, isotopic variant, prodrug, or deuterated compound thereof, and a pharmaceutically acceptable carrier.

The compositions may be in a form suitable for oral use (for example as tablets, lozenges, hard or soft capsules, aqueous or oily suspensions, emulsions, dispersible powders or granules, syrups or elixirs), for injection use (for example as aqueous or oil suspensions, or emulsions, with sesame oil, corn oil, cottonseed oil, or peanut oil, as well as elixirs, mannitol, dextrose, or a sterile aqueous solution, and similar pharmaceutical vehicles), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions may be obtained by conventional procedures using conventional pharmaceutical excipients, well known in the art. Thus, compositions intended for oral use may contain, for example, one or more coloring, sweetening, flavoring and/or preservative agents.

An effective amount of a compound of Formula (I) or a pharmaceutically salt thereof for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.1 mg to 1000 mg of Formula (I) or a pharmaceutically salt thereof with an appropriate and convenient amount of excipients which may vary from about 5 to about 98 percent by weight of the total composition.

The size of the dose for therapeutic or prophylactic purposes of a compound of the Formula (I) will naturally vary according to the nature and severity of the conditions, the age and sex of the animal or patient and the route of administration, according to well-known principles of medicine.

Described below are non-limiting exemplary pharmaceutical compositions and methods for preparing the same.

### Methods of Administration

The compounds of Formula (I) or a pharmaceutically salt thereof or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC₅₀).

As used herein, the term “EC₅₀” refers to the half maximal effective concentration of an activator in activating biological or biochemical function. This quantitative measure indicates how much of a particular activator is needed to activate a given biological process (or component of a process, i.e. an enzyme, cell, cell receptor or microorganism) by half. In other words, it is the half maximal (50%) effective concentration (EC) of a substance (50% EC, or EC₅₀).

In some embodiments, the subject methods utilize PPARγ inverse agonist/antagonist to increase PPARγ repressive state (such as co-repressor SMRT recruitment) with an EC₅₀ value of about or less than a predetermined value or decrease PPARγ activating state (such as co-activator DRIP205 blockade) with an IC₅₀ value of about or less than a predetermined value, as ascertained in an in vitro assay. In other embodiments, the subject methods utilize PPARγ agonist to decrease PPARγ repressive state (such as co-repressor SMRT blockade) with an IC₅₀ value of about or less than a predetermined value or increase PPARγ activating state (such as co-activator DRIP205 recruitment) with an EC₅₀ value of about or less than a predetermined value, as ascertained in an in vitro assay. In these embodiments, the PPARγ inverse agonist, antagonist or agonist acts on PPARγ activity with an EC₅₀ or IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the disease or disorder associated with PPARγ is cancer. In some embodiments, the cancer is associated with an tip-regulated peroxisome proliferator-activated receptor (PPAR) signaling pathway. In some aspects, the up-regulated PPAR signaling pathway is associated with increased expression of one or more genes selected from Uroplakin 1A (LTPKIA), Uroplakin IB (UPK1B), Uroplakin (UPK2), Keratin 20 (KRT20), GATA Binding Protein 3 (GAT A3), Nuclear Receptor Corepressor 1 (NCOR1), Nuclear Receptor Corepressor 2 (NCOR2), Fatty Acid Binding Protein 4 (FABP4), Forkhead Box A1 (FOXAl), CD36 Molecule (CD36), Acyl-CoA Oxidase 1 (ACOX1), 3-Hydroxy-3-Methylglutaryl-CoA Synthase 2 (HMGCS2), Acyl-CoA Synthetase Long-Chain Family Member 5 (ACSL5), Arachidonate 5-Lipoxygenase (ALOX5), Acyl-CoA Synthetase Long-Chain Family Member 1 (ACSL1), and Angiopoietin Like 4 (ANGPTL4), Liver X Receptor Alpha (LXRA), Cell Death Inducing DFFA Like Effector C (CIDEC), Phosphoenolpyruvate carboxykinase (PEPCK), Aquaporin 7 (AQP7), etc.

In other embodiments, said method is for treating a disease or cancer selected from the cancers with poor chemotherapy response or chemotherapy resistance, e.g., intravesical chemotherapy of bladder.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other methods, compounds of the disclosure, as well as pharmaceutical compositions comprising them, can be administered to treat any of the described diseases, alone or in combination with one or more other agents.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith el ah, March's Advanced Organic Chemistry: Reactions, Mechanisms, and Structure, 8th Ed. (Wiley, 2019); Peturssion et al, “Protecting Groups in Carbohydrate Chemistry,” J Chem. Educ., 1997, 74(11), 1297; and Wuts et al., Protective Groups in Organic Synthesis, 5th Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature”, “room temperature”, and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The present disclosure provides the following embodiments:
1. A compound of formula (I), or a pharmaceutically acceptable salt, stereoisomer, solvate, N-oxide, tautomer, isotopic variant, prodrug, or deuterated compound thereof, wherein:

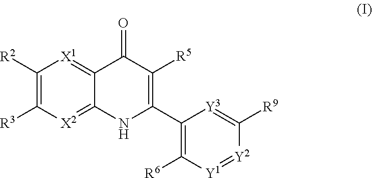

or a pharmaceutically acceptable salt, stereoisomer, solvate, N-oxide, tautomer, isotopic variant, prodrug, or deuterated compound thereof, wherein:
   X¹ is N or CR¹;
   X² is N or CR⁴;
   Y¹ is N or CR⁷;
   Y² is N or CR⁸;
   Y³ is N or CR¹⁰;
R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D}, C(O)OR^{A}, OC(O)R^{A}, OC(O)NR^{C}R^{D}, NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a};
R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄ alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}; and R² is not CH₃;
each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(═NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂;
Cy is phenyl, C₃-C₁₀ cycloalkyl, 5-10 membered heteroaryl, 4-10 membered heterocycloalkyl; wherein, the phenyl, C₃-C₁₀ cycloalkyl, 5-10 membered heteroaryl, 4-10 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, NR^{c}R^{d}, NR^{c}C(O)R^{b}, NR^{c}C(O)NR^{c}R^{d}, NR^{c}(O)OR^{a}, S(O)₂NR^{c}R^{d},
Y is C₁-C₄ alkylene, C₃-C₆ alkenylene;
Z is CN, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(=NR^{C})R^{B};
R³ is H, D, halogen, CN, C₁-C₄alkyl, C₁-C₄haloalkyl; or
R¹ and R² or R² and R³ together with the atoms to which they are attached form phenyl, C₅-C₆ cycloalkyl, 5-6 membered heteroaryl, 5-6 membered heterocycloalkyl; wherein, the phenyl, C₅-C₆ cycloalkyl, 5-6 membered heteroaryl, 5-6 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a};
R⁴ is H, D, halogen, CN;
R⁵ is selected from H, D, halogen, OH, CH₃, CF₃, CHF₂, CH₂F;
R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂NR^{6A}R^{6B}, S(O)(=NR^{6A})R^{6B};
R^{6A} and R^{6B} are each independently selected from H, C₁-C₄ alkyl, C₁-C₄ haloalkyl, C₁-C₄ alkyl-OH, C₁-C₄ alkyl-OC₁-C₄ alkyl, C₁-C₄ alkyl-NH₂, C₁-C₄ alkyl-NHC₁-C₄ alkyl, C₁-C₄ alkyl-N(C₁-C₄ alkyl)₂, C₃-C₄ cycloalkyl optionally substituted by halogen, phenyl optionally substituted by C₁-C₄ alkyl, C₁-C₄ haloalkyl;
R⁷ and R¹⁰ are each independently selected from H, D;
R⁸ is selected from H, D, halo, OH, C₁-C₄ alkyl, C₁-C₄ haloalkyl;
R⁹ is selected from CF₃, SF₅, CN, NC, NO₂;
or R⁸ and R⁹ together with the atoms to which they are attached form 5-membered heteroaryl optionally substituted by oxo;
R^{A} and R^{a} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂;
R^{B} and R^{b} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄alkyl, C₁-C₄ haloalkyl, OC₁-C₄ alkyl, NHC₁-C₄ alkyl, N(C₁-C₄ alkyl)₂;
R^{C}, R^{D}, R^{c} and R^{d} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂;
or R^{C} and R^{D} or R^{c} and R^{d} together with the N atom to which they are attached form a 4-6 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, C₁-C₄ cyanoalkyl, OC₁-C₄ alkyl, or OC₁-C₄ haloalkyl;
R^{E} and R^{e} are each independently selected from H, D, C₁-C₄ alkyl, C₁-C₄ haloalkyl, or (C₁-C₄ alkoxy)-C₁-C₄ alkyl;
R^{F} and R^{f} are each independently selected from H, D, C₁-C₄ alkyl, C₁-C₄ haloalkyl, or (C₁-C₄ alkoxy)-C₁-C₄ alkyl;
R^{G}, R^{H}, R^{I}, R^{g}, R^{h} and Rⁱ are each independently selected from C₁-C₄ alkyl or phenyl.
2. The compound of embodiment 1, wherein, X¹ is CR¹ and X² is CR⁴; X¹ is CR¹ and X² is N; X¹ is N and X² is CR⁴.
3. The compound of embodiment 1 or 2, wherein, R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D} C(O)OR^{A}, OC(O)R^{B}, OC(O)NR^{C}R^{D}, NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)NR^{c}R^{d}, NR^{c}C(O)OR^{a}.
4. The compound of anyone of embodiment 1-3, wherein, R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄ alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}; preferably, R² is selected from D, NO₂, N₃, SF₅, CH₂CN, CH₂CH₂CN, CH(OH)CF₃, CH(OCH₃)CF₃, cyclopropyl,

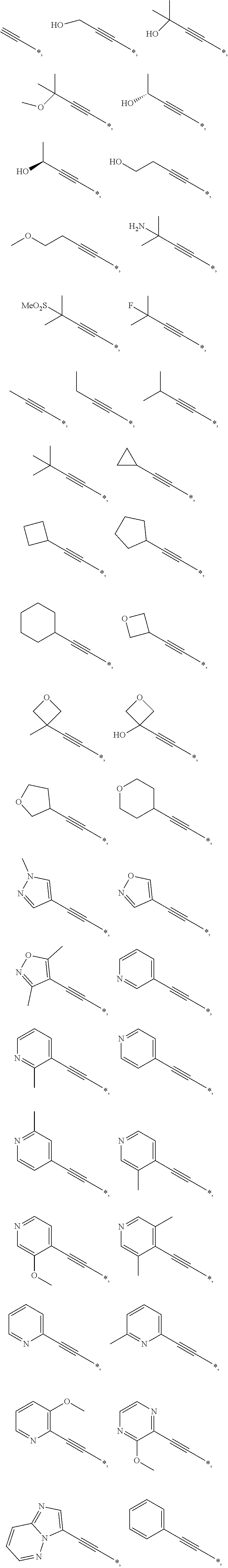

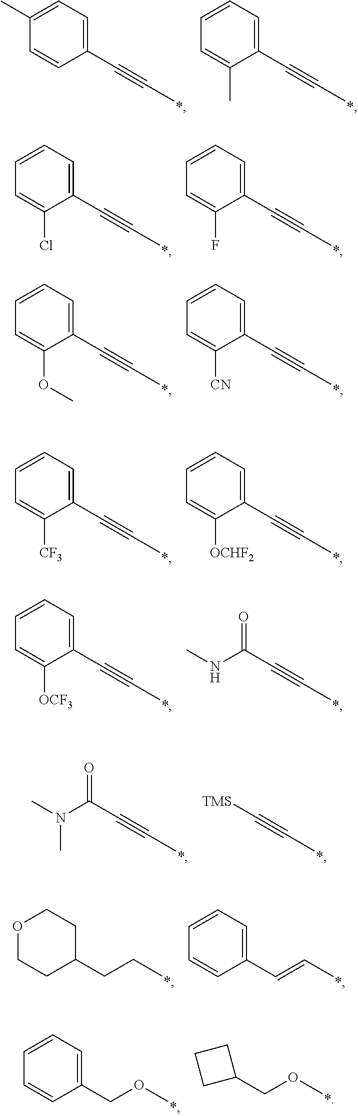

5. The compound of embodiment 4, wherein, each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(=NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.
6. The compound of anyone of embodiment 1-5, wherein, R³ is H, D, halogen, CN, C₁-C₄ alkyl, C₁-C₄haloalkyl.
7. The compound of anyone of embodiment 1-2, 6, wherein, R² and R³ together with the atoms to which they are attached form phenyl, 5-6 membered heteroaryl; wherein, the phenyl, 5-6 membered heteroaryl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}(O)NR^{c}R^{d}, NR^{c}C(O)OR^{a}; preferably, R² and R³ together with the atoms to which they are attached form phenyl, pyrrolyl, imidazolyl, pyrazolyl; each is optionally substituted by 1, 2, 3, 4, or 5 substituents independently selected from D, halo, CN, oxo, C₁-C₆ alkyl, C₁-C₆ haloalkyl, C₁-C₆ alkyl-OHA, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, N^{c}C(O)R^{b}, —NR^{c}(O)NR^{c}R^{d}, NR^{c}RC(O)OR^{a}.
8. The compound of anyone of embodiment 1-3, wherein, R² and R³ together with the atoms to which they are attached form phenyl, 1-methyl-1H-pyrazolyl.
9. The compound of anyone of embodiment 1-8, wherein, R⁴ is H, D, halogen, CN.
10. The compound of anyone of embodiment 1-9, wherein, R⁵ is selected from H, D, halogen, OH, CH₃, CF₃, CHF₂, CH₂F.
11. The compound of anyone of embodiment 1-10, wherein, R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂NR^{6A}R^{6B}, S(O)(═NR^{6A})R^{6B}.
12. The compound of anyone of embodiment 1-2, wherein, R⁶ is F, Cl, Br, I, SCH₃, S(O)CH₃, S(O)₂CH₃, S(O)₂CH₂CH₃, S(O)₂CH(CH₃)₂, S(O)₂CH₂CH(CH₃)₂, S(O)₂CF₃, S(O)₂CH₂CF₃, OS(O)₂CH₃, OS(O)₂CF₃, OTs,

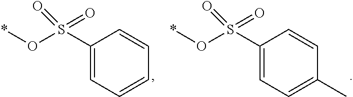

13. The compound of anyone of embodiment 1-12, wherein, Y¹ is N.
14. The compound of anyone of embodiment 1-12, wherein, Y¹ is CR⁷; and R⁷ is H, D; preferably, Y¹ is CH.
15. The compound of anyone of embodiment 1-14, wherein, Y² is N.
16. The compound of anyone of embodiment 1-14, wherein, Y² is CR⁸; and R⁸ is H, D, halo, OH, C₁-C₄ alkyl, C₁-C₄ haloalkyl.
17. The compound of anyone of embodiment 1-16, wherein, R⁹ is selected from CF₃, SF₅, CN, NC, NO₂.
18. The compound of anyone of embodiment 1-16, wherein, R⁸ and R⁹ together with the atoms to which they are attached form 5-membered heteroaryl optionally substituted by oxo; preferably, R⁸ and R⁹ together with the atoms to which they are attached form

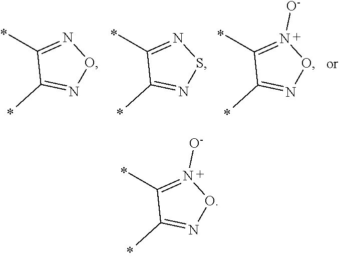

19. The compound of anyone of embodiment 1-18, wherein, Y³ is N.
20. The compound of anyone of embodiment 1-18, wherein, Y³ is CR¹⁰; and R¹⁰ is H, D; preferably, Y³ is CH.
21. The compound of anyone of embodiment 1-20, wherein, the compounds of Formula (I) are represented by compounds of Formula (II):

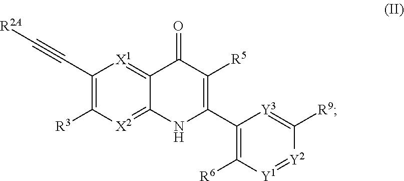

or a pharmaceutically acceptable salt, stereoisomer, solvate, N-oxide, tautomer, isotopic variant, prodrug or deuterated compound thereof;
wherein, X¹, X², Y¹, Y², Y³, R^{2A}, R⁵, R⁶, R³ and R⁹ are defined with respect to Formula (I).
22. The compound of embodiment 21, wherein, R^{2A} is selected from H, D, CN, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, C(O)R^{B}, C(O)NR^{C}R^{D}, C(O)OR^{A}, S(O)R^{B}, S(O)NR^{C}R^{D}, S(O)₂R^{B}, S(O)₂NR^{C}R^{D}, SiR^{G}R^{H}R^{I}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR_{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R, S(O)₂NR^{c}R^{d}, NR^{c}S(O)₂NR^{c}R^{d}, S(O)(=NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.
23. The compound of embodiment 22, wherein, R^{2A} is selected from H, D, CN, C₁-C₆ alkyl, C₆-C₁₀ aryl, C₃-C₁₀ cycloalkyl, 5-10 membered heteroaryl, 4-10 membered heterocycloalkyl; wherein each is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(═NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.
24. The compound of embodiment 22, wherein, R^{2A} is H, D, CN, CH₃, CH₂CH₃, CH(CH₃)₂,

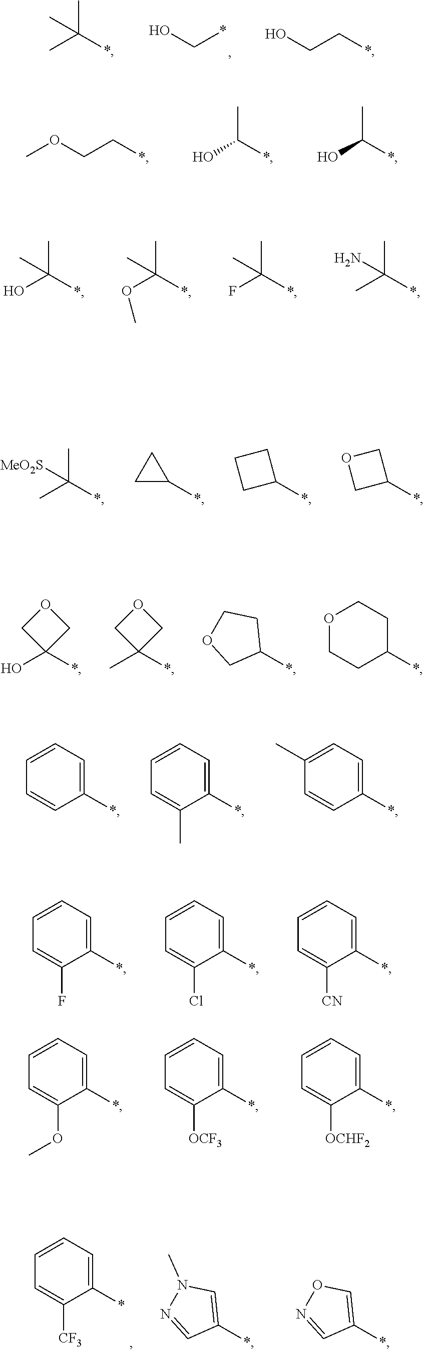

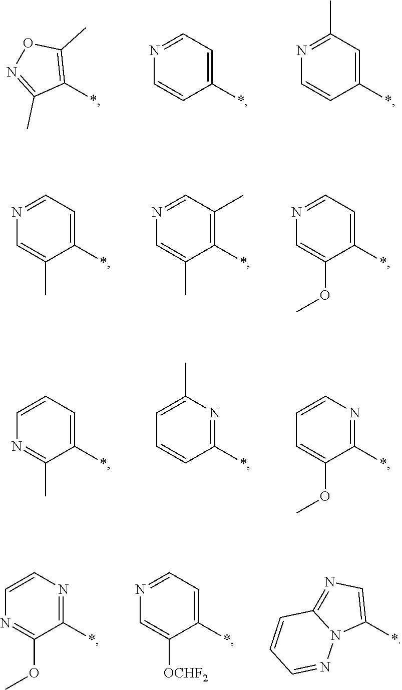

25. The compound of anyone of embodiment 1-23, wherein, the compound of Formula (I) is:

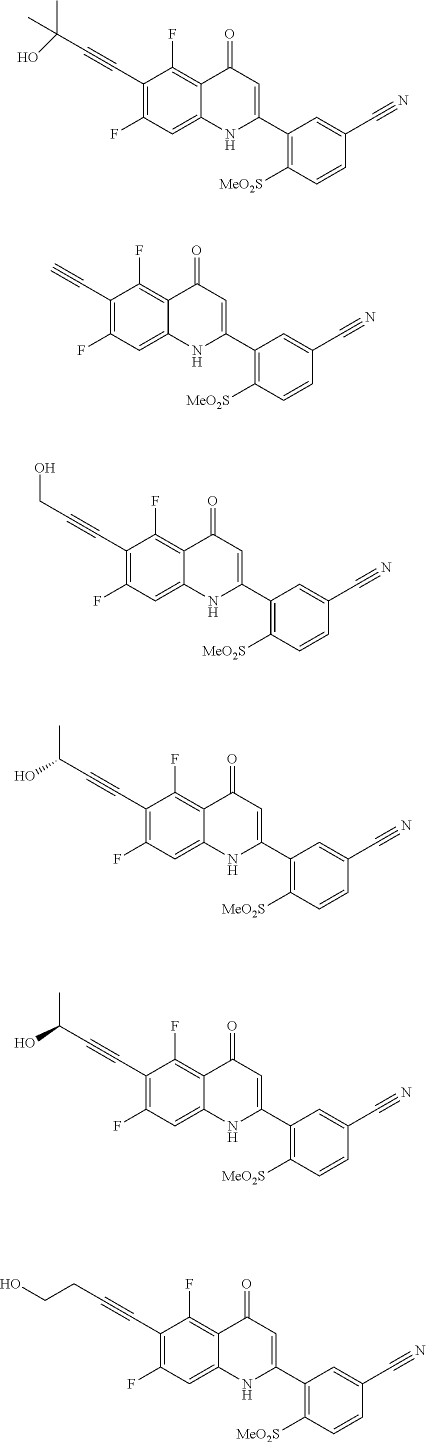 [SMILES: CC(C)(O)C#Cc1c(F)cc2[nH]c(-c3cc(C#N)ccc3S(C)(=O)=O)cc(=O)c2c1F ; C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CCO)c(F)cc2[nH]1 ; C[C@@H](O)C#Cc1c(F)cc2[nH]c(-c3cc(C#N)ccc3S(C)(=O)=O)cc(=O)c2c1F ; C[C@H](O)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CCCO)c(F)cc2[nH]1]

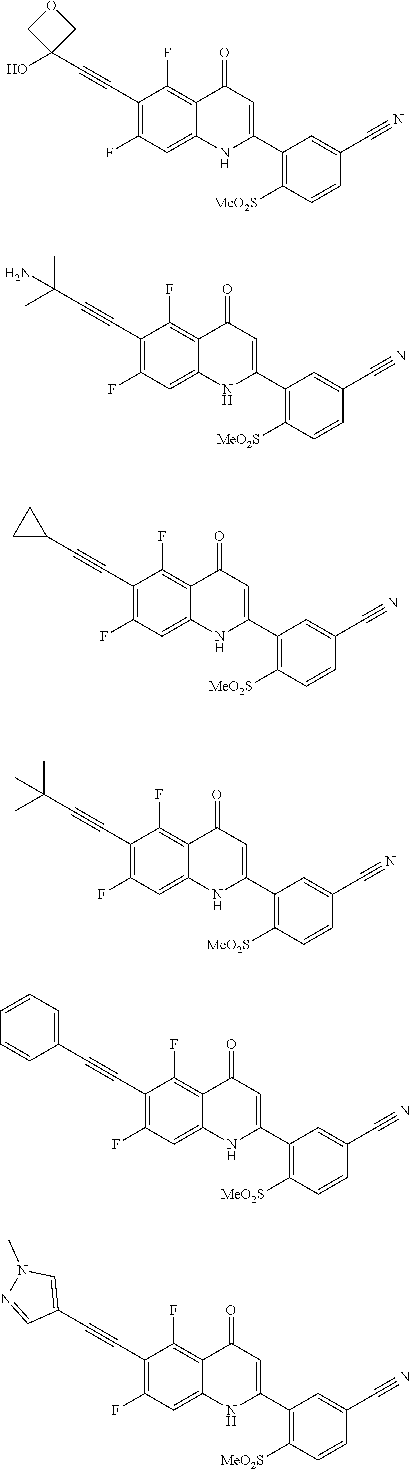 [SMILES: CS(=O)(=O)c1ccc(C#N)cc1-c1cc(=O)c2c(F)c(C#CC3(O)COC3)c(F)cc2[nH]1 ; CC(C)(N)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CC3CC3)c(F)cc2[nH]1 ; CC(C)(C)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3)c(F)cc2[nH]1 ; Cn1cc(C#Cc2c(F)cc3[nH]c(-c4cc(C#N)ccc4S(C)(=O)=O)cc(=O)c3c2F)cn1]

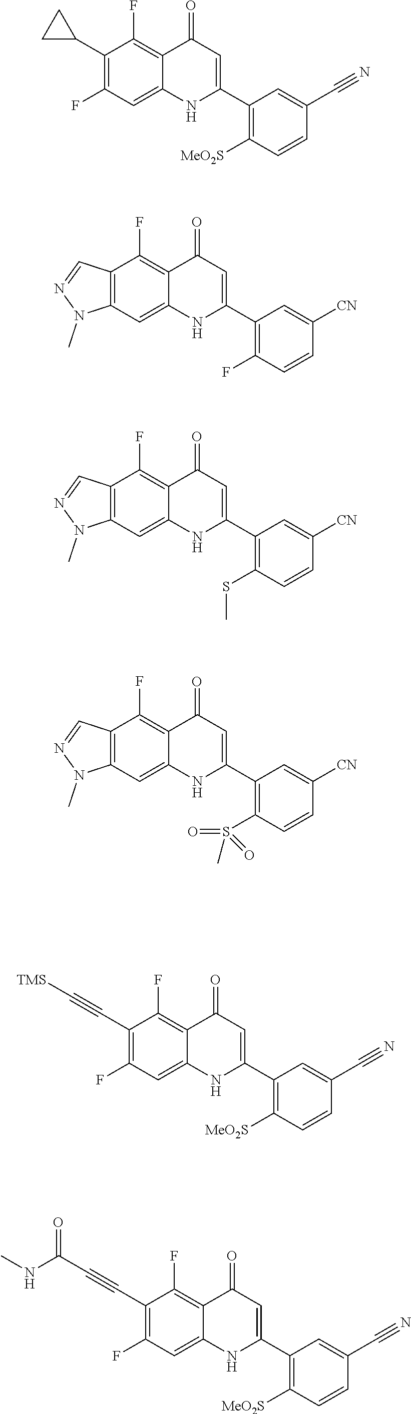 [SMILES: CS(=O)(=O)c1ccc(C#N)cc1-c1cc(=O)c2c(F)c(C3CC3)c(F)cc2[nH]1 ; Cn1ncc2c(F)c3c(=O)cc(-c4cc(C#N)ccc4F)[nH]c3cc21 ; CSc1ccc(C#N)cc1-c1cc(=O)c2c(F)c3cnn(C)c3cc2[nH]1 ; Cn1ncc2c(F)c3c(=O)cc(-c4cc(C#N)ccc4S(C)(=O)=O)[nH]c3cc21 ; C[Si](C)(C)C#Cc1c(F)cc2[nH]c(-c3cc(C#N)ccc3S(C)(=O)=O)cc(=O)c2c1F ; CNC(=O)C#Cc1c(F)cc2[nH]c(-c3cc(C#N)ccc3S(C)(=O)=O)cc(=O)c2c1F]

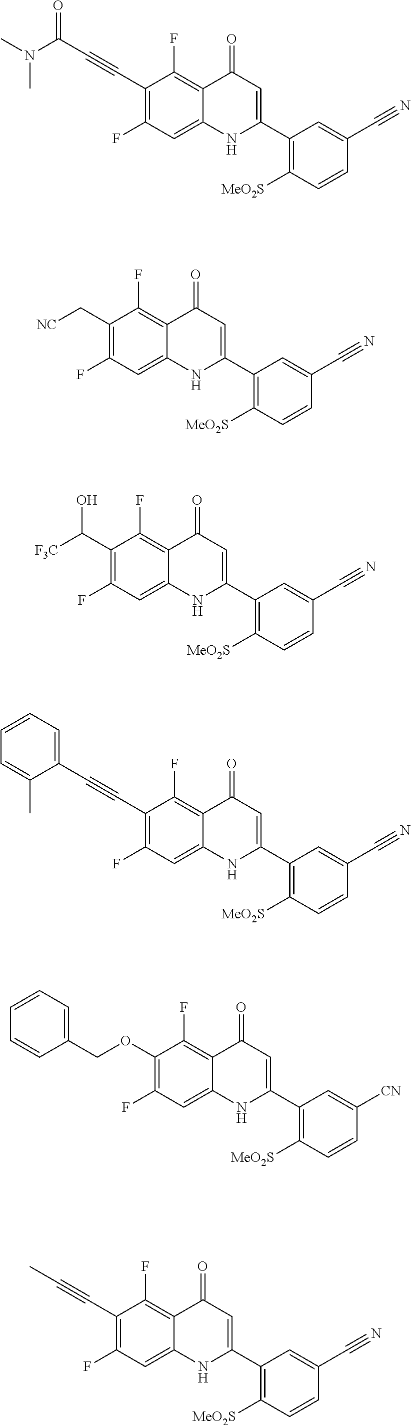 [SMILES: CN(C)C(=O)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(CC#N)c(F)cc2[nH]1 ; CS(=O)(=O)c1ccc(C#N)cc1-c1cc(=O)c2c(F)c(C(O)C(F)(F)F)c(F)cc2[nH]1 ; Cc1ccccc1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(OCc3ccccc3)c(F)cc2[nH]1 ; CC#Cc1c(F)cc2[nH]c(-c3cc(C#N)ccc3S(C)(=O)=O)cc(=O)c2c1F]

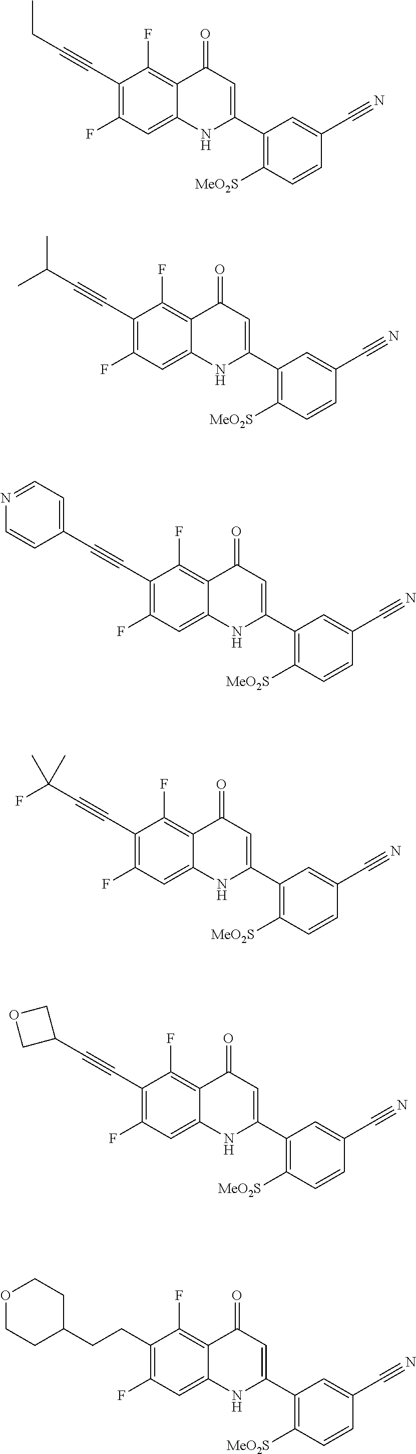 [SMILES: CCC#Cc1c(F)cc2[nH]c(-c3cc(C#N)ccc3S(C)(=O)=O)cc(=O)c2c1F ; CC(C)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccncc3)c(F)cc2[nH]1 ; CC(C)(F)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#CC3COC3)c(F)cc2[nH]1 ; CS(=O)(=O)c1ccc(C#N)cc1-c1cc(=O)c2c(F)c(CCC3CCOCC3)c(F)cc2[nH]1]

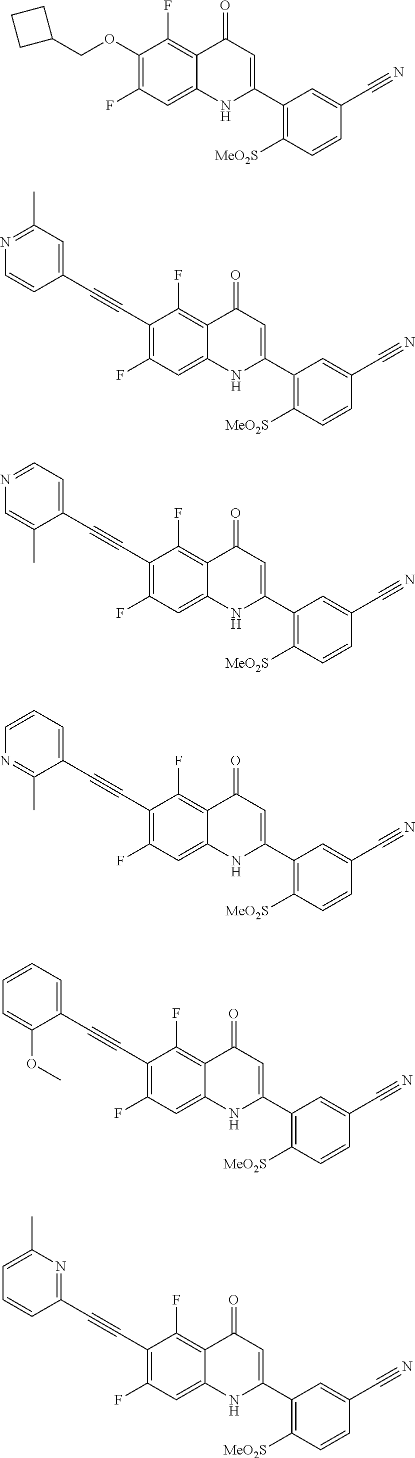 [SMILES: CS(=O)(=O)c1ccc(C#N)cc1-c1cc(=O)c2c(F)c(OCC3CCC3)c(F)cc2[nH]1 ; Cc1cc(C#Cc2c(F)cc3[nH]c(-c4cc(C#N)ccc4S(C)(=O)=O)cc(=O)c3c2F)ccn1 ; Cc1cnccc1C#Cc1c(F)cc2[nH]c(-c3cc(C#N)ccc3S(C)(=O)=O)cc(=O)c2c1F ; Cc1ncccc1C#Cc1c(F)cc2[nH]c(-c3cc(C#N)ccc3S(C)(=O)=O)cc(=O)c2c1F ; COc1ccccc1C#Cc1c(F)cc2[nH]c(-c3cc(C#N)ccc3S(C)(=O)=O)cc(=O)c2c1F ; Cc1cccc(C#Cc2c(F)cc3[nH]c(-c4cc(C#N)ccc4S(C)(=O)=O)cc(=O)c3c2F)n1]

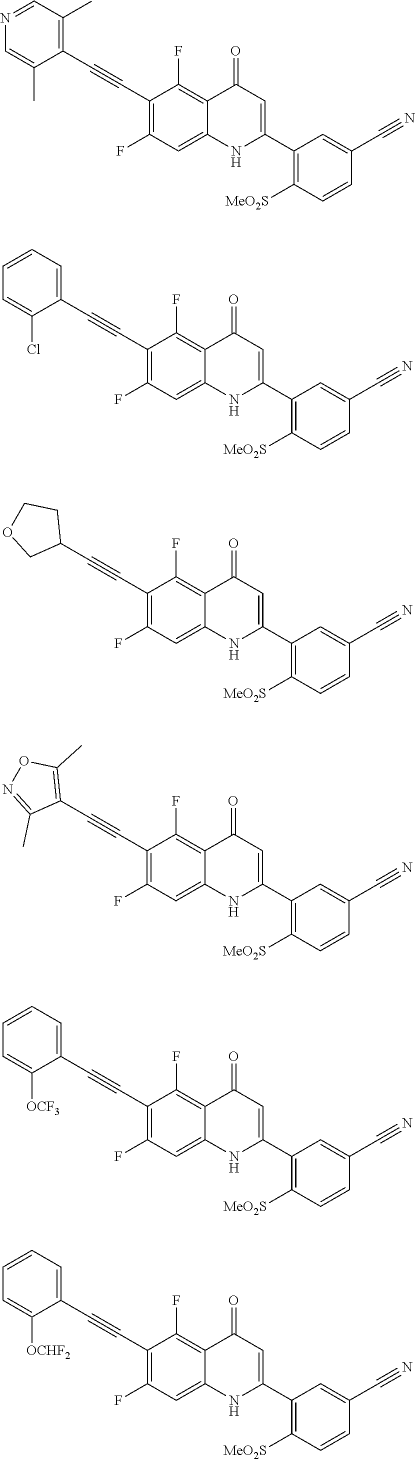 [SMILES: Cc1cncc(C)c1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3Cl)c(F)cc2[nH]1 ; CS(=O)(=O)c1ccc(C#N)cc1-c1cc(=O)c2c(F)c(C#CC3CCOC3)c(F)cc2[nH]1 ; Cc1noc(C)c1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3OC(F)(F)F)c(F)cc2[nH]1 ; CS(=O)(=O)c1ccc(C#N)cc1-c1cc(=O)c2c(F)c(C#Cc3ccccc3OC(F)F)c(F)cc2[nH]1]

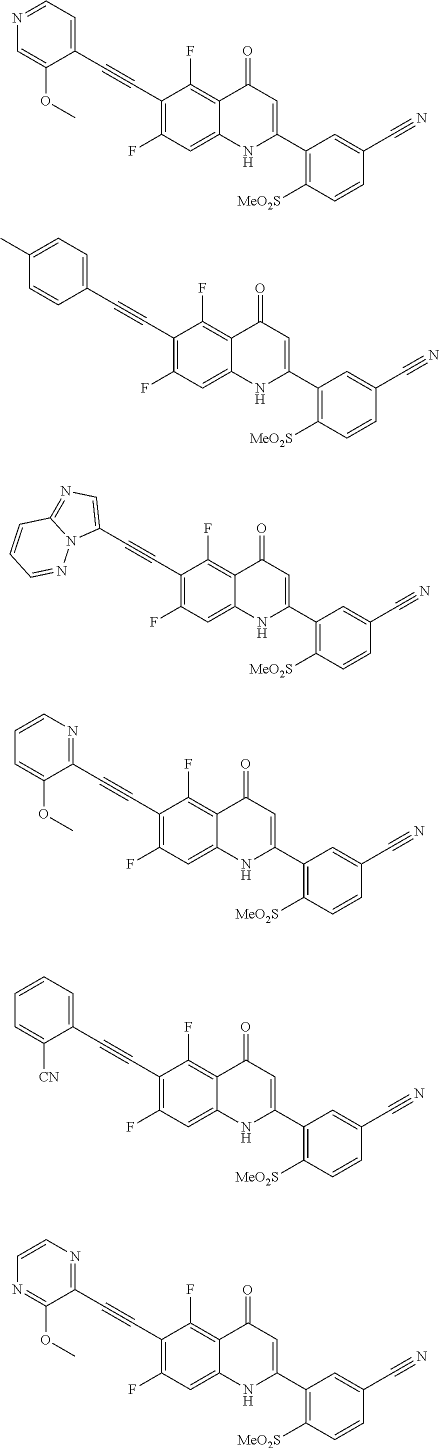 [SMILES: COc1cnccc1C#Cc1c(F)cc2[nH]c(-c3cc(C#N)ccc3S(C)(=O)=O)cc(=O)c2c1F ; Cc1ccc(C#Cc2c(F)cc3[nH]c(-c4cc(C#N)ccc4S(C)(=O)=O)cc(=O)c3c2F)cc1 ; CS(=O)(=O)c1ccc(C#N)cc1-c1cc(=O)c2c(F)c(C#Cc3cnc4cccnn34)c(F)cc2[nH]1 ; COc1cccnc1C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3C#N)c(F)cc2[nH]1 ; COc1nccnc1C#Cc1c(F)cc2[nH]c(-c3cc(C#N)ccc3S(C)(=O)=O)cc(=O)c2c1F]

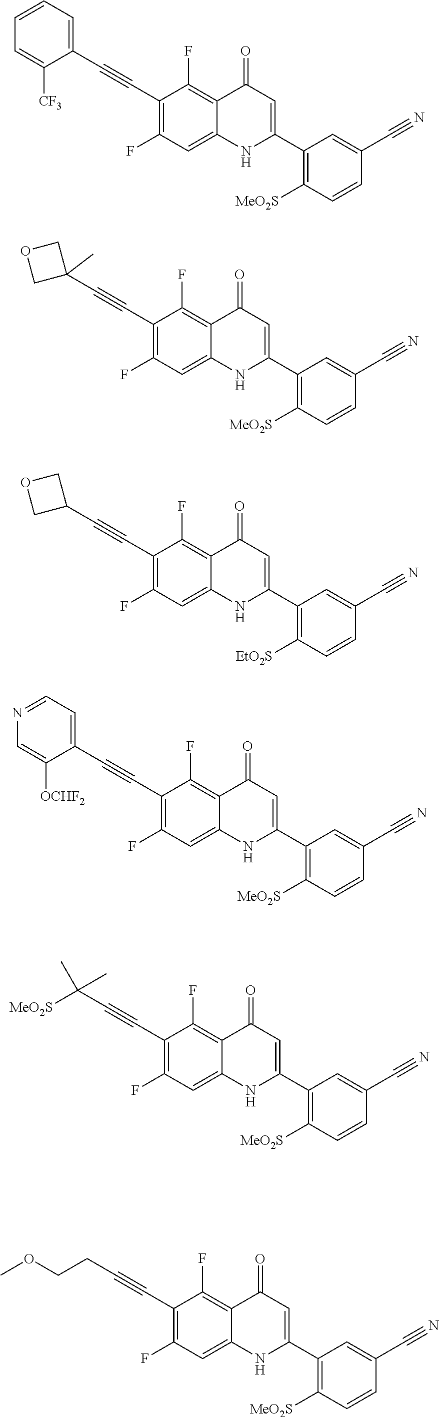

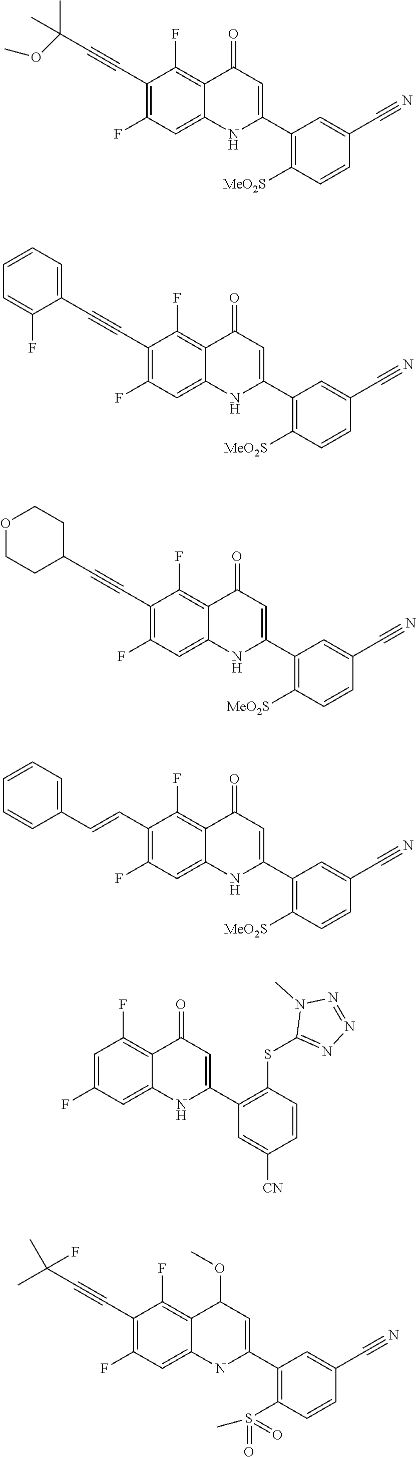 [SMILES: COC(C)(C)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccccc3F)c(F)cc2[nH]1 ; CS(=O)(=O)c1ccc(C#N)cc1-c1cc(=O)c2c(F)c(C#CC3CCOCC3)c(F)cc2[nH]1 ; CS(=O)(=O)c1ccc(C#N)cc1-c1cc(=O)c2c(F)c(/C=C/c3ccccc3)c(F)cc2[nH]1 ; Cn1nnnc1Sc1ccc(C#N)cc1-c1cc(=O)c2c(F)cc(F)cc2[nH]1 ; COC1C=C(c2cc(C#N)ccc2S(C)(=O)=O)Nc2cc(F)c(C#CC(C)(C)F)c(F)c21]

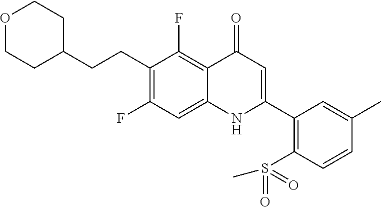 [SMILES: Cc1ccc(S(C)(=O)=O)c(-c2cc(=O)c3c(F)c(CCC4CCOCC4)c(F)cc3[nH]2)c1]

or a pharmaceutically acceptable salt thereof
26. A pharmaceutical composition comprising the compound of anyone of embodiment 1-24, a pharmaceutically acceptable salt, stereoisomer, solvate, N-oxide, tautomer, isotopic variant, prodrug, or deuterated compound thereof, and at least one pharmaceutically acceptable carrier or excipient.
27. Use of the compound of anyone of embodiment 1-24, or a pharmaceutically acceptable salt, stereoisomer, solvate, N-oxide, tautomer, isotopic variant, prodrug, or deuterated compound thereof for the preparation of a medicament.
28. A method of in vitro recruit co-repressor or block co-activator to PPARγ protein, comprising: contacting PPARγ protein with the compound of anyone of embodiment 1-24, a pharmaceutically acceptable salt or a pharmaceutically acceptable salt, stereoisomer, solvate, N-oxide, tautomer, isotopic variant, prodrug, or deuterated compound, or the pharmaceutical composition of embodiment 25.
29. A method of modulating PPARγ in cell, which is associated with activated function of PPARγ (gain-of-function mutation, amplification or overexpression) or RXRα gain-of-function mutation (such as S427F/Y), comprising: contacting the cell with the compound of anyone of embodiment 1-24, a pharmaceutically acceptable salt or a pharmaceutically acceptable salt, stereoisomer, solvate, N-oxide, tautomer, isotopic variant, prodrug, or deuterated compound, or the pharmaceutical composition of embodiment 25.
30. A method of treating a subject in need thereof comprising administering to the subject the compound of anyone of embodiment 1-24, a pharmaceutically acceptable salt or a pharmaceutically acceptable salt, stereoisomer, solvate, N-oxide, tautomer, isotopic variant, prodrug, or deuterated compound, or the pharmaceutical composition of embodiment 25.
31. The method of embodiment 29, wherein, the subject is suffering from, and is in need of a treatment for, a disease or disorder having the symptom of PPARγ amplification, overexpression and gain-of-function mutation or RXRα gain-of-function mutation.
32. The method of embodiment 30, wherein, the disease or disorder is a cancer.
33. The method of embodiment 31, wherein, the cancer is a cancer with activated function of PPARγ (gain-of-function mutation, amplification or overexpression) or RXRα gain-of-function mutation (such as S427F/Y).
34. The method of embodiment 31 or 32, wherein, the cancer is Bladder cancer.
35. The method of embodiment 30, wherein, the subject is human.

The following Examples are provided to illustrate some of the concepts described within this disclosure. While the Examples are considered to provide an embodiment, it should not be considered to limit the more general embodiments described herein.

Abbreviations

| | |
|---|---|
| aq. | Aqueous |
| AcOH | Acetic acid |
| BINAP | (2,2′-Bis(diphenylphosphino)-1,1′-binaphthyl) |
| Bippyphos | 5-(Di-tert-butylphosphino)-1′,3′,5′-triphenyl-1′H-[1,4′]bipyrazole |
| BrettPhos Pd G3 | [(2-Di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| | methanesulfonate |
| brine | Saturated solution of sodium chloride |
| BOP | (Benzotriazollyloxy)tris(dimethylamino)phosphonium |
| | hexafluophosphate |
| BSA | Bovine albumin |
| DAST | Diethylaminosulfur trifluoride |
| DCM | Dichloromethane |
| DIEA or DIPEA | N,N-diisopropylethylamine |
| DMAP | 4-Dimethylaminopyridine |
| DMF | N,N-dimethylformamide |
| DMSO | Dimethylsulfoxide |
| DBU | 1,8-Diazabicyclo[5.4.0]undec-7-ene |
| dppf | 1,1′-Bis(diphenylphosphino)ferrocene |
| EDTA | Ethylene diamine tetra-acetic acid |
| EtOAc or EA | Ethyl acetate |
| EtOH | Ethanol |
| Et₂O | Ethyl ether |
| EDCI | 1-Ethyl-3-(3-dimethylaminopropyl)carbodiimide hydrochloride |
| EtMgBr | Ethylmagnesium Bromide |
| FBS | Fetal Bovine Serum |
| HATU | 2-(7-Azabenzotriazol-1-yl)-N,N,N′,N′-tetramethyluronium |
| | hexafluorophosphate |
| HBTU | 1-[Bis(dimethylamino)methylene]-1H-benzotriazolium 3-Oxide |
| | Hexafluorophosphate |
| [HP(tBu)₃]BF₄ | Tri-tert-butylphosphonium tetrafluoroborate |
| ¹H NMR | Hydrogen-1 nuclear magnetic resonance spectroscopy |
| KHMDS | Potassium bis(trimethylsilyl)amide |
| KAuCl₄ | Goldpotassium chloride |
| LiHMDS | Lithium bis(trimethylsilyl)amide |
| LDA | Lithium diisopropylamide |
| LiOtBu | Lithium tert-butoxide |
| LCMS | Liquid chromatography-mass spectrometry |
| MeCN/ACN | Acetonitrile |
| MeOH | Methanol |
| MTBE | Methyl tert-butyl ether |
| n-BuLi | n-Butyllithium |
| NaHMDS | Sodium bis(trimethylsilyl)amide |
| NaOMe | Sodium methoxide |
| NaOEt | Sodium ethoxide |
| NaBH(OAc)₃ | Sodium Triacetoxyborohydride |
| NaBH₃CN | Sodium cyanoborohydride |
| NBS | N-Bromosuccinimide |
| NMI | N-Methyl-Imidazole |
| Pd(dppf)Cl₂ | [1,1′-Bis(diphenylphosphino)ferrocene]dichloropalladium(II) |
| Pd(OAc)₂ | Palladium (II) Acetate |
| Pd(PPh₃)₄ | Tetrakis(triphenylphosphine)palladium(0) |
| Pd(PPh₃)₂Cl₂ | Bis(triphenylphosphine)palladium(II) chloride |
| Pd₂(dba)₃ | Bis(dibenzylideneacetone)palladium(0) |
| PE | Petroleum ether |
| PPA | polyphosphoric acid |
| Q-phos | 1,2,3,4,5-Pentaphenyl-1′-(di-tert-butylphosphino)ferrocene |
| PPh₃AuNTf₂ | [bis(trifluoromethanesulfonyl)imidate](triphenylphosphine)gold(I) |
| P(OEt)₃ | Triethylphosphine |
| PyBOP | Benzotriazole-1-yl-oxytripyrrolidinophosphonium |
| | hexafluorophosphate |
| r.t. | Room temperature |
| RockPhos | 2-(Di-t-butylphosphino)-3-methoxy-6-methyl-2′,4′,6′-tri-i-propyl-1,1′- |
| | biphenyl |
| RuPhos Pd G3 | (2-Dicyclohexylphosphino-2′,6′-diisopropoxy-1,1′-biphenyl)[2-(2′- |
| | amino-1,1′-biphenyl)]palladium(II) methanesulfonate |
| TEMPO | 2,2,6,6-Tetramethylpiperidoxyl |
| t-BuOK | Potassium tert-butoxide |
| t-BuONa | Sodium tert-butoxide |
| TEA or Et₃N | Triethylamine |
| TFA | Trifluoroacetic acid |
| THF | Tetrahydrofuran |
| TBAF | Tetra-n-Butylammonium Fluoride |
| TCFH | Chloro-N,N,N′,N′-tetramethylformamidinium hexafluorophosphate |
| T₃P | Propylphosphonic anhydride |

1M or 1N = 1 mol/L, 2M or 2N = 2 mol/L etc.

### General Synthetic Schemes

A series of 4-quinolone derivatives of Formula (I) can be prepared as the methods outlined in Scheme 1. Camps cyclization of the amide derivatives 1-1 under basic conditions such as NaOH, KOH, LiOtBu or KOtBu in a suitable solvent such as 1,4-dioxane, 2-methyltetrahydrofuran, Et₂O or THF can provide the 4-quinolone derivatives of Formula (I). In a similar manner, intramolecular oxidative Mannich reactions of the 2-ketoaniline derivatives 1-2 with TEMPO in the presence of a base such as NaOtBu or KOtBu in DMSO or DMF can also yield the 4-quinolone derivatives of Formula (I).

Alternatively, a gold-catalyzed intramolecular cyclization of aryl acetylenic ketone derivatives 1-3 in the presence of a catalyst such as PPh₃AuNTf₂, KAuCl₄ or IMes-AuNTf₂ in MeCN can afford the 4-quinolone derivatives of Formula (I).

Further alternatively, the 4-quinolone derivatives of Formula (I) can be prepared by Conrad-Limpach cyclization of anilines 1-4 with benzoylacetates 1-5 under acidic conditions such as H₃PO₄, PPA or H₂SO₄ in high boiling point solvent (e.g., xylene, Ph₂O, Dowtherm A).

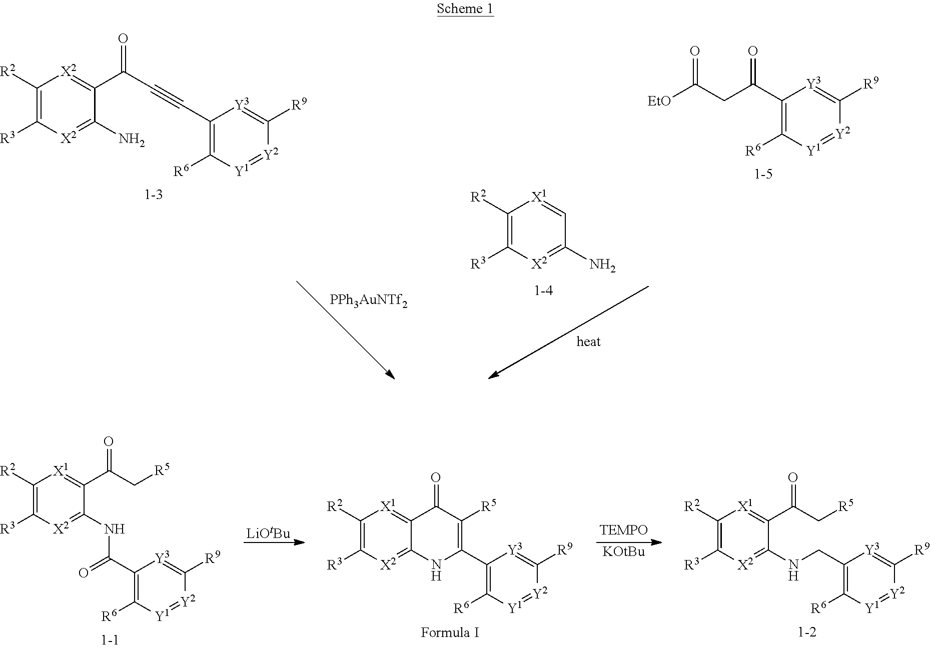

A series of amide derivatives of formula 2-1 can be prepared as the methods outlined in Scheme 2. Amide coupling of carboxylic acids 2-2 with 2-ketoanilines 2-4 under conditions (e.g., in the presence of an activating reagent such as BOP, PyBOP, HATU, HBTU, EDCI, TCFH or T₃P and a base, such as Hunig's base, TEA, pyridine, NMI or DMAP) can afford the amide derivatives of formula 2-1. Alternatively, treatment of the carboxylic acids 2-2 with the chlorine reagent such as oxalyl dichloride, thionyl chloride, or POCl₃ can produce the corresponding acid chlorides of formula 2-3 which is subsequently coupled with 2-ketoanilines 2-4 under a base such as TEA, DIEA or DMAP can also provide the amide derivatives of formula 2-1.

Alternatively, Ullmann coupling of compounds 2-5 where W is a halogen (Br, Cl, I) or pseudohalogen (e.g., OTf or OMs) with appropriate benzamide derivatives 2-6 under standard Ullmann conditions (e.g., CuI, K₃PO₄ and ligand such as proline derivatives, BINAP et. al) or under standard Buchwald-Hartwig coupling conditions (e.g., Pd₂(dba)₃, Bippyphos, RockPhos, RuPhos Pd G3), BrettPhos Pd G3, Q-phos et. al. and a base, such as t-BuOK, K₃PO₄, Cs₂CO₃, or K₂CO₃) can also provide the amide derivatives of formula 2-1.

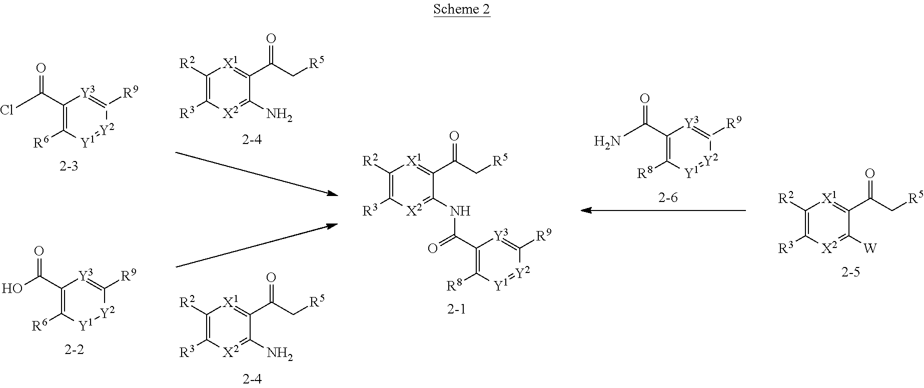

A series of N-substitute 2-ketoaniline derivatives of formula 3-1 can be prepared as the methods outlined in Scheme 3. Reductive amination of aldehydes 3-2 with 2-ketoanilines 3-2 under conditions (e.g., in the presence of a catalytic reagent such as HOAc, TFA, or H₂SO₄ and a reductant, such as NaBH(OAc)₃ or NaBH₃CN) can afford the 2-ketoaniline derivatives of formula 3-1.

Alternatively, coupling of compounds 3-4 where W is a halogen (Br, I) or pseudohalogen (e.g., OTf) with appropriate benzylamine derivatives 3-5 in the presence of a base (e.g., TEA, DBU, KOtBu et. al) or under Buchwald-Hartwig coupling conditions (e.g., Pd₂(dba)₃, Bippyphos, RuPhos Pd G3, BrettPhos Pd (33, RockPhos, Q-phos and a base, such as t-BuOK, K₃PO₄, Cs₂CO₃, or K₂CO₃) can also provide the N-substitute 2-ketoaniline derivatives of formula 3-1.

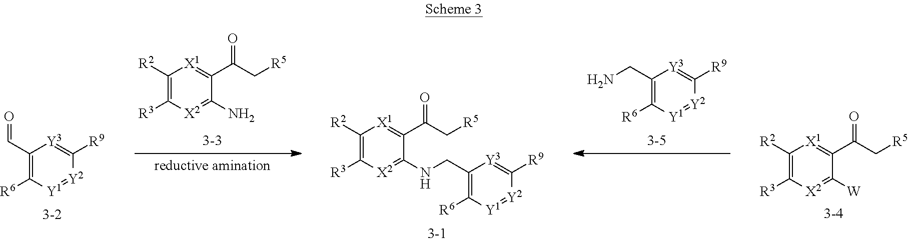

A series of aryl acetylenic ketone derivatives of formula 4-1 can be prepared as the methods outlined in Scheme 4. Sonogashira reaction of 2-haloanilines 4-2 where W is a halogen (Br, I) or pseudohalogen (e.g., OT) with aryl acetylenes 4-4 under conditions (e.g., in the presence of a palladium-catalyst such as Pd(dppf)Cl₂, Pd(PPh₃)₂Cl₂, Pd₂dba)₃ or Pd(OAc)₂ with a ligand, such as dppf or [HP(tBu)₃]BF₄ and a base such as TEA or NHEt₂) can afford the N-protected acetylenic ketone derivatives of formula 4-7 which can be transformed into compounds 4-1 under acidic conditions such as TFA or HCl.

Alternatively, N-protected acetylenic ketone derivatives of formula 4-7 can be prepared by coupling of Weinreb amides 4-6 with aryl acetylenes 4-4 under an appropriate base (e.g., LiHMDS, NaHMDS, KHMDS, LDA, EtMgBr or n-BuLi et. al) for deprotonation of acetylenic hydrogen. Similarly, coupling of aldehydes 4-3 with aryl acetylenes 4-4 under an appropriate base (e.g., LiHMDS, NaHMDS, KHMDS, LDA, EtMgBr or n-BuLi et. al) can provide the propargyl alcohol derivatives of formula 4-5 which is subsequently oxidized under an appropriate oxidant such as MnO₂, PCC, PDC, DMP or IBX to afford the N-protected acetylenic ketone derivatives of formula 4-7.

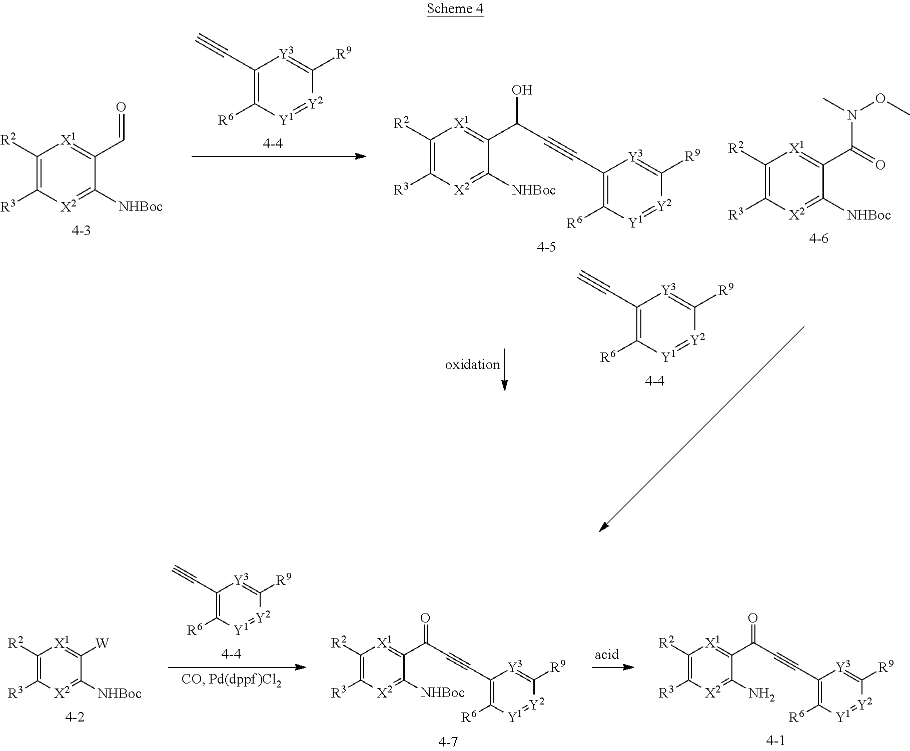

A series of 4-quinolone derivatives of formula 5-1 can be prepared as the methods outlined in Scheme 5. Camps cyclization of the amide derivatives 5-2 in the presence of a base such as NaOH, KOH, LiOtBu or KOtBu in a suitable solvent such as 1,4-dioxane, 2-methyltetrahydrofuran, Et₂O or THF can provides the 4-quinolone derivatives 5-3. Sonogashira coupling of the 4-quinolone derivatives 5-2 with alkyne derivatives 5-4 under standard conditions (e.g., in the presence of a co-catalytic reagent such as Pd(PPh₃)₂Cl₂/CuI, and a base, such as TEA or DIEA) can afford the 4-quinolone derivatives 5-1.

Alternatively, Sonogashira coupling of the amide derivatives 5-2 with alkyne derivatives 5-4 under standard conditions (e.g., in the presence of a co-catalytic reagent such as Pd(PPh₃)₂Cl₂/CuI, and a base, such as TEA or DIEA) can provide the compound 5-5 which is subsequently cyclized in the presence of a base such as NaOH, KOH, LiOtBu or KOtBu in a suitable solvent such as 1,4-dioxane, 2-methyltetrahydrofuran, Et₂O or THE to afford the 4-quinolone derivatives 5-1.

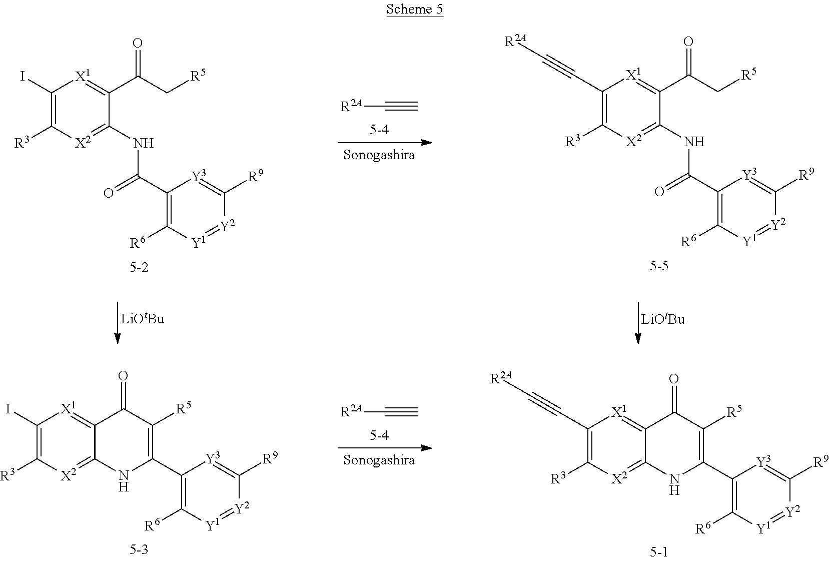

A series of 4-quinolone derivatives of formula 6-2 where R⁶ is OSO₂R^{6A}, SR^{6A} or SO₂R^{6A} can be prepared from 4-quinolone derivatives of 6-1 where R⁶ is Cl, Br, or I with HOSO₂R^{6A}, NaSR^{6A} or NaSO₂R^{6A} under a base such as NaH, KOtBu, DIEA or KOH as the methods outlined in Scheme 6.

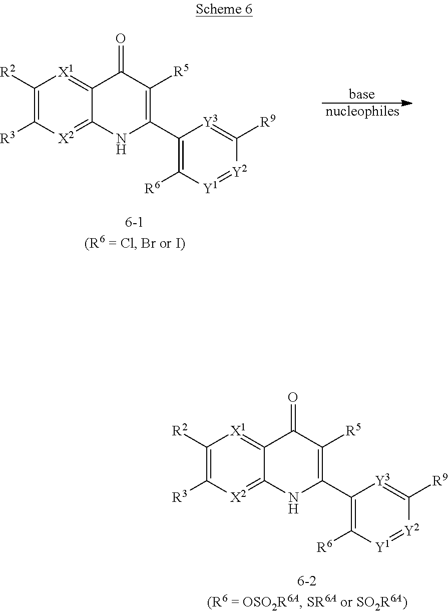

A series of 2-ketoanilines derivatives of formula 7-1 can be prepared as the methods outlined in Scheme 7. Weinreb amides 7-3 can be easily prepared from its acid precursors 7-2 with N,O-dimethylhydroxlamine under standard amide coupling conditions (e.g., in the presence of an amide coupling reagent such as BOP, PyBOP, HATU, HBTU or EDCI, and a base such as TEA, Hunig's base or pyridine). Treatment of Weinreb amides 7-3 with Grignard reagents 7-4 at low temperature in THF can provide the corresponding 2-ketoanilines derivatives 7-5. Deprotection of the compounds 7-5 under acidic conditions such as TFA or HCl can afford the 2-ketoanilines derivatives 7-1.

Alternatively, 2-ketoanilines derivatives 7-1 can be prepared from anilines 7-6 by Friedel-Crafts acylation with acyl chloride 7-7 under an appropriate acid (e.g., PPA, or AlCl₃ et. al).

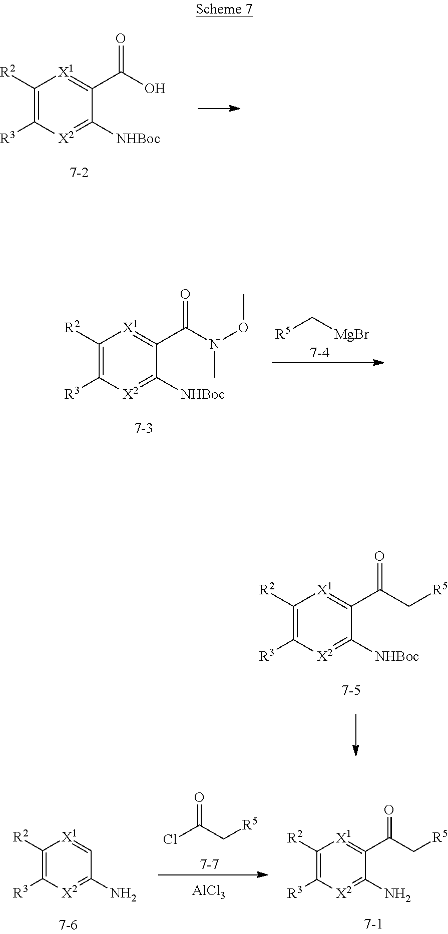

A series of benzoylacetate derivatives of formula 8-1 can be prepared as the methods outlined in Scheme 8. Claisen condensation of esters 8-2 with EtOAc under a base (e.g., NaOEt. NaOMe or KOtBu et. al) can produce the benzoylacetate derivatives 8-1. Alternatively, condensation of aryl methyl ketones 8-3 and diethyl carbonate under a base (e.g., NaH, LDA, NaOEt, NaOMe or KOtBu et. al) can also afford the benzoylacetate derivatives 8-1.

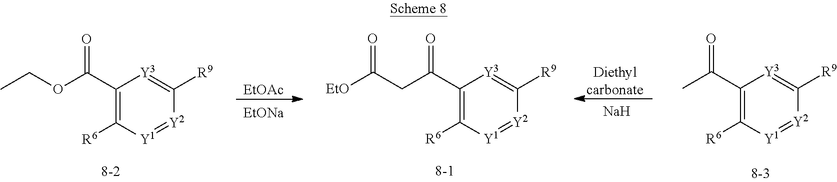

A series of aryl acetylene derivatives of formula 9-1 can be prepared as the methods outlined in Scheme 9. Sonogashira coupling of compounds 9-2 with trimethylsilylacetylene under standard conditions (e.g., in the presence of a co-catalytic reagent such as Pd(PPh₃)₂Cl₂/CuI, and a base, such as TEA or DIEA) can provide the compounds 9-3 which is subsequently deprotected under a base (e.g., K₂CO₃, KOH, NaOH or TBAF) or Olah reagent to afford the aryl acetylene derivatives 9-1. Alternatively, aryl acetylene derivatives 9-1 can be prepared by treatment of aldehydes 9-4 with Ohira-Bestmann reagent under an appropriate base (e.g., K₂CO₃, or KOtBu et. al).

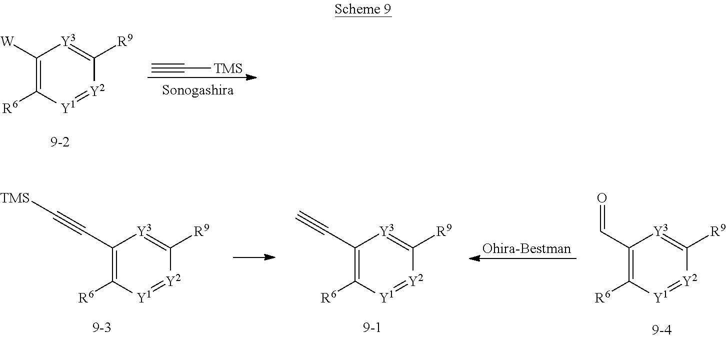

A series of benzoxadiazol derivatives of formula 10-1 can be prepared as the methods outlined in Scheme 10. Treatment of 2-nitroaniline derivatives 10-2 with KOH and NaClO can provide the compounds 10-3 which is subsequently reduced tinder a reducing agent P(OEt)₃ to afford the benzoxadiazol derivatives 10-1.

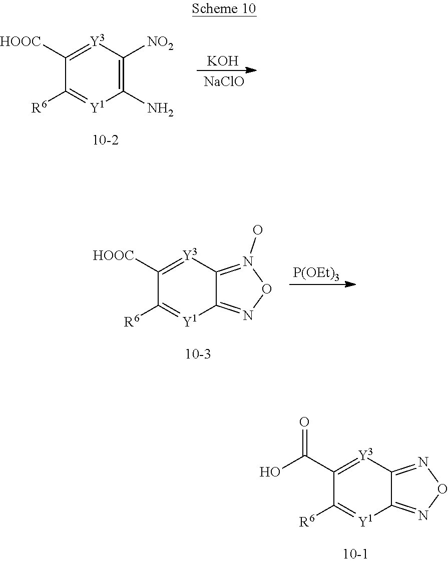

A series of benzothiadiazole derivatives of formula 11-1 can be prepared as the methods outlined in Scheme 10. Treatment of o-phenylenediamine derivatives 11-2 with SOCl₂ can provide the compounds 11-3 which is subsequently hydrolyzed under a base (e.g., LiOH, NaOH or KOH et. al) to afford the benzothiadiazole derivatives 11-1

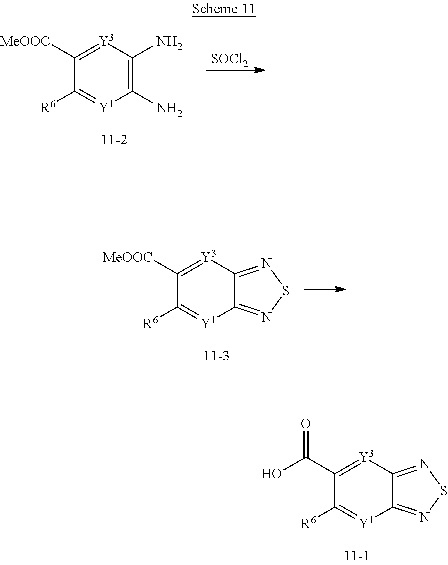

### EXAMPLES

### Example 1: 3-(5,7-Difluoro-6-(3-hydroxy-3-methylbut-1-yn-1-yl)-4-oxo-1,4-dihydroquinolin-2-yl)-4-(methylsulfonyl)benzonitrile

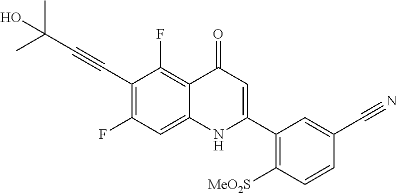 [SMILES: CC(C)(O)C#Cc1c(F)cc2[nH]c(-c3cc(C#N)ccc3S(C)(=O)=O)cc(=O)c2c1F]

### Step 1: 1-(2-amino-4,6-difluorophenyl)ethan-1-one

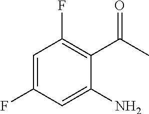

A mixture of 1-(2,4,6-trifluorophenyl)ethan-1-one (5 g, 28.7 mmol) and ammonium hydroxide (14 g, 230 mmol, 28% aq.) in 2-methyltetrahydrofuran (13 mL) was heated to 105° C. overnight in a sealed tube. The reaction mixture was cooled to 25° C. The aqueous layer was adjusted to pH~8-9 with aq. HCl (2.0 M) and extracted with EtOAc (50 mL×2). The combined organic layers were washed with brine, dried over Na₂SO₄, filtered and concentrated under reduced pressure. The residue was purified by flash chromatography on a silica gel column eluting with EtOAc/PE (5-12%) to afford the title compound (2.5 g) as a white solid. LCMS calc. for C₈H₈F₂NO [M+H]⁺: m/z 172.1; Found: 1721.

### Step 2: 1-(6-amino-2,4-difluoro-3-iodophenyl)ethan-1-one

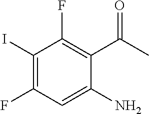

To a solution of 1-(2-amino-4,6-difluorophenyl)ethan-1-one (1.0 g, 5.8 mmol) in DCM (10 mL) was added N-iodosuccinimide (1.4 g, 6.4 mmol). The mixture was stirred at 20° C. overnight. The reaction mixture was diluted with water (10 mL) and extracted with DCM (20 mL×2). The combined organic layers were washed with brine, dried over Na₂SO₄, filtered and concentrated under reduced pressure. The residue was purified by flash chromatography on a silica gel column eluting with EtOAc/PE (10-30%) to afford the title compound (450 mg) as a white solid. LCMS calc. for C₈H₇F₂INO [M+H]⁺: m/z=298.0; Found: 298.0.

### Step 3: 5-cyano-2-(methylsulfonyl)benzoyl chloride

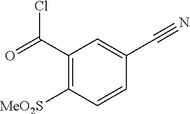 [SMILES: CS(=O)(=O)c1ccc(C#N)cc1C(=O)Cl]

A mixture of 5-cyano-2-(methylsulfonyl)benzoic acid (0.5 g, 2.2 mmol) in SOCl₂ (5 mL) was stirred at 80° C. for 2 h. and then concentrated under reduced pressure to afford the title compound (0.5 g) as a colorless oil.

### Step 4: N-(2-acetyl-3,5-difluoro-4-iodo phenyl)-5-cyano-2-(mehylsulfonyl)benzamide

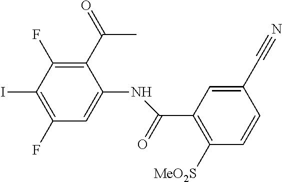 [SMILES: CC(=O)c1c(NC(=O)c2cc(C#N)ccc2S(C)(=O)=O)cc(F)c(I)c1F]

To a mixture of 1-(6-amino-2,4-difluoro-3-iodophenyl)ethan-1-one (0.6 g, 2.2 mmol) and TEA (0.4 g, 4.0 mmol) in DCM (10 mL) was added 5-cyano-2-(methylsulfonyl)benzoyl chloride (0.55 g, 2.2 mmol). The reaction mixture was stirred at r.t. overnight. The resulting mixture was concentrated under reduced pressure. The residue was purified by flash chromatography on a C18 column eluting with ACN/water (30-48%) to afford the title compound (0.58 g) as a white solid. LCMS calc. for C₁₇H₁₂F₂IN₂O₄S [M+H]⁺: m/z=505.0; Found: 504.9.

### Step 5: 3-(5,7-difluoro-6-iodo-4-oxo-1,4-dihydroquinolin-2-yl)-4-(methylsulfonyl)benzonitrile

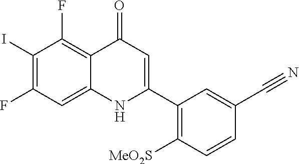 [SMILES: CS(=O)(=O)c1ccc(C#N)cc1-c1cc(=O)c2c(F)c(I)c(F)cc2[nH]1]

A mixture of N-(2-acetyl-3,5-difluoro-4-iodophenyl)-5-cyano-2-(methylsulfonyl)benzamide (35 mg, 0.07 mmol) and LiOtBu (6.7 mg, 0.08 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with ACN/H₂O (45-63% with 0.5 TFA) to afford the title compound (7 mg) as an off-white solid. LCMS calc. for C₁₇H₁₀F₂IN₂O₃S [M+H]⁺: m/z=486.9; Found: 486.9.

### Step 6: 3-(5,7-difluoro-6-(3-hydroxy-3-methylbut-1-yn-1-yl)-4-oxo-1,4-dihydroquinolin-2-yl)-4-(methylsulfonyl)benzonitrile

A mixture of CuI (0.4 mg 0.002 mmol), PdCl₂ (PPh₃)₂ (1.4 mg, 0.002 mmol), 3-(5,7-difluoro-6-iodo-4-oxo-1,4-dihydroquinolin-2-yl)-4-(methylsulfonyl)benzonitrile (10 mg, 0.02 mmol), and 2-methylbut-3-yn-2-ol (2.6 mg, 0.03 mmol) in DMF (0.2 ml) and TEA (0.1 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (15-25%) to afford the title compound (7 mg) as an off-white solid. LCMS calc. for C₂₂H₁₇F₂N₂O₄S [M+H]⁺: m/z=443.1; Found: 443.0.

### Example 2: 3-(6-Ethynyl-5,7-difluoro-4-oxo-1,4-dihydroquinolin-2-yl)-4-(methylsulfonyl)benzonitrile

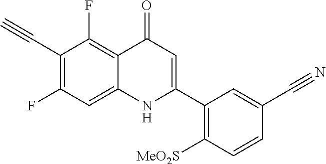 [SMILES: C#Cc1c(F)cc2[nH]c(-c3cc(C#N)ccc3S(C)(=O)=O)cc(=O)c2c1F]

### Step 1: N-(2-acetyl-3,5-difluoro-4-((trimethylsilyl)ethynylphenyl)-5-cyano-2-(methylsulfonyl)benzamide

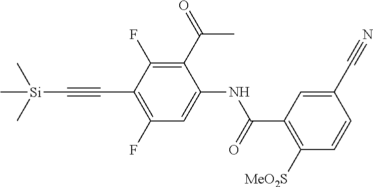 [SMILES: CC(=O)c1c(NC(=O)c2cc(C#N)ccc2S(C)(=O)=O)cc(F)c(C#C[Si](C)(C)C)c1F]

To a solution of trimethylsilylacetylene (15 mg, 0.15 mmol), N-(2-acetyl-3,5-difluoro-4-iodophenyl)-5-cyano-2-(methylsulfonyl)benzamide (50 tug, 0.1 mmol, Example 1 Step 4), CuI (2 mg, 0.01 mmol) and PdCl₂(PPh₃)₂ (7 mg, 0.01 mmol) in TEA (0.5 mL) and DMF (0.5 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (20-50%) to afford the title compound (25 mg) as an off-white solid. LCMS calc. for C₂₂H₂₁F₂N₂O₄SSi [M+H]⁺: m/z=475.1; Found: 475.1.

### Step 2: 3-(6-ethynyl-5,7-difluoro-4-oxo-1,4-dihydroquinolin-2-yl)-4-(methylsulfinyl)benzonitrile

A mixture of N-(2-acetyl-3,5-difluoro-4-((trimethylsilyl)ethynyl)phenyl)-5-cyano-2-(methylsulfonyl)benzamide (25 mg, 0.05 mmol) and LiOtBu (5 mg, 0.06 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with ACN/H₂O (25-46%) to afford the title compound (2.1 mg) as an off-white solid. LCMS calc. for C₁₉H₁₁F₂N₂O₃S [M+H]⁺: m/z=385.0; Found: 385.0.

### Example 3: 3-(5,7-Difluoro-6-(3-hydroxyprop-1-yn-1-yl)-4-oxo-1,4-dihydroquinolin-2-yl)-4-(methylsulfonyl)benzonitrile

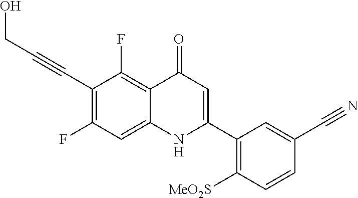 [SMILES: CS(=O)(=O)c1ccc(C#N)cc1-c1cc(=O)c2c(F)c(C#CCO)c(F)cc2[nH]1]

### Step 1: N-(2-acetyl-3,5-difluoro-4-(3-hydroxyprop-1-yn-1-yl)phenyl)-5-cyano-2-(methylsulfonyl)benzamide

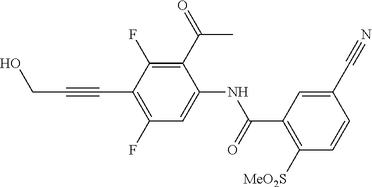 [SMILES: CC(=O)c1c(NC(=O)c2cc(C#N)ccc2S(C)(=O)=O)cc(F)c(C#CCO)c1F]

To a solution of trimethyl(prop-2-yn-1-yloxy)silane (19 mg, 0.15 mmol), N-(2-acetyl-3,5-difluoro-4-iodophenyl)-5-cyano-2-(methylsulfonyl)benzamide (50 mg, 0.1 mmol, Example 1 Step 4), CuI (2 mg, 0.01 mmol) and PdCl₂(PPh₃)₂ (7 mg, 0.01 mmol) in TEA (0.5 mL) and DMF (0.5 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (5-14%) to afford the title compound (15 mg) as an off-white solid. LCMS calc. for C₂₀H₁₅F₂N₂O₅S [M+H]⁺: m/z=433.1; Found: 433.0.

### Step 2: 3-(5,7-difluoro-6-(3-hydroxyprop-1-yn-1-yl)-4-oxo-1,4-dihydroquinolin-2-yl)-4-(methylsulfonyl)benzonitrile

A mixture of N-(2-acetyl-3,5-difluoro-4-(3-hydroxyprop-1-yn-1-yl)phenyl)-5-cyano-2-(methylsulfonyl)benzamide (15 mg, 0.03 mmol) and LiOtBu (4 mug, 0.05 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with ACN/20 (0-8%) to afford the title compound (1.5 mg) as an off-white solid. LCMS calc. for C₂₀H₁₁F₂N₂O₄S [M−H]⁻: m/z=413.1; Found: 413.0.

### Example 4: (R)-3-(5,7-Difluoro-6-(3-hydroxybut-1-yn-1-yl)-4-oxo-1,4-dihydroquinolin-2-yl)-4-(methylsulfonyl)benzonitrile

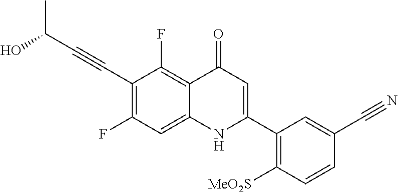 [SMILES: C[C@@H](O)C#Cc1c(F)cc2[nH]c(-c3cc(C#N)ccc3S(C)(=O)=O)cc(=O)c2c1F]

A mixture of CuI (0.4 mg 0.002 mmol), PdCl₂(PPh₃)₂ (2.8 mg 0.004 mmol), 3-(5,7-difluoro-6-iodo-4-oxo-1,4-dihydroquinolin-2-yl)-4-(methylsulfonyl)benzonitrile (20 mg, 0.04 mmol, Example 1 Step 5), and (R)-but-3-yn-2-ol (4.2 mg 0.06 mmol) in DMF (0.4 ml) and TEA (0.2 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (10-25%) to afford the title compound (5.2 mg) as an off-white solid. LCMS calc. for C₂₁H₁₅F₂N₂O₄S [M+H]⁺: m/z=429.1; Found: 429.1.

### Example 5: (S)-3-(5,7-Difluoro-6-(3-hydroxybut-1-yn-1-yl)-4-oxo-1,4-dihydroquinolin-2-yl)-4-(methylsulfonyl)benzonitrile

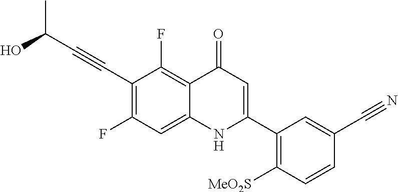 [SMILES: C[C@H](O)C#Cc1c(F)cc2[nH]c(-c3cc(C#N)ccc3S(C)(=O)=O)cc(=O)c2c1F]

This compound was prepared using procedures analogous to those described for Example 4 using (S)-but-3-yn-2-ol to replace (R)-but-3-yn-2-ol. LCMS calc. for C₂₁H₁₅F₂N₂O₄S [M+H]⁺: m/z=429.1; Found: 429.1.

### Example 6: 3-(5,7-Difluoro-6-(4-hydroxybut-1-yn-1-yl)-4-oxo-1,4-dihydroquinolin-2-yl)-4-(methylsulfonyl)benzonitrile

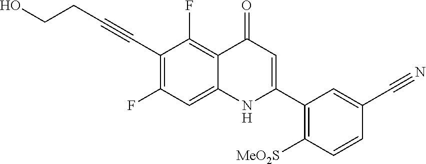 [SMILES: CS(=O)(=O)c1ccc(C#N)cc1-c1cc(=O)c2c(F)c(C#CCCO)c(F)cc2[nH]1]

This compound was prepared using procedures analogous to those described for Example 4 using but-3-yn-1-ol to replace (R)-but-3-yn-2-ol. LCMS calc. for C₂₁H₁₅F₂N₂O₄S [M+H]⁺: m/z=429.1; Found: 429.1.

### Example 7: 3-(5,7-Difluoro-6-((3-hydroxyoxetan-3-yl)ethynyl)-4-oxo-1,4-dihydroquinolin-2-yl)-4-(methylsulfonyl)benzonitrile

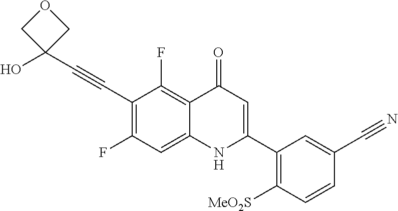 [SMILES: CS(=O)(=O)c1ccc(C#N)cc1-c1cc(=O)c2c(F)c(C#CC3(O)COC3)c(F)cc2[nH]1]

This compound was prepared using procedures analogous to those described for Example 4 using 3-ethynyloxetan-3-ol to replace (R)-but-3-yn-2-ol. LCMS calc. for C₂₂H₁₅F₂N₂O₅S [M+H]⁺. m/z=457.1; Found: 457.1.

### Example 8: 3-(6-(3-Amino-3-methylbut-1-yn-1-yl)-5,7-difluoro-4-oxo-1,4-dihydroquinolin-2-yl)-4-(methylsulfonyl)benzonitrile

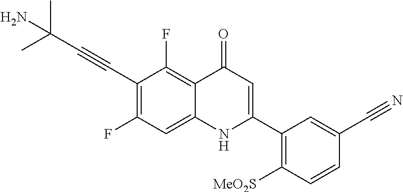 [SMILES: CC(C)(N)C#Cc1c(F)cc2[nH]c(-c3cc(C#N)ccc3S(C)(=O)=O)cc(=O)c2c1F]

This compound was prepared using procedures analogous to those described for Example 4 using 2-methylbut-3-yn-2-amine to replace (R)-but-3-yn-2-ol. LCMS calc. for C₂₂H₁₈F₂N₃O₃S [M+H]⁺: m/z=442.1; Found: 442.1.

### Example 9: 3-(6-(Cyclopropylethynyl)-5,7-difluoro-4-oxo-1,4-dihydroquinolin-2-yl)-4-(methylsulfonyl)benzonitrile

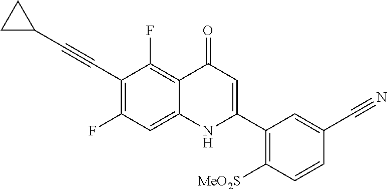 [SMILES: CS(=O)(=O)c1ccc(C#N)cc1-c1cc(=O)c2c(F)c(C#CC3CC3)c(F)cc2[nH]1]

### Step 1: N-(2-acetyl-4-(cyclopropylethynyl)-3,5-difluorophenyl)-5-cyano-2-(methylsulfonyl)benzamide

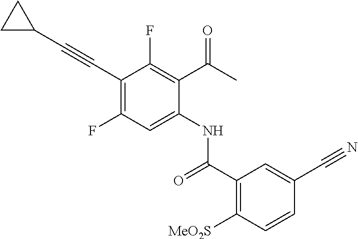 [SMILES: CC(=O)c1c(NC(=O)c2cc(C#N)ccc2S(C)(=O)=O)cc(F)c(C#CC2CC2)c1F]

A mixture of ethynylcyclopropane (20 mg, 0.3 mmol), N-(2-acetyl-3,5-difluoro-4-iodophenyl)-5-cyano-2-(methylsulfonyl)benzamide (100 mg, 0.2 mmol, Example 1 Step 4), CuI (4 mg, 0.02 mmol) and PdCl₂(PPh₃)₂ (14 mg, 0.02 mmol) in TEA (0.5 mL) and DMF (0.5 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (35-50%) to afford the title compound (28 mg) as an off-white solid. LCMS calc. for C₂₂H₁₇F₂N₂O₄S [M+H]⁺: m/z=443.1; Found: 433.0.

### Step 2: 3-(6-(cyclopropylethynyl)-5,7-difluoro-4-oxo-1,4-dihydroquinolin-2-yl)-4-(methylsulfonyl)benzonitrile

A mixture of N-(2-acetyl-4-(cyclopropylethynyl)-3,5-difluorophenyl)-5-cyano-2-(methylsulfonyl)benzamide (28 mg, 0.06 mmol) and LiOtBu (8 mg, 0.1 mmol) in 2-methyltetrahydrofuran (1 mL) was stirred at 75° C. overnight. The reaction was quenched with aq. HCl (0.1 mL, 2 M) and concentrated under reduced pressure. The residue was purified by flash chromatography on a C18 column eluting with ACN/H₂O (30-50%) to afford the title compound (3 mg) as an off-white solid. LCMS calc. for C₂₂H₁₃F₂N₂O₃S [M−H]⁻: m/z=425.1; Found: 425.1.

### Example 10: 3-(6-(3,3-Dimethylbut-1-yn-1-yl)-5,7-difluoro-4-oxo-1,4-dihydroquinolin-2-yl)-4-(methylsulfonyl)benzonitrile

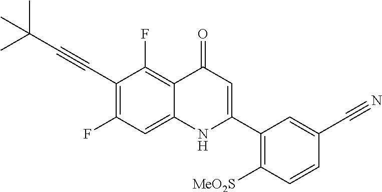 [SMILES: CC(C)(C)C#Cc1c(F)cc2[nH]c(-c3cc(C#N)ccc3S(C)(=O)=O)cc(=O)c2c1F]

This compound was prepared using procedures analogous to those described for Example 9 Step 1-2 using 3,3-dimethylbut-1-yne to replace ethynylcyclopropane in Step 1. LCMS calc. for C₂₃H₁₉F₂N₂O₃S [M+H]⁺: m/z=441.1; Found: 441.1.

### Example 11: 3-(5,7-Difluoro-4-oxo-6-(phenylethynyl)-1,4-dihydroquinolin-2-yl)-4-(methylsulfonyl)benzonitrile

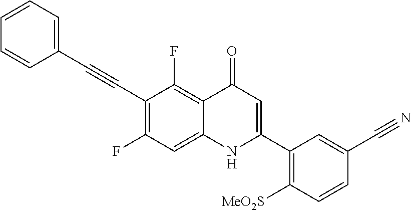 [SMILES: CS(=O)(=O)c1ccc(C#N)cc1-c1cc(=O)c2c(F)c(C#Cc3ccccc3)c(F)cc2[nH]1]

This compound was prepared using procedures analogous to those described for Example 9 Step 1-2 using ethynylbenzene to replace ethynylcyclopropane in Step 1. LCMS calc. for C₂₅H₁₅F₂N₂O₃S [M+H]⁺: m/z=460.1; Found: 460.1.

### Example 12: 3-(5,7-Difluoro-6-((1-methyl-1H-pyrazol-4-yl)ethynyl)-4-oxo-1,4-dihydroquinolin-2-yl)-4-(methylsulfonyl)benzonitrile

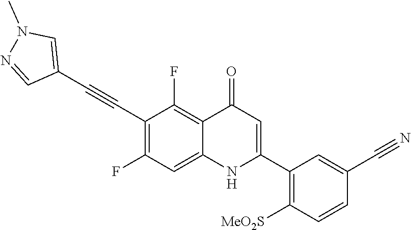 [SMILES: Cn1cc(C#Cc2c(F)cc3[nH]c(-c4cc(C#N)ccc4S(C)(=O)=O)cc(=O)c3c2F)cn1]

This compound was prepared using procedures analogous to those described for Example 4 using 4-ethynyl-1-methyl-1H-pyrazole to replace (R)-but-3-yn-2-ol. LCMS calc. for C₂₃H₁₅F₂N₄O₃S [M+H]⁺: m/z=465.1; Found: 465.1.

### Example 13: 3-(6-Cyclopropyl-5,7-difluoro-4-oxo-1,4-dihydroquinolin-2-yl)-4-(methylsulfonyl)benzonitrile

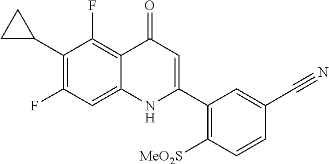 [SMILES: CS(=O)(=O)c1ccc(C#N)cc1-c1cc(=O)c2c(F)c(C3CC3)c(F)cc2[nH]1]

### Step 1: N-(2-acetyl-4-cyclopropyl-3,5-difluorophenyl)-5-cyano-2-(methylsulfonyl)benzamide

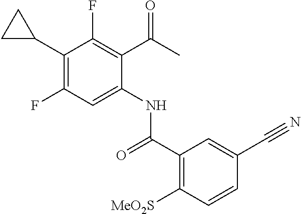 [SMILES: CC(=O)c1c(NC(=O)c2cc(C#N)ccc2S(C)(=O)=O)cc(F)c(C2CC2)c1F]

A mixture of cyclopropylboronic acid (26 mg, 0.3 mmol), N-(2-acetyl-3,5-difluoro-4-iodophenyl)-5-cyano-2-(methylsulfonyl)benzamide (50 mg, 0.1 mmol, Example 1 Step 4), Pd(dppf)Cl₂ (7 mg, 0.01 mmol) and K₂CO₃ (68 mg, 0.5 mmol) in 1,4-dioxane (1 mL) and water (0.1 mL) was degassed and recharged with nitrogen for 3 cycles, and then stirred at 100° C. overnight. The resulting mixture was concentrated under reduced pressure. The residue was purified by prep-TLC eluting with EtOAc/PE (20%) to afford the title compound (18 mg) as a yellow oil. LCMS calc. for C₂₀H₁₇F₂N₂O₄S [M+H]⁺: m/z=419.1; Found: 419.1.

### Step 2: 3-(6-cyclopropyl-5,7-difluoro-4-oxo-1,4-dihydroquinolin-2-yl)-4-(methylsulfonyl)benzonitrile

A mixture of N-(2-acetyl-4-cyclopropyl-3,5-difluorophenyl)-5-cyano-2-(methylsulfonyl)benzamide (18 mg, 0.04 mmol) and LiOtBu (4 tug, 0.05 mmol) in 2-methyltetrahydrofuran (0.5 mL) was stirred at 75° C. overnight. The reaction was quenched with aq. HCl (0.1 mL, 2 M) and concentrated under reduced pressure. The residue was purified by flash chromatography on a C18 column eluting with ACN/H₂O (20-40%) to afford the title compound (1.4 mg) as an off-white solid. LCMS calc. for C₂₀H₁₅F₂N₂O₃S [M+H]⁺: m/z=401.1; Found: 401.1.

### Example 14: 4-Fluoro-3-(4-fluoro-1-methyl-5-oxo-5,8-dihydro-1H-pyrazolo[4,3-g]quinolin-7-yl)benzonitrile

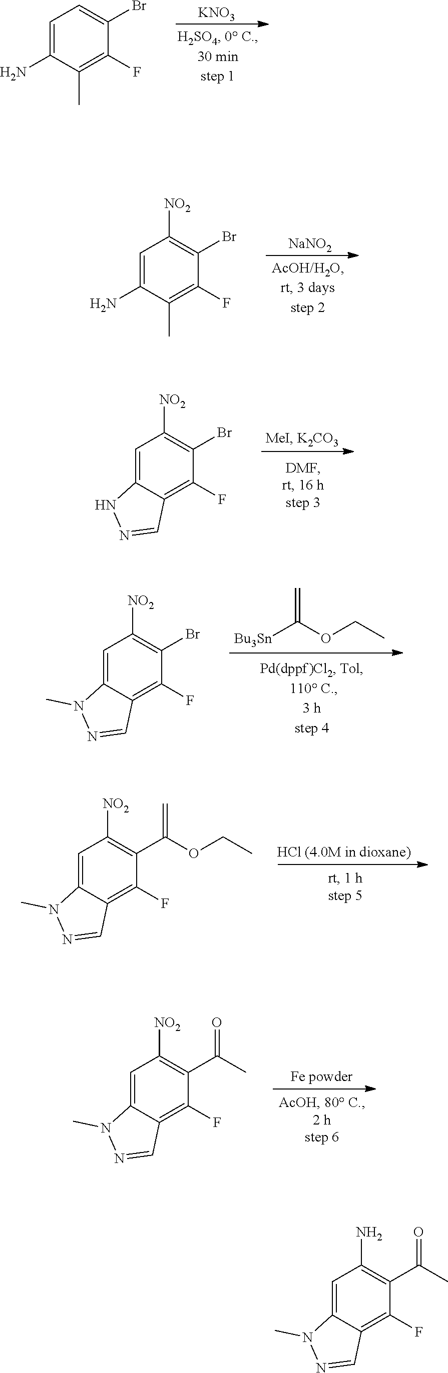

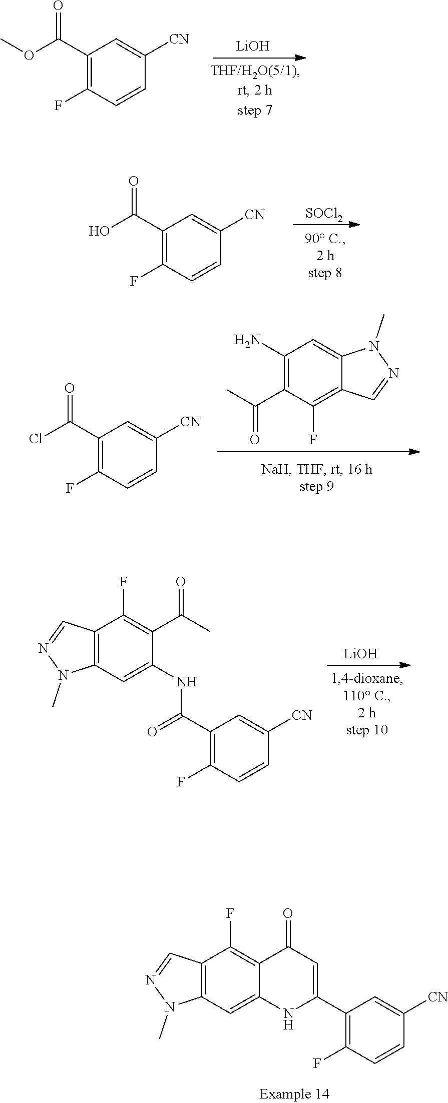

### Step 1: 4-bromo-3-fluoro-2-methyl-5-nitroaniline

To a solution of 4-bromo-3-fluoro-2-methylaniline (10 g, 49.3 mol) in H₂SO₄ (150 mL) was added KNO₃ (6 g, 60 mmol) 0-10° C. The reaction mixture was stirred for 30 min. at 0-10° C. Then the resulting mixture was poured into crushed ice, filtered, and concentrated under reduced pressure to afford the title compound (12 g, 98% yield) as a yellow solid. LCMS calc. for C₇H₇BrFN₂O₂ [M+H]⁺: m/z=248.9; Found: 248.9, 250.8.

### Step 2: 5-bromo-4-fluoro-6-nitro-1H-indazole

To a solution of 4-bromo-3-fluoro-2-methyl-5-nitroaniline (12 g, 48.4 mmol) in AcOH (500 mL) was added NaNO₂ (3.4 g, 48.4 mmol) in H₂O (10 mL), and the mixture was stirred at r.t. for 3 days. Then the reaction mixture was concentrated under reduced pressure. To the residue was added 100 mL water and stirred. The formed solid was collected by filtration, washed with water, and dried. The crude product was recrystallized in ethanol and water to give the title compound (10 g, 80% yield) as a yellow solid. LCMS calc. for C₇H₄BrFN₃O₂[M+H]⁺: m/z=259.9; Found: 260.1, 262.1.

### Step 3: 5-bromo-4-fluoro-1-methyl-6-nitro-1H-indazole

A mixture of 5-bromo-4-fluoro-6-nitro-1H-indazole (10 g, 38.6 mmol), iodomethane (6 g, 42.5 mmol) and K₂CO₃ (16 g, 115.8 mmol) in DMF (300 mL) was stirred at r.t. overnight. The mixture was diluted with H₂O (300 mL) and extracted with DCM (200 mL×2). The combined organic layer was washed with brine (300 mL) and dried over Na₂SO₄, filtered and concentrated under reduced pressure. The residue was purified by flash chromatography on a silica gel column eluting with DCM/PE (50%) to afford the title compound (4.2 g, 40% yield) as a yellow solid. LCMS calc. for C₈H₆BrFN₃O₂ [MH]⁺: m/z=273.9; Found: 274.0, 275.8.

### Step 4: 5-(1-ethoxyvinyl)-4-fluoro-1-methyl-6-nitro-1H-indazole

A mixture of 5-bromo-4-fluoro-1-methyl-6-nitro-1H-indazole (2 g, 7.3 mmol) tributyl(1-ethoxyvinyl)stannane (2.9 g, 8.1 mmol) and 1,1′-Bis(diphenylphosphino)ferrocene-palladium(II)dichloride dichloromethane complex (598 mg, 0.73 mmol) in toluene (25 mL) was stirred at 110° C. for 3 h. The mixture was used next step without further work-up. LCMS calc. for C₁₂H₁₃FN₃O₃ [M+H]⁺: m/z=266.1; Found: 266.3.

### Step 5: 1-(4-fluoro-1-methyl-6-nitro-1H-indazol-5-yl)ethan-1-one

To the mixture above was added 10 mL HCl (4.0 M in dioxane) and stirred at r.t. for 1 h. The mixture was diluted with H₂O (100 mL) and extracted with DCM (20 mL×3) and the combined organic layer was washed with brine (30 mL) and dried over Na₂SO₄, filtered and concentrated under reduced pressure. The residue was purified by flash chromatography on a silica gel column eluting with DCM/PE (50%) to afford the title compound (1.2 g, 69% yield) as a yellow solid. LCMS calc. for C₁₀H₉FN₃O₃ [M+H]⁺. m/z=238.0; Found: 238.0.

### Step 6: 1-(6-amino-4-fluoro-1-methyl-1H-indazol-5-yl)ethan-1-one

To a solution of 1-(4-fluoro-1-methyl-6-nitro-1H-indazol-5-yl)ethan-1-one (800 mg, 3.4 mmol) in AcOH (20 mL) was added Fe powder (940 mg, 16.8 mmol), and the mixture was stirred at r.t. for 2 h. The reaction mixture was filtered, the filter cake was washed with DCM, the filtrate was concentrated under reduced pressure. The residue was diluted with H₂O (20 mL) and extracted with DCM (20 mL×3) and the combined organic layer was washed with brine (30 mL) and dried over Na₂SO₄, filtered and concentrated to afford the title compound (700 mg, 98% yield) as a yellow solid.

¹H NMR (400 MHz, DMSO-d₆) δ 8.01 (d, J=1.0 Hz, 1H), 6.91 (s, 2H), 6.40 (d, J=1.0 Hz, 1H), 3.81 (s, 3H), 2.58 (d, J=8.3 Hz, 3H). LCMS calc. for C₁₀H₁₁FN₃O [M+H]⁺: m/z=208.1; Found: 208.0.

### Step 7: 5-cyano-2-fluorobenzoic acid

A mixture of methyl 5-cyano-2-fluorobenzoate (2 g, 11.17 mmol), LiOH (295 mg, 12.29 mol), THF (25 mL) and water (5 mL) was stirred at r.t. for 2 h. The solution was acidified with conc. HCl (12 M) and concentrated under reduced pressure to afford crude 5-cyano-2-fluorobenzoic acid (1.3 g, 7.88 mmol, 72% yield) as a white solid, which was directly used in the next step without further purification. ¹H NMR (400 MHz, DMSO-d₆) δ 13.77 (s, 1H), 8.30 (dd, J=6.7, 2.2 Hz, 1H), 8.15 (ddd, J=8.6, 4.4, 2.3 Hz, 1H), 7.58 (dd, J=10.3, 8.9 Hz, 1H).

### Step 8: 5-cyano-2-fluorobenzoyl chloride

A solution of 5-cyano-2-fluorobenzoic acid (500 mg, 3.03 mmol) in thionyl chloride (3606 mg, 30.3 mmol) was heated at 90° C. for 2 h. The solution was cooled, concentrated, diluted with toluene, and concentrated to afford 2-fluoro-5-cyanobenzoyl chloride which was used in the next step without further purification.

### Step 9: N-(5-acetyl-4-fluoro-1-methyl-1H-indazol-6-yl)-5-cyano-2-fluorobenzamide

To a solution of 1-(6-amino-4-fluoro-1-methyl-1H-indazol-5-yl)ethan-1-one (500 mg, 2.42 mmol) in THF (12 mL) was added NaH (97 mg, 2.42 mmol, 60% suspension in mineral oil) at 0° C. After stirring for 30 min., a solution of 5-cyano-2-fluorobenzoyl chloride (487 mg, 2.66 mmol) in THF (10 mL) was dropwise added to the mixture above. The resulting mixture was stirred at r.t. for 16 h. Then the reaction mixture was quenched with saturated NH₄Cl aq. (5 mL) at r.t., then diluted with water (10 mL), and filtered. The filter cake was triturated with EtOAc (20 mL) and filtered to afford the title compound (705 mg, 82% yield) as a yellow solid. LCMS calc. for C₁₈H₁₃F₂N₄O₂ [M+H]⁺: m/z=355.1; Found: 355.1

### Step 10: 4-fluoro-3-(4-fluoro-1-methyl-5-oxo-5,8-dihydro-1H-pyrazolo[4,3-g]quinolin-7-yl)benzonitrile

A mixture of N-(5-acetyl-4-fluoro-1-methyl-1H-indazol-6-yl)-5-cyano-2-fluoro benzamide (600 mg, 1.69 mmol), LiGH (81 mg, 3.38 mmol) in dioxane (7 mL) was stirred at 110° C. for 1.5 h. The reaction mixture was adjusted to pH~5 with HCl aq. (1 M) and then diluted with water (30 mL) and extracted with EtOAc (100 mL×3). The combined organic layers were washed with brine (100 mL×2), dried over Na₂SO₄, filtered, and concentrated under reduced pressure. The residue was purified by flash chromatography on a silica gel column eluting with MeOH/DCM (5%) to afford the title compound (509 mg, 1.51 mmol, 89% yield) as a yellow solid. ¹H NMR (400 MHz, DMSO-d₆) δ 11.79 (s, 1H), 8.38 (d, J=0.9 Hz, 1H), 8.33 (dd, J=6.8, 2.1 Hz, 1H), 8.16 (ddd, J=8.6, 4.7, 2.2 Hz, 1H), 7.71 (dd, J=10.1, 8.7 Hz, 1H), 7.38 (s, 1H), 6.05 (s, 1H), 4.05 (s, 3H). LCMS calc. for C₁₈H₁₁F₂N₄O [M+H]⁺: m/z=337.1; Found: 337.1.

### Example 15: 3-(4-Fluoro-1-methyl-5-oxo-5,8-dihydro-1H-pyrazolo[4,3-g]quinolin-7-yl)-4-(methylthio)benzonitrile

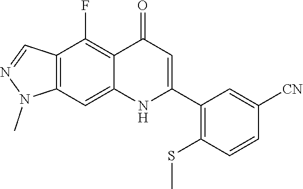 [SMILES: CSc1ccc(C#N)cc1-c1cc(=O)c2c(F)c3cnn(C)c3cc2[nH]1]

A mixture of 4-fluoro-3-(4-fluoro-1-methyl-5-oxo-5,8-dihydro-1H-pyrazolo[4,3-g]quinolin-7-yl)benzonitrile (100 mg, 0.30 mmol, Example 14), sodium methanethiolate (42 mg, 0.60 mmol) in NMP (1 mL) was stirred at r.t. for 3 h. To the mixture was added EtOAc (10 mL) and washed with H₂O (2×10 mL) and brine (2×10 mL). The organic phase was dried over Na₂SO₄, filtered and concentrated under reduced pressure. The residue was purified by Prep-HPLC on a C18 column eluting with MeCN/H₂O (5%-95%, with 5% FA) to afford the title compound (50 mg, 46% yield) as a yellow solid. ¹H NMR (400 MHz, DMSO-d₆) δ 11.77 (s, 1H), 8.38 (s, 1H), 7.98-7.97 (m, 2H), 7.60 (d, J=9.1 Hz, 1H), 7.32 (s, 11H), 5.83 (d, J=1.6 Hz, 1H), 4.03 (s, 3H), 2.54 (s, 3H). LCMS calc. for C₁₉H₁₄FN₄OS [M+H]⁺: m/z=365.4; Found: 365.3.

### Example 16: 3-(4-Fluoro-1-methyl-5-oxo-5,8-dihydro-1H-pyrazolo[4,3-g]quinolin-7-yl)-4-(methylsulfonyl)benzonitrile

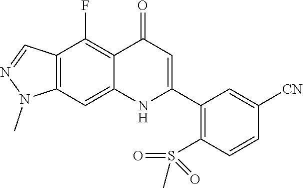 [SMILES: Cn1ncc2c(F)c3c(=O)cc(-c4cc(C#N)ccc4S(C)(=O)=O)[nH]c3cc21]

To a solution of 3-(4-fluoro-1-methyl-5-oxo-5,8-dihydro-11H-pyrazolo[4,3-g]quinolin-7-yl)-4-(methylthio)benzonitrile (50 mg, 0.15 mmol, Example 15) in acetone (0.5 mL), H₂O (0.3 mL), THE (0.36 mL) and MeOH (0.36 mL) was added oxone (461 mg, 0.75 mmol). The mixture was stirred at 50° C. for 3 h. The reaction mixture was cooled to r.t. and quenched with Na₂SO₃ aq. The reaction mixture was diluted with H₂O (2 mL) and filtered. The filter cake was washed with water and dried under reduced pressure. Then the residue was purified by Prep-HPLC on a C18 column eluting with MeCN/H₂O (5%-95%, with 0.05% NH₄HCO₃) to afford the title compound (12.1 mg, 20% yield) as a light red solid. ¹H NMR (400 MHz, DMSO-d₆) δ 11.91 (s, 1H), 8.35 (m, 4H), 7.26 (s, 1H), 5.95 (d, J=1.2 Hz, 1H), 4.04 (s, 3H), 3.31 (s, 3H). LCMS calc. for C₁₉H₁₄FN₄O₃S [M+H]⁺: m/z=397.1; found: 396.8.

### Example 17: 3-(5,7-Difluoro-4-oxo-6-(o-tolylethynyl)-1,4-dihydroquinolin-2-yl)-4-(methylsulfonyl)benzonitrile

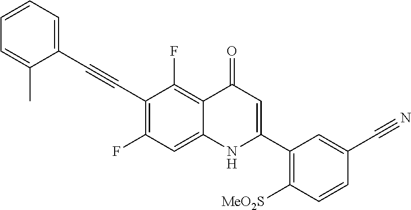 [SMILES: Cc1ccccc1C#Cc1c(F)cc2[nH]c(-c3cc(C#N)ccc3S(C)(=O)=O)cc(=O)c2c1F]

### Step 1: N-(2-acetyl-3,5-difluoro-4-(o-tolylethynyl)phenyl)-5-cyano-2-(methylsulfonyl)benzamide

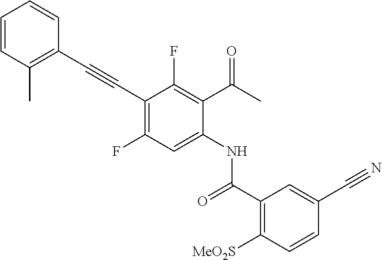 [SMILES: CC(=O)c1c(NC(=O)c2cc(C#N)ccc2S(C)(=O)=O)cc(F)c(C#Cc2ccccc2C)c1F]

To a mixture of 1-ethynyl-2-methylbenzene (35 mg, 0.3 mmol), N-(2-acetyl-3,5-difluoro-4-iodophenyl)-5-cyano-2-(methylsulfonyl)benzamide (100 mg, 0.2 mmol, Example 1 Step 4), CuI (4 mg, 0.02 mmol) and PdCl₂(PPh₃)₂ (14 mg, 0.02 mmol) in TEA (0.5 mL) and DMF (0.5 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (35-55%) to afford the title compound (54 mg) as an off-white solid. LCMS calc. for C₂₆H₁₉F₂N₂O₄S [M+H]⁺: m/z=493.1; Found: 493.1.

### Step 2: 3-(5,7-difluoro-4-oxo-6-(o-tolylethynyl)-1,4-dihydroquinolin-2-yl)-4-(methylsulfonyl)benzonitrile

A mixture of N-(2-acetyl-3,5-difluoro-4-(o-tolylethynyl)phenyl)-5-cyano-2-(methylsulfonyl)benzamide (54 mg, 0.11 mmol) and LiOtBu (17.6 mg, 0.22 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with ACN/H₂O (35-55% with 0.1 NH₄CO₃) to afford the title compound (3.2 mg) as a brown solid. LCMS calc. for C₂₆H₁₅F₂N₂O₃S [M−H]⁻: m/z=473.1; Found: 473.1.

### Example 18: 3-(6-(Benzyloxy)-5,7-difluoro-4-oxo-1,4-dihydroquinolin-2-yl)-4-(methylsulfonyl)benzonitrile

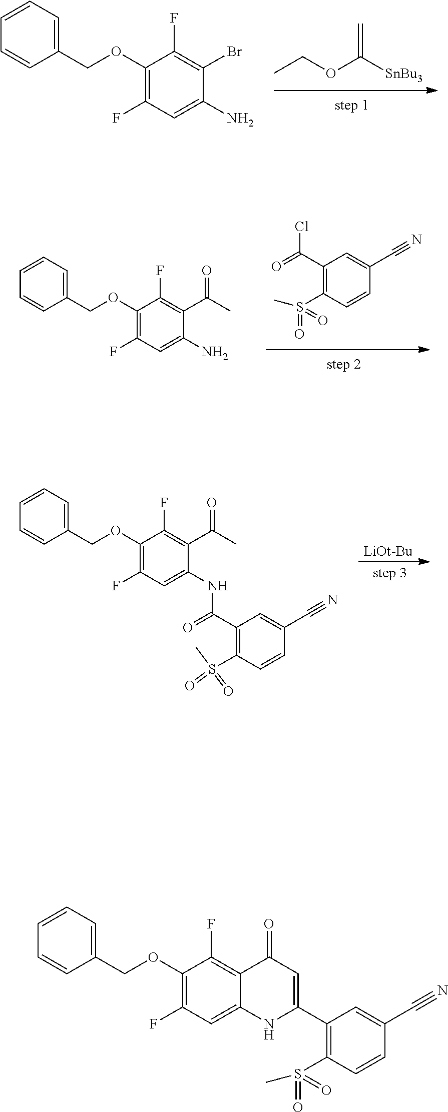

### Step 1: 1-(6-amino-3-(benzyloxy)-2,4-difluorophenyl)ethan-1-one

A mixture of 4-(benzyloxy)-2-bromo-3,5-difluoroaniline (250.0 mg, 0.79 mmol), tributyl(1-ethoxyvinyl)stannane (429 mg, 1.19 mmol) and Pd(PPh₃)₄ (91 mug, 0.079 mmol) in 1,4-dioxane was degassed and charged with N₂ and stirred at 100° C. for 18 h. The reaction mixture was concentrated under reduced pressure to give a crude intermediate which was dissolved into acetone (2 mL) and 2M HCl (1 mL). The resulting mixture was stirred at r.t, for 2 h. The mixture was quenched with water (10 mL) and extracted with EA (30 mL×3). The combined organic layers were washed with NaHCO₃ aqueous, brine, dried over Na₂SO₄, filtered and concentrated under reduced pressure. The residue was purified by flash chromatography on a silica gel column eluting with PE/EA (15%) to afford the title compound (110 mg, 75.6% yield) as a yellow solid. LCMS calc. for C₁₅H₁₄F₂NO₂ [M+H]⁺: m/z=278.0; Found: 278.0

### Step 2: N-(2-acetyl-4-(benzyloxy)-3,5-difluorophenyl)-5-cyano-2-(methylsulfonyl)benzamide

A mixture of 1-(6-amino-3-(benzyloxy)-2,4-difluorophenyl)ethan-1-one (80.0 mg, 0.29 mmol) in isopropyl acetate (1 mL) was added 5-cyano-2-(methylsulfonyl)benzoyl chloride (91 mg, 0.375 mmol) charged with N₂ and stirred at 80° C. for 18 h. The mixture was quenched with water (10 mL) and extracted with EA (20 mL×3). The combined organic layers were washed with NaHCO₃ aqueous, brine, dried over Na₂SO₄, filtered and concentrated under reduced. The residue was purified by flash chromatography on a silica gel column eluting with PE/EA (20%) to afford the title compound (80 mg, 57% yield) as a white solid. LCMS calc. for C₂₄H₁₉F₂N₂O₅S [M+H]⁺: m/z=485.1; Found: 485.1.

### Step 3: 3-(6-(benzyloxy)-5,7-difluoro-4-oxo-1,4-dihydroquinolin-2-yl)-4-(methylsulfonyl)benzonitrile

To a solution of N-(2-acetyl-4-(benzyloxy)-3,5-difluorophenyl)-5-cyano-2-(methylsulfonyl)benzamide (80 mg, 0.165 mmol) in 2-methyltetrahydrofuran (1 mL) was added LiOtBu (21 mg, 0.264 mmol) at 0° C. under N₂. The mixture was stirred at 75° C. overnight. The reaction was quenched with aq. HCl (0.1 mL, 2 M). The organic layers were washed with brine, dried over Na₂SO₄, filtered and concentrated tinder reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 mL/min Gradient: 30-60 Detection wavelength: 214/254 nm Retention time: 6-14 min) to afford the title compound (2.16 mg, 2.8% yield) as a white solid. ¹H NMR (400 MHz, DMSO-d₆) δ 12.05 (s, 1H), 8.27 (d, J=7.8 Hz, 3H), 7.62-7.33 (m, 5H), 7.12 (s, 1H), 6.08 (s, 1H), 5.19 (s, 2H), 3.29 (s, 3H). LCMS calc. for C₂₄H₁₇F₂N₂O₄S [M+H]⁺: m/z=467.1; Found: 467.3.

### Example 19: 3-(5,7-Difluoro-4-oxo-6-(prop-1-yn-1-yl)-1,4-dihydroquinolin-2-yl)-4-(methylsulfonyl)benzonitrile

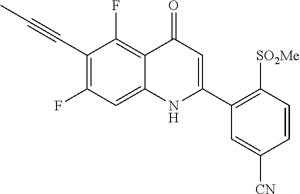 [SMILES: CC#Cc1c(F)cc2[nH]c(-c3cc(C#N)ccc3S(C)(=O)=O)cc(=O)c2c1F]

### Step 1: 3-(5,7-difluoro-4-Oxo-6-(3-(trimethylsilyl)prop-1-yn-1-yl)-1,4-dihydroquinolin-2-yl)-4-(methylsulfonyl)benzonitrile

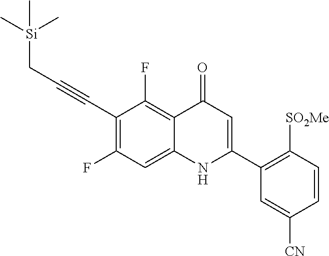 [SMILES: C[Si](C)(C)CC#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (100 mg, 0.2 mmol, Example 1 Step 5), trimethyl(prop-2-yn-1-yl)silane (35.0 mg, 0.31 mmol), CuI (4.0 mg, 0.02 mmol) and PdCl₂(PPh₃)₂ (15.0 mg, 0.02 mmol) in TEA (0.5 mL) and DMF (2 mL) was degassed and recharged with nitrogen for 3 cycles, and then stirred at 60° C. for 2.5 h. The reaction mixture was diluted with water (10 mL) and extracted with EA (10 mL×2). The combined organic layers were washed with brine, dried over Na₂SO₄ and filtered. The filtrate was concentrated under reduced pressure. The residue was purified by prep-TLC(DCM/MeOH=20/1) to afford the title compound (45 mg, 47.8% yield) as a white solid. LCMS calc. for C₂₃H₂₁F₂N₂O₃SSi [M+H]⁺: m/z=471.1; Found: 471.1.

### Step 2: 3-(5,7-difluoro-4-oxo-6-(prop-1-yn-1-yl)-1,4-dihydroquinolin-2-yl)-4-(methylsulfonyl)benzonitrile

The mixture of 3-(5,7-difluoro-4-oxo-6-(3-(trimethylsilyl)prop-1-yn-1-yl)-1,4-dihydroquinolin-2-yl)-4-(methylsulfonyl)benzonitrile (50 mg, 0.06 mmol) in THF/H₂O (3 mL/0.3 mL) was added KOH (10.7 g, 0.19 mmol). The reaction mixture was stirred at r.t. for 4 h. The reaction mixture was concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 mL/min Gradient: 30-60 Detection wavelength: 214/254 nm Retention time: 6-14 min) to afford the title compound (3.1 mg, 12.9% yield) as a white solid. ¹H NMR (400 MHz, DMSO-d₆) δ 12.22 (s, 11H), 8.35-8.21 (m, 3H), 7.18 (s, 1H), 6.24 (s, 11H), 3.34 (s, 3H), 2.17 (s, 3H). LCMS calc. for C₂₀H₁₃F₂N₂O₃S [M+H]⁺: m/z=399.1; Found: 399.1.

### Example 20: 3-(6-(But-1-yn-1-yl)-5,7-difluoro-4-oxo-1,4-dihydroquinolin-2-yl)-4-(methylsulfonyl)benzonitrile

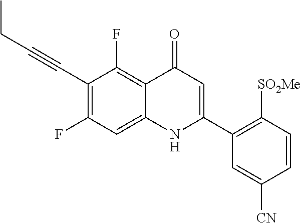 [SMILES: CCC#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 2-pentynoic acid (36 mg, 0.370 mmol), CuI (2.34 mg, 0.0123 mmol), PPh₃ (3.2 mg, 0.0123 mmol) and Pd(OAc)₂ (2.8 mg, 0.0123 mmol) in TEA (0.1 mL) and DMF (1 mL) was degassed and recharged with nitrogen for 3 cycles, and then stirred at 50° C. for 16 h. The reaction mixture was diluted with water (10 mL) and extracted with EA (10 mL×2). The combined organic layers were washed with brine, dried over Na₂SO₄ and filtered. The filtrate was concentrated under reduced pressure. The residue was purified by prep-HPLC (Instrument: GILSON-281 Column: XBridge C18 250*19.00 mml 10 urn Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (18.4 mg, 36.3% yield) as a yellow solid. ¹H NMR (400 MHz, DMSO-d₆) δ 12.22 (s, 1H), 8.42-8.18 (m, 3H), 7.12 (d, J=9.1 Hz, 1H), 6.16 (s, 1H), 3.30 (s, 3H), 2.58-2.52 (m, 2H), 1.21 (dd, J=12.8, 5.4 Hz, 3H). LCMS calc. for C₂₁H₁₅F₂N₂O₃S [M+H]⁺: m/z=413.4; Found: 413.4.

### Example 21: 3-(5,7-Difluoro-6-(3-methylbut-1-yn-1-yl)-4-oxo-1,4-dihydroquinolin-2-yl)-4-(methylsulfonyl)benzonitrile

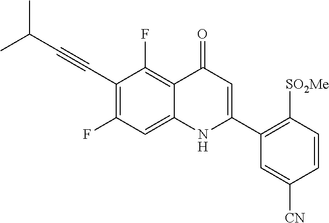 [SMILES: CC(C)C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (50 mg, 0,103 mmol, Example 1 Step 5), 3-methylbut-1-yne (10.5 mg, 0.154 mmol), CuI (2 mg, 0.0103 mmol) and PdCl₂(PPh₃)₂ (7.23 mg, 0.0103 mmol) in TEA (0.3 mL) and DMF (1 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (6.3 mg, 14.34% yield) as a white solid. ¹H NMR (400 MHz, DMSO-d₆) δ 12.24 (s, 1H), 8.42-8.18 (m, 3H), 7.11 (d, J=10.0 Hz, 1H), 6.16 (s, 1H), 3.30 (s, 3H), 3.01-2.86 (m, 1H), 1.25 (d, J=6.8 Hz, 6H). LCMS calc. for C₂₂H₁₇F₂N₂O₃S [M+H]⁺: m/z=427.1; Found: 427.2.

### Example 22: 3-(5,7-Difluoro-4-oxo-6-(pyridin-4-ylethynyl)-1,4-dihydroquinolin-2-yl)-4-(methylsulfonyl)benzonitrile

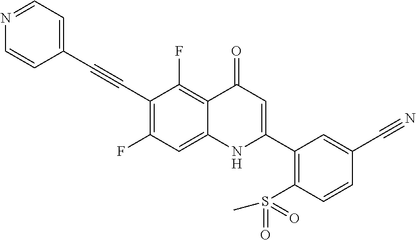 [SMILES: CS(=O)(=O)c1ccc(C#N)cc1-c1cc(=O)c2c(F)c(C#Cc3ccncc3)c(F)cc2[nH]1]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 4-ethynylpyridine (19 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 75° C. for 5 h. The reaction mixture was diluted with water (10 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford crude product. The crude product was washed with MeOH and dried in vacuum to afford the title compound (9.4 mg, 17% yield) as a yellow solid. ¹H NMR (400 MHz, DMSO-d₆) δ 12.38 (s, 1H), 8.68 (d, J=5.9 Hz, 2H), 8.41-8.23 (m, 3H), 7.58 (d, J=5.8 Hz, 2H), 7.22 (s, 1H), 6.23 (s, 1H), 3.33 (s, 3H). LCMS calc. for C₂₄H₁₄F₂N₃O₃S [M+H]⁺: m/z=462.1; Found: 462.1.

### Example 23: 3-(5,7-Difluoro-6-(3-fluoro-3-methylbut-1-yn-1-yl)-4-oxo-1,4-dihydroquinolin-2-yl)-4-(methylsulfonyl)benzonitrile

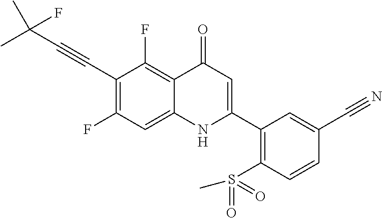 [SMILES: CC(C)(F)C#Cc1c(F)cc2[nH]c(-c3cc(C#N)ccc3S(C)(=O)=O)cc(=O)c2c1F]

To a solution of 3-(5,7-difluoro-6-(3-hydroxy-3-methylbut-1-yn-1-yl)-4-oxo-1,4-dihydroquinolin-2-yl)-4-(methylsulfonyl)benzonitrile (17.0 mg, 0.04 mmol, Example 1 Step 6) in DCM (2.5 mL) was added DAST (12.4 mg, 0.07 mmol) dropwise at 0° C. under N₂. The mixture was stirred at r.t. for 1 h. The reaction mixture was diluted with DCM (5 mL) and poured into ice water (10 mL), and extracted with DCM (10 mL×2). The combined organic layers were dried over Na₂SO₄, filtered and concentrated under reduced pressure. The residue was purified by flash chromatography on a silica gel column eluting with DCM/MeOH (2%) to afford the title compound (0.61 mg, 3.4% yield) as a white solid. ¹H NMR (400 MHz, DMSO-d₆) δ 12.32 (s, 1H), 8.45-8.17 (m, 3H), 7.16 (d, J=9.3 Hz, 1H), 6.19 (s, 1H), 3.29 (s, 3H), 1.77 (s, 3H), 1.72 (s, 3H). LCMS calc. for C₂₂H₁₆F₃N₂O₃S [M+H]⁺: m/z=445.1; Found: 445.1.

### Example 24: 3-(5,7-Difluoro-6-(oxetan-3-ylethynyl)-4-oxo-1,4-dihydroquinolin-2-yl)-4-(methylsulfonyl)benzonitrile

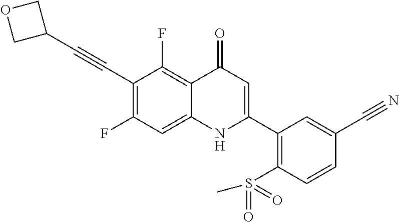 [SMILES: CS(=O)(=O)c1ccc(C#N)cc1-c1cc(=O)c2c(F)c(C#CC3COC3)c(F)cc2[nH]1]

A mixture of 3-(5,7-difluoro-6-iodo-4-oxo-1,4-dihydroquinolin-2-yl)-4-(methylsulfonyl)benzonitrile (30 mg, 0.062 mmol, Example 1 Step 5), 3-ethynyloxetane (7.6 mg, 0.093 mmol), CuI (1.18 mg, 0.0062 mmol) and PdCl₂(PPh₃)₂ (7.6 mg, 0.0062 mmol) in TEA (0.2 mL) and DMF (0.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (0.5 mg, 1.8% yield) as a white solid. ¹H NMR (400 MHz, DMSO) δ 12.29 (s, 1H), 8.30 (s, 3H), 7.16 (s, 1H), 6.18 (s, 1H), 4.85 (dd, J=8.5, 5.5 Hz, 2H), 4.66-4.62 (m, 2H), 4.29 (dd, J=15.5, 7.9 Hz, 1H), 3.30 (s, 3H). LCMS calc. for C₂₂H₁₅F₂N₂O₄S [M+H]⁺: m/z=441.1; Found: 441.1.

### Example 25: 3-(5,7-Difluoro-4-oxo-6-(2-(tetrahydro-2H-pyran-4-yl)ethyl)-1,4-dihydroquinolin-2-yl)-4-(methylsulfonyl)benzonitrile

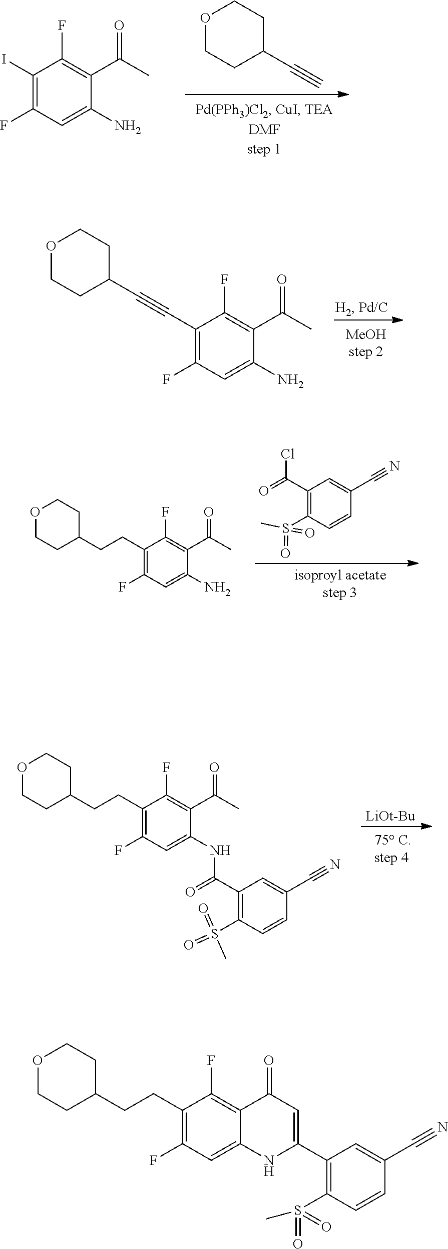

### Step 1: 1-(6-amino-2,4-difluoro-3-((tetrahydro-2H-pyran-4-yl)ethynyl)phenyl)ethan-1-one

A mixture of 1-(6-amino-2,4-difluoro-3-iodophenyl)ethan-1-one (500.0 mg, 1.68 mmol), 4-ethynyltetrahydro-2H-pyran (278.3 mg, 2.52 mmol), Pd(PPh₃)Cl₂ (117.9 mg, 0.168 mmol), CuI (31.9 mg, 0.168 mmol) and TEA (2 mL) in DMF (8 mL) was stirred at 60° C. for 2 h. under N₂. The mixture was concentrated under reduced pressure. The residue was purified by flash chromatography on a silica gel column eluting with PE/EA (15%) to afford the title compound (360 mg, 76.5% yield) as a yellow solid. LCMS calc. for C₁₅H₁₆F₂NO₂ [M+H]⁺: m/z=280.3; Found: 280.3.

### Step 2: 1-(6-amino-2,4-difluoro-3-(2-(tetrahydro-2H-pyran-4-yl)ethyl)phenyl)ethan-1-one

A mixture of 1-(6-amino-2,4-difluoro-3-((tetrahydro-2H-pyran-4-yl)ethynyl)phenyl)ethan-1-one (200.0 mg, 0.72 mmol) and Pd/C (10%, 200.0 mg) in MeOH (15 mL) was stirred at r.t. for 18 h. under H₂. The reaction mixture was diluted with MeOH (5 mL) and filtered by a pad of celite. The filtrate was concentrated under reduced pressure to afford the title compound (170.0 mg, 83.4% yield) as a white solid which was used in the next step without further purification. LCMS calc. for C₁₅H₂₀F₂NO₂ [M+H]⁺: m/z=284.2; Found: 284.2.

### Step 3: N-(2-acetyl-3,5-difluoro-4-(2-(tetrahydro-2H-pyran-4-yl)ethyl)phenyl)-5-cyano-2-(methylsulfonyl)benzamide

To a solution of 1-(6-amino-2,4-difluoro-3-(2-(tetrahydro-2H-pyran-4-yl)ethyl)phenyl)ethan-1-one (170.0 mg, 175.2 mmol) in isopropyl acetate (3 mL) was added 5-cyano-2-(methylsulfonyl)benzoyl chloride (175.2 mg, 0.72 mmol) in portion at r.t. The mixture was degassed and purged with N₂ for 2 min. The resulting mixture was sealed and stirred at 80° C. for 18 h. The mixture was quenched with water (10 mL) and extracted with EA (30 mL×3). The combined organic layers were washed with brine, dried over Na₂SO₄ and filtered. The mixture was concentrated under reduced pressure to give a crude product which was purified by flash chromatography on a silica gel column eluting with PE/EA (3/1) to afford the title compound (200 mg, 56.6% yield) as a white solid. LCMS calc. for C₂₄H₂₅F₂N₂O₅S [M+H]⁺: m/z=491.3; Found: 491.3.

### Step 4: 3-(5,7-difluoro-4-oxo-6-(2-(tetrahydro-2H-pyran-4-yl)ethyl)-1,4-dihydroquinolin-2-yl)-4-(methylsulfonyl)benzonitrile

To a solution of N-(2-acetyl-3,5-difluoro-4-(2-(tetrahydro-2H-pyran-4-yl)ethyl)phenyl)-5-cyano-2-(methylsulfonyl)benzamide (200.0 mg, 0.41 mmol) in 2-methyltetrahydrofuran (5 mL) was added LiOtBu (52.0 mg, 0.65 mmol) at 0° C. under N₂. The mixture was stirred at 75° C. overnight under N₂. The reaction mixture was quenched with aq. HCl (0.7 mL, 2 M) and concentrated under reduced pressure. The residue was purified by flash chromatography on a silica gel column eluting with DCM/MeOH (40/1) to afford the title compound (12.3 mg, 6.4% yield) as a white solid. ¹H NMR (400 MHz, DMSO-d₆) δ 12.06 (s, 1H), 8.38-8.27 (m, 3H), 7.06 (d, J=10.4 Hz, 11H), 6.10 (d, J=1.4 Hz, 1H), 3.89-3.81 (m, 2H), 3.31-3.23 (m, 51H), 2.72-2.66 (m, 2H), 1.71-1.62 (m, 2H), 1.55-1.43 (m, 31H), 1.25-1.11 (m, 2H). LCMS calc. for C₂₄H₂₃F₂N₂O₄S [M+H]⁺: m/z=473.5; Found: 473.5.

### Example 26: 3-(6-(Cyclobutylmethoxy)-5,7-difluoro-4-oxo-1,4-dihydroquinolin-2-yl)-4-(methylsulfonyl)benzonitrile

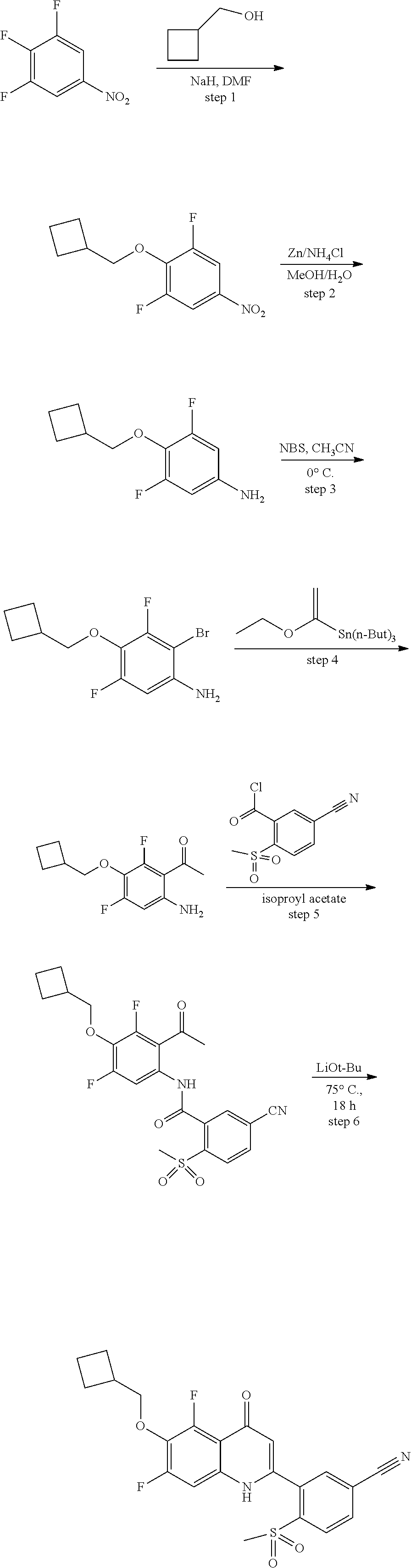

### Step 1: 2-(cyclobutylmethoxy)-1,3-difluoro-5-nitrobenzene

To a mixture of 1,2,3-trifluoro-5-nitrobenzene (1.0 g, 5.65 mmol) and cyclobutylmethanol (584.0 mg, 6.78 mmol) in DMF (12 mL) was added NaH (60 wt. %, 452 mg) in portion at 0° C. under N₂. The reaction mixture was stirred at r.t. for 18 h. under N₂. The reaction mixture was quenched with cold sat. NaHCO₃ aqueous (10 mL). The mixture was extracted with EA (100 mL×3). The combined organic layers were dried over with Na₂SO₄, filtered and concentrated under reduced pressure. The residue was purified by flash chromatography on a silica gel column eluting with PE/EA (100/3) to afford the title compound (1.26 g, 76.4% yield) as a white solid. ¹H NMR (400 MHz, DMSO-d₆) δ 8.23-8.08 (m, 21H), 4.29 (d, J=6.7 Hz, 2H), 2.79-2.63 (m, 1H), 2.08-1.99 (m, 2H), 1.96-1.76 (in, 4H).

### Step 2: 4-(cyclobutylmethoxy)-3,5-difluoroaniline

To a mixture of 2-(cyclobutylmethoxy)-1,3-difluoro-5-nitrobenzene (1.26 g, 5.1 mmol) in MeOH (10 mL) and sat. NH₄Cl aqueous (4 mL) was added Zn powder (3.3 g, 51.0 mmol) at r.t. The mixture was stirred at r.t. for 2 h. The reaction mixture was quenched with water (25 mL) and extracted with EA (30 mL×3). The combined organic layers were washed with brine, dried over Na₂SO₄, filtered and concentrated under reduced pressure. The residue was purified by flash chromatography on a silica gel column eluting with PE/EA (15/1) to afford the title compound (960 mg, 88.3% yield) as a yellow oil. ¹H NMR (400 MHz, DMSO-d₆) δ 6.31-6.11 (m, 2H), 5.37 (s, 2H), 3.83 (d, J=6.7 Hz, 2H), 2.60 (dt, J=14.6, 7.4 Hz, 1H), 2.06-1.95 (m, 2H), 1.91-1.70 (m, 4H). LCMS calc. for C₁₁H₁₄F₂NO [M+H]⁺: m/z=214.3; Found: 214.3.

### Step 3: 2-bromo-4-(cyclobutylmethoxy)-3,5-difluoroaniline

To a solution of 4-(cyclobutylmethoxy)-3,5-difluoroaniline (500.0 mg, 2.34 mmol) in CH₃CN (8 mL) was added NBS (396.9 mg, 2.34 mmol) in portion at 0° C. The resulting mixture was stirred at 0° C. for 20 min. The mixture was quenched with water (10 mL) and extracted with EA (30 mL×3). The combined organic layers were washed with NaHCO₃ aqueous, brine, dried over Na₂SO₄ and filtered. The filtrate was concentrated under reduced pressure. The residue was purified by flash chromatography on a silica gel column eluting with PE/EA (12/1) to afford the title compound (550 mg, 81.0% yield) as a red oil. LCMS calc. for C₁₁H₁₃BrF₂NO [M+H]⁺: m/z=292.1; Found: 292.1.

### Step 4: 1-(6-amino-3-(cyclobutylmethoxy)-2,4-difluorophenyl)ethan-1-one

A mixture of 2-bromo-4-(cyclobutylmethoxy)-3,5-difluoroaniline (300.0 mg, 1.03 mmol), tributyl(1-ethoxyvinyl)stannane (558.0 mg, 1.55 mmol) and Pd(PPh₃)₄ (118.9 mg, 0.10 mmol) in 1,4-dioxane (3 mL) was charged with N₂ and stirred at 100° C. for 18 h. The mixture was concentrated under reduced pressure. The residue was dissolved in acetone (3 mL) and HCl (1.5 mL, 2 N). The mixture was stirred at r.t. for 2 h. and adjusted to PH-7-8 with sat. NaHCO₃ (aq.) at 0° C. The mixture was extracted with EA (15 mL×3). The combined organic layers were dried over with Na₂SO₄, filtered, and concentrated under reduced pressure. The result was purified by flash chromatography on a silica gel column eluting with PE/EA (100/7) to afford the title compound (100.0 mg, 38.1% yield) as a yellow oil. LCMS calc. for C₁₃H₁₆F₂NO₂ [M+H]⁺: m/z=256.2; Found: 256.2.

### Step 5: N-(2-acetyl-4-(cyclobutylmethoxy)-3,5-difluorophenyl)-5-cyano-2-(methylsulfonyl)benzamide

To a solution of 1-(6-amino-3-(cyclobutylmethoxy)-2,4-difluorophenyl)ethan-1-one (75.0 mg, 0.29 mmol) in isopropyl acetate (1.5 mL) was added 5-cyano-2-(methylsulfonyl)benzoyl chloride (85.7 mg, 0.35 mmol) in portion at r.t. The mixture was degassed and purged with N₂ for 2 min. The resulting mixture was stirred at 80° C. for 18 h. The mixture was quenched with water (10 mL) and extracted with EA (30 mL×3). The combined organic layers were washed with brine, dried over Na₂SO₄, filtered and concentrated under reduced pressure. The residue was purified by flash chromatography on a silica gel column eluting with PE/EA (5/1) to afford the title compound (70 mg, 52.2% yield) as a white solid. LCMS calc. for C₂₂H₂₁F₂N₂O₅S [M+H]⁺: m/z=463.2; Found: 463.2.

### Step 6: 3-(6-(cyclobutylmethoxy)-5,7-difluoro-4-oxo-1,4-dihydroquinolin-2-yl)-4-(methylsulfonyl)benzonitrile

A mixture of N-(2-acetyl-4-(cyclobutylmethoxy)-3,5-difluorophenyl)-5-cyano-2-(methylsulfonyl)benzamide (55.0 mg, 0.12 mmol) and LiOtBu (15.2 mg, 0.19 mmol) in 2-methyltetrahydrofuran (2 mL) was stirred at 75° C. overnight. The reaction mixture was quenched with aq. HCl (0.3 mL, 2 M) and concentrated under reduced pressure. The residue was purified by flash chromatography on a silica gel column eluting with DCM/MeOH (40/1) to afford the title compound (3.6 mg, 6.7% yield) as a white solid. ¹H NMR (400 MHz, DMSO-d₆) δ 12.09 (s, 1H), 8.41-8.26 (m, 3H), 7.15 (d, J=11.1 Hz, 11H), 6.08 (s, 1H), 4.07 (d, J=6.6 Hz, 2H), 3.29 (s, 3H), 2.12-1.98 (m, 3H), 1.92-1.79 (m, 4H). LCMS calc. for C₂₂H₂₀F₂N₂O₄S [M+H]⁺: m/z=445.1; Found: 445.4.

### Example 27: 3-(5,7-Difluoro-6-((2-methylpyridin-4-yl)ethynyl)-4-oxo-1,4-dihydroquinolin-2-yl)-4-(methylsulfonyl)benzonitrile

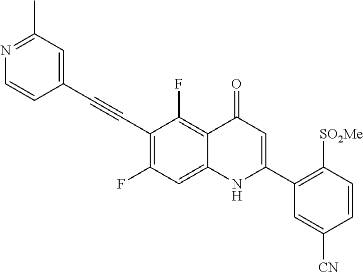 [SMILES: Cc1cc(C#Cc2c(F)cc3[nH]c(-c4cc(C#N)ccc4S(C)(=O)=O)cc(=O)c3c2F)ccn1]

A mixture of 3-(5,7-difluoro-6-iodo-4-oxo-1,4-dihydroquinolin-2-yl)-4-(methylsulfonyl)benzonitrile (50 mg, 0.103 mmol, Example 1 Step 5), 4-ethynyl-2-methylpyridine (18.0 mg, 0.154 mmol), CuI (2 mg, 0.0103 mmol) and PdCl₂(PPh₃)₂ (7.23 mg, 0.0103 mmol) in TEA (0.3 mL) and DMF (1 mL) was degassed and recharged with nitrogen for 3 cycles, and then stirred at 60° C. for 2 h. The reaction mixture was diluted with water (10 mL) and extracted with FA (10 mL×2). The combined organic layers were washed with brine, dried over Na₂SO₄, filtered and concentrated under reduced pressure. The residue was purified by prep-HPLC (Instrument: GILSON-281 Column: Xridge C18 250*19.00 mml 10 um Temperature: 25° C. Mobile phase: A: H₂O (10 mM NH₄HCO₃) B: ACN Flow rate: 20.0 ml/mi Gradient: 30-60 Detection wavelength: 214/254 nm Retention time: 6-14 min) to afford the title compound (3.61 mg, 7.4% yield) as a white solid. ¹H NMR (400 MHz, DMSO-d₆) 12.39 (s, 1H), 8.54 (d, J=5.0 Hz, 1H), 8.43-8.17 (m, 3H), 7.47 (s, 1H), 7.37 (d, J=5.0 Hz, 1H), 7.23 (s, 1H), 6.24 (s, 1H), 3.30 (s, 3H), 2.52 (s, 3H). LCMS calc. for C₂₅H₁₆F₂N₃O₃S [M+H]⁺: m/z=476.1; Found: 476.4.

### Example 28: 3-(5,7-Difluoro-6-((3-methylpyridin-4-yl)ethynyl)-4-oxo-1,4-dihydroquinolin-2-yl)-4-(methylsulfonyl)benzonitrile

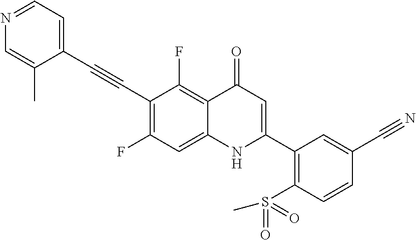 [SMILES: Cc1cnccc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (50 mg, 0.1 mmol, Example 1 Step 5), 4-ethynyl-3-methylpyridine (18.1 mg, 0.15 mmol), CuI (1.9 mg, 0.01 mmol) and PdCl₂(PPh₃)₂ (7.0 mg, 0.01 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (12.2 mg, 25.6% yield) as a yellow solid. ¹H NMR (400 MHz, DMSO-d₆) δ 12.38 (s, 1H), 8.61 (s, 1H), 8.49 (d, J=5.0 Hz, 1H), 8.41-8.22 (m, 3H), 7.51 (d, J=5.0 Hz, 1H), 7.23 (s, 1H), 6.24 (s, 1H), 3.34 (s, 3H), 2.47 (s, 3H). LCMS calc. for C₂₅H₁₆F₂N₃O₃S [M+H]⁺: m/z=: 476.4; Found: 476.4.

### Example 29: 3-(5,7-Difluoro-6-((2-methylpyridin-3-yl)ethynyl)-4-oxo-1,4-dihydroquinolin-2-yl)-4-(methylsulfonyl)benzonitrile

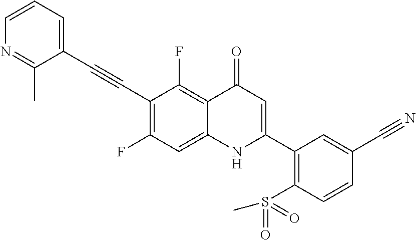 [SMILES: Cc1ncccc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (65 mg, 0.134 mmol, Example 1 Step 5), 3-ethynyl-2-methylpyridine (24 tug, 0.2 mmol), Pd(PPh₃)₂Cl₂ (10 tug, 0.013 mmol), CuI (3 tug, 0.013 mmol) and TEA (0.5 mL) in DMF (1 mL) was degassed and charged with N₂ and then stirred at 75° C. for 8 h. The reaction mixture was diluted with water (20 mL) and extracted with EA (10 mL×3). The combined organic layers were washed with brine, dried over anhydrous Na₂SO₄, filtered and concentrated under reduced pressure. The residue was purified by flash chromatography on a silica gel column eluting with MeOH/DCM (2%) to afford the crude product, which was washed with MeOH and dried in vacuum to afford the title compound (25.1 tug, 40% yield) as a yellow solid. ¹H NMR (400 MHz, DMSO-d₆) (12.34 (s, 1H), 8.52 (dd, J=4.8, 1.3 Hz, 1H), 8.41-8.21 (m, 3H), 7.95 (d, J=7.5 Hz, 111), 7.33 (dd, J=7.7, 4.9 Hz, 1H), 7.21 (d, J=9.1 Hz, 1H), 6.22 (s, 1H), 3.32 (s, 3H), 2.70 (s, 3H). LCMS calc. for C₂₅H₁₆F₂N₃O₃S [M+H]⁺: m/z=476.0; Found: 476.1.

### Example 30: 3-(5,7-Difluoro-6-((2-methoxyphenyl)ethynyl)-4-oxo-1,4-dihydroquinolin-2-yl)-4-(methylsulfonyl)benzonitrile

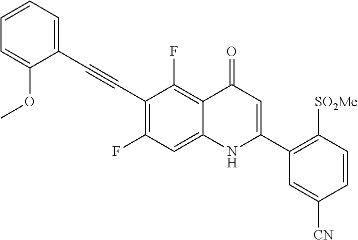 [SMILES: COc1ccccc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (50 mg, 0.103 mmol, Example 1 Step 5), 1-ethynyl-2-methoxybenzene (20.3 mg, 0.154 mmol), Cut (2 tug, 0.0103 mmol) and PdCl₂(PPh₃)₂ (7.23 mg, 0.0103 mmol) in TEA (0.3 mL) and DMF (1 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (13.32 mg, 26.4% yield) as a white solid. ¹H NMR (400 MHz, DMSO-d₆) δ 12.31 (s, 1H), 8.43-8.21 (m, 3H), 7.56-7.39 (m, 2H), 7.23-7.09 (m, 2H), 7.02 (t, J=7.3 Hz, 1H), 6.20 (s, 1H), 3.89 (s, 3H). LCMS calc. for C₂₆H₁₇F₂N₂O₄S [M+H]⁺: m/z=491.4; Found: 491.4.

### Example 31: 3-(5,7-Difluoro-6-((6-methylpyridin-2-yl)ethynyl)-4-oxo-1,4-dihydroquinolin-2-yl)-4-(methylsulfonyl)benzonitrile

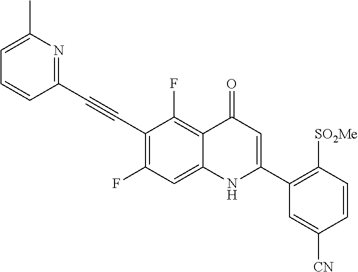 [SMILES: Cc1cccc(C#Cc2c(F)cc3[nH]c(-c4cc(C#N)ccc4S(C)(=O)=O)cc(=O)c3c2F)n1]

A mixture of 3-(5,7-difluoro-6-iodo-4-oxo-1,4-dihydroquinolin-2-yl)-4-(methylsulfonyl)benzonitrile (50 mg, 0.103 mmol, Example 1 Step 5), 2-ethynyl-6-methylpyridine (18.0 mg, 0.154 mmol), CuI (2 mg, 0.0103 mmol) and PdCl₂(PPh₃)₂ (7.23 mg, 0.0103 mmol) in TEA (0.3 mL) and DMF (1 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (22.1 mg, 45.1% yield) as a yellow solid. ¹H NMR (400 MHz, DMSO-d₆) δ 12.38 (s, 1H), 8.49-8.20 (m, 3H), 7.79 (t, J=7.8 Hz, 1H), 7.51 (d, J=7.6 Hz, 1H), 7.35 (d, J=7.8 Hz, 1H), 7.26 (s, 1H), 6.27 (s, 1H), 3.38 (s, 3H), 2.55 (s, 3H). LCMS calc. for C₂₅H₁₆F₂N₃O₃S [M+H]⁺: m/z=476.3; Found: 476.3.

### Example 32: 3-(6-((3,5-Dimethylpyridin-4-yl)ethynyl)-5,7-difluoro-4-oxo-1,4-dihydroquinolin-2-yl)-4-(methylsulfonyl)benzonitrile

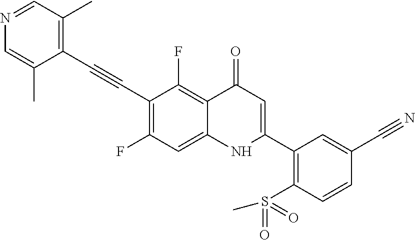 [SMILES: Cc1cncc(C)c1C#Cc1c(F)cc2[nH]c(-c3cc(C#N)ccc3S(C)(=O)=O)cc(=O)c2c1F]

To a solution of 3-(5,7-difluoro-6-iodo-4-oxo-1,4-dihydroquinolin-2-yl)-4-(methylsulfonyl)benzonitrile (65 mg, 0.13 mmol, Example 1 Step 5) and 4-ethynyl-3,5-dimethylpyridine (44 mg, 0.33 mmol) in N,N-dimethylformamide (1.5 mL) were added Pd(PPh₃)₂Cl₂ (9.1 mg, 0.013 mmol), CuI (2.5 mg, 0.013 mmol) and triethylamine (0.6 mL) under N₂ atmosphere. The mixture was stirred at 75° C. overnight. The mixture was diluted with water (10 mL) and extracted with EA (10 mL×3). The combined organic layers were washed with brine, dried over Na₂SO₄, filtered, and concentrated under reduced pressure. The residue was purified by flash chromatography on a silica gel column eluting with MeOH/DCM (1/40) to afford the title compound (11.2 mg, 17.2% yield) as a yellow solid. ¹H NMR (401 MHz, DMSO) δ 12.38 (s, 1H), 8.42 (s, 2H), 8.32 (m, 3H), 7.24 (s, 1H), 6.24 (s, 1H), 3.33 (s, 3H), 2.46 (s, 6H). LCMS calc. for C₂₆H₁₈F₂N₃O₃S [M+H]⁺: m/z=490.5; Found: 490.0.

### Example 33: 3-(6-((2-Chlorophenyl)ethynyl)-5,7-difluoro-4-oxo-1,4-dihydroquinolin-2-yl)-4-(methylsulfonyl)benzonitrile

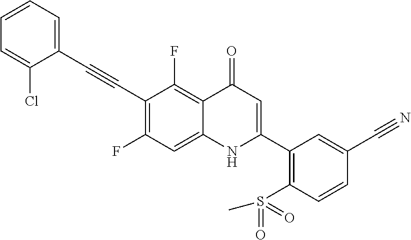 [SMILES: CS(=O)(=O)c1ccc(C#N)cc1-c1cc(=O)c2c(F)c(C#Cc3ccccc3Cl)c(F)cc2[nH]1]

A mixture of 3-(5,7-difluoro-6-iodo-4-oxo-1,4-dihydroquinolin-2-yl)-4-(methylsulfonyl)benzonitrile (55 mg, 0.113 mmol, Example 1 Step 5), 1-chloro-2-ethynylbenzene (31 mg, 0.226 mmol), Pd(PPh₃)₂Cl₂ (8 mg, 0.011 mmol), Cut (3 mg, 0.011 mmol) and TEA (0.5 mL) in DMF (1 mL) was degassed and charged with N₂ and then stirred at 65° C. for 5 h. The reaction mixture was diluted with water (20 mL) and extracted with EA (10 mL×3). The combined organic layers were washed with brine, dried over anhydrous Na₂SO₄, filtered and concentrated under reduced pressure. The residue was purified by flash chromatography on a silica gel column eluting with MeOH/DCM (2%) to afford crude product, which was washed with MeOH and dried in vacuum to afford the title compound (18.7 mg, 33% yield) as a yellow solid. ¹H NMR (400 MHz, DMSO-d₆) δ 12.35 (s, 1H), 8.42-8.20 (m, 3H), 7.73 (dd, J=7.5, 1.5 Hz, 1H), 7.65 (dd, J=8.0, 0.9 Hz, 1H), 7.51 (td, J=7.8, 1.8 Hz, 1H), 7.45 (td, J=7.5, 1.2 Hz, 1H), 7.23 (s, 1H), 6.24 (s, 1H), 3.33 (s, 3H). LCMS calc. for C₂₅H₁₄ClF₂N₂O₃S [M+H]⁺: m/z=495.0; Found: 495.0.

### Example 34: 3-(5,7-Difluoro-4-oxo-6-((tetrahydrofuran-3-yl)ethynyl)-1,4-dihydroquinolin-2-yl)-4-(methylsulfonyl)benzonitrile

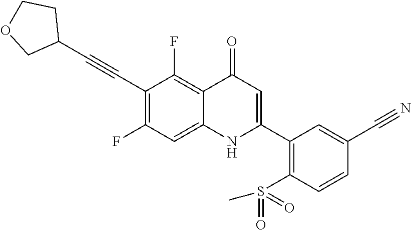 [SMILES: CS(=O)(=O)c1ccc(C#N)cc1-c1cc(=O)c2c(F)c(C#CC3CCOC3)c(F)cc2[nH]1]

A mixture of 3-(5,7-difluoro-6-iodo-4-oxo-1,4-dihydroquinolin-2-yl)-4-(methylsulfonyl)benzonitrile (100 mg, 0.206 mmol, Example 1 Step 5), 3-ethynyltetrahydrofuran (29.6 mg, 0.308 mmol), Cut (4.0 mg, 0.020 mmol) and PdCl₂(PPh₃)₂ (14.46 mg, 0.02 mmol) in TEA (IL) and DMF (3 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 nM NH₄HCO₃) B: ACN Flow rate: 20.0 ml/min Gradient: 30-60 Detection wavelength: 214/254 nm Retention time: 6-14 min) to afford the title compound (45 mg, 48.1% yield) as a yellow solid. ¹H NMR (400 MHz, DMSO-d₆) δ 12.25 (s, 1H), 8.49-8.19 (m, 3H), 7.16 (s, 1H), 6.18 (s, 1H), 4.01 (t, J=7.7 Hz, 1H), 3.91-3.83 (m, 1H), 3.77 (dd, J=14.4, 7.8 Hz, 1H), 3.64 (dd, J=8.0, 6.5 Hz, 1H), 3.47-3.33 (m, 4H), 2.34-2.25 (m, 1H), 2.05-1.89 (m, 1H). LCMS calc. for C₂₃H₁₇F₂N₂O₄S [M+H]+: m/z=455.5; Found: 455.5.

### Example 35: 3-(6-((3,5-Dimethylisoxazol-4-yl)ethynyl)-5,7-difluoro-4-oxo-1,4-dihydroquinolin-2-yl)-4-(methylsulfonyl)benzonitrile

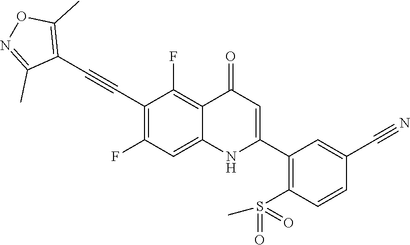 [SMILES: Cc1noc(C)c1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 4-ethynyl-3,5-dimethylisoxazole (23 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 70° C. for 5 h. The reaction mixture was diluted with water (10 mL) and extracted with EA (10 mL×2). The combined organic layers were washed with brine, dried over Na₂SO₄, filtered and concentrated tinder reduced pressure. The residue was purified by flash chromatography on a silica gel column eluting with MeOH/DCM (2%) to afford crude product which was washed with MeOH and dried in vacuum to afford the title compound (16.3 mg, 28% yield) as a yellow solid. ¹H NMR (400 MHz, DMSO-d₆) δ 12.33 (s, 1H), 8.41-8.23 (m, 3H), 7.20 (s, 1H), 6.21 (s, 1H), 3.33 (s, 3H), 2.54 (s, 3H), 2.32 (s, 3H). LCMS calc. for C₂₄H₁₆F₂N₃O₄S [M+H]⁺: m/z=480.1; Found: 480.1.

### Example 36: 3-(5,7-Difluoro-4-oxo-6-((2-(trifluoromethoxy)phenyl)ethynyl)-1,4-dihydroquinolin-2-yl)-4-(methylsulfonyl)benzonitrile

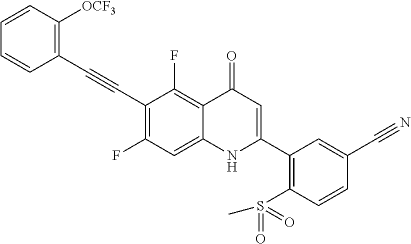 [SMILES: CS(=O)(=O)c1ccc(C#N)cc1-c1cc(=O)c2c(F)c(C#Cc3ccccc3OC(F)(F)F)c(F)cc2[nH]1]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 1-ethynyl-2-(trifluoromethoxy)benzene (35 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 65° C. for 3 h. The reaction mixture was diluted with water (10 mL) and extracted with EA (10 mL×2). The combined organic layers were washed with brine, dried over Na₂SO₄, filtered and concentrated under reduced pressure. The residue was purified by flash chromatography on a silica gel column eluting with MeOH/DCM (2%) to afford crude product which was washed with MeOH and dried in vacuum to afford the title compound (9.9 mg, 15% yield) as a yellow solid. ¹H NMR (400 MHz, DMSO-d₆) δ 12.37 (s, 1H), 8.42-8.25 (m, 3H), 7.78 (dd, J=7.6, 1.4 Hz, 1H), 7.66-7.60 (m, 1H), 7.56 (d, J=8.3 Hz, 1H), 7.54-7.50 (m, 1H), 7.26 (s, 1H), 6.26 (s, 1H), 3.36 (s, 3H). LCMS calc. for C₂₆H₁₄F₅N₂O₄S [M+H]⁺: m/z=545.0; Found: 545.1.

### Example 37: 3-(6-((2-(Difluoromethoxy)phenyl)ethynyl)-5,7-difluoro-4-oxo-1,4-dihydroquinolin-2-yl)-4-(methylsulfonyl)benzonitrile

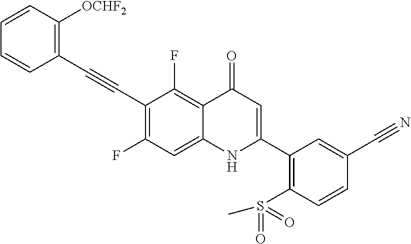 [SMILES: CS(=O)(=O)c1ccc(C#N)cc1-c1cc(=O)c2c(F)c(C#Cc3ccccc3OC(F)F)c(F)cc2[nH]1]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 1-(difluoromethoxy)-2-ethynylbenzene (31 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 65° C. for 3 h. The reaction mixture was diluted with water (10 mL) and extracted with EA (10 mL×2). The combined organic layers were washed with brine, dried over Na₂SO₄, filtered and concentrated under reduced pressure. The residue was purified by flash chromatography on a silica gel column eluting with MeOH/DCM (2%) to afford crude product which was washed with MeOH and dried in vacuum to afford the title compound (9.3 mg, 14% yield) as a yellow solid. ¹H NMR (400 MHz, DMSO-d₆) δ 12.34 (s, 1H), 8.47-8.20 (m, 3H), 7.70 (dd, J=7.8, 1.2 Hz, 1H), 7.58-7.53 (m, 1H), 7.53-7.31 (m, 3H), 7.26-7.12 (m, 1H), 6.21 (s, 1H), 3.33 (s, 3H). LCMS calc. for C₂₆H₁₅F₄N₂O₄S [M+H]⁺: m/z=527.0; Found: 527.0.

### Example 38: 3-(5,7-Difluoro-6-((3-methoxypyridin-2-yl)ethynyl)-4-oxo-1,4-dihydroquinolin-2-yl)-4-(methylsulfonyl)benzonitrile

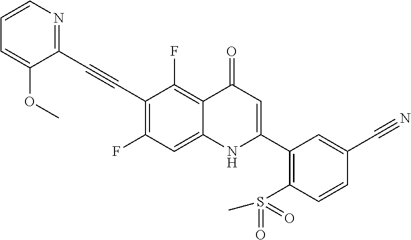 [SMILES: COc1cccnc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 4-ethynyl-3-methoxypyridine (25 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 70° C. for 5 h. The reaction mixture was diluted with water (10 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford crude product which was further purified by prep-HPLC (MeCN/water with 0.1% NH₃·H₂O=0/100~100/0) to afford the title compound (12.4 mg, 21% yield) as a yellow solid. ¹H NMR (400 MHz, DMSO-d₆) 312.34 (s, 1H), 8.53 (s, 1H), 8.39-8.29 (m, 3H), 8.28 (d, J=4.8 Hz, 1H), 7.51 (d, J=4.8 Hz, 1H), 7.26 (s, 1H), 6.28 (s, 1H), 4.01 (s, 3H), 3.35 (s, 3H). LCMS calc. for C₂₅H₁₆F₂N₃O₄S [M+Hf]: m/z=492.1; Found: 492.1.

### Example 39: 3-(5,7-Difluoro-4-oxo-6-(p-tolylethynyl)-1,4-dihydroquinolin-2-yl)-4-(methylsulfonyl)benzonitrile

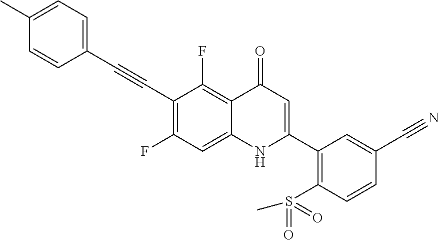 [SMILES: Cc1ccc(C#Cc2c(F)cc3[nH]c(-c4cc(C#N)ccc4S(C)(=O)=O)cc(=O)c3c2F)cc1]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 1-ethynyl-4-methylbenzene (22 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 65° C. for 3 h. The reaction mixture was diluted with water (10 mL) and extracted with FA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford crude product which was washed with MeOH and dried in vacuum to afford the title compound (23 tug, 39% yield) as a yellow solid. ¹H NMR (400 MHz, DMSO-d₆) δ 12.30 (s, 1H), 8.41-8.19 (m, 3H), 7.50 (d, J=7.8 Hz, 2H), 7.29 (d, J=7.9 Hz, 2H), 7.18 (d, J=9.7 Hz, 1H), 6.20 (s, 1H), 3.31 (s, 3H), 2.37 (s, 3H). LCMS calc. for C₂₆H₁₇F₂N₂O₃S [M+H]⁺: m/z=475.1; Found: 475.1.

### Example 40: 3-(5,7-Difluoro-6-(imidazo[1,2-h]pyridazin-3-ylethynyl)-4-oxo-1,4-dihydroquinolin-2-yl)-4-(methylsulfonyl)benzonitrile

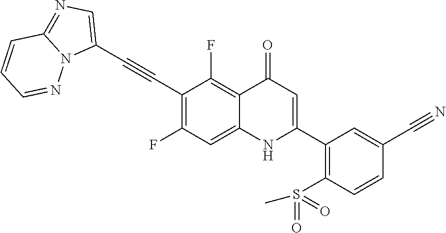 [SMILES: CS(=O)(=O)c1ccc(C#N)cc1-c1cc(=O)c2c(F)c(C#Cc3cnc4cccnn34)c(F)cc2[nH]1]

A mixture of 3-(5,7-difluoro-6-iodo-4-oxo-1,4-dihydroquinolin-2-yl)-4-(methylsulfonyl)benzonitrile (60 tug, 0,123 mmol, Example 1 Step 5), 3-ethynylimidazo[1,2-b]pyridazine (27 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 75° C. for 16 h. The reaction mixture was diluted with water (10 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford crude product which was further purified by prep-HPLC (MeCN/water with 0.1% NH₃·H₂O=0/100~100/0) to afford the title compound (7.2 mg, 12% yield) as a yellow solid. ¹H NMR (400 MHz, DMSO-d₆) δ 12.36 (s, 1H), 8.74 (d, J=3.6 Hz, 1H), 8.41-8.24 (m, 5H), 7.43 (dd, J=9.2, 4.4 Hz, 1H), 7.22 (d, J=9.4 Hz, 1H), 6.22 (s, 1H), 3.32 (s, 31H). LCMS calc. for C₂₅H₁₄F₂N₅O₃S [M+H]⁺: m/z=502.1; Found: 502.1.

### Example 41: 3-(5,7-Difluoro-6-((3-methoxypyridin-2-yl)ethynyl)-4-oxo-1,4-dihydroquinolin-2-yl)-4-(methylsulfonyl)benzonitrile

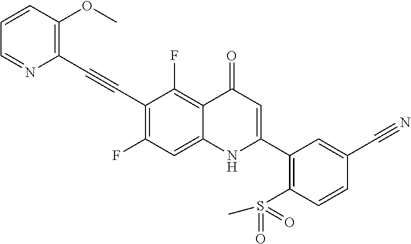 [SMILES: COc1cccnc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (60 mg, 0.123 mmol, Example 1 Step 5), 2-ethynyl-3-methoxypyridine (25 mg, 0.185 mmol), CuI (3 mg, 0.012 mmol) and PdCl₂(PPh₃)₂ (9 mg, 0.012 mmol) in TEA (0.4 mL) and DMF (1 mL) was degassed and recharged with nitrogen for 3 cycles, and then stirred at 75° C. for 5 h. The reaction mixture was diluted with water (10 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford crude product which was washed with MeOH and dried in vacuum to afford the title compound (10.9 mg, 18% yield) as a white solid. ¹H NMR (400 MHz, DMSO-d₆) δ 12.34 (s, 1H), 8.41-8.25 (m, 3H), 8.22 (d, J=4.1 Hz, 1H), 7.61 (d, J=8.4 Hz, 1H), 7.47 (dd, J=8.5, 4.6 Hz, 1H), 7.20 (d, J=9.8 Hz, 1H), 6.22 (s, 1H), 3.93 (s, 3H), 3.32 (s, 3H). LCMS calc. for C₂₅H₁₆F₂N₃O₄S [M±H]⁺: m/z=492.1; Found: 492.0.

### Example 42: 3-(6-((2-Cyanophenyl)ethynyl)-5,7-difluoro-4-oxo-1,4-dihydroquinolin-2-yl)-4-(methylsulfonyl)benzonitrile

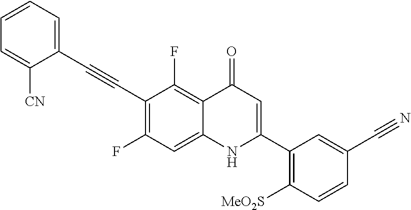 [SMILES: CS(=O)(=O)c1ccc(C#N)cc1-c1cc(=O)c2c(F)c(C#Cc3ccccc3C#N)c(F)cc2[nH]1]

A mixture of 3-(5,7-difluoro-6-iodo-4-oxo-1,4-dihydroquinolin-2-yl)-4-(methylsulfonyl)benzonitrile (50 mg, 0.1 mmol, Example 1 Step 5), 2-ethynylbenzonitrile (19.6 mg, 0.15 mmol), CuI (1.9 mg, 0.01 mmol) and PdCl₂(PPh₃)₂ (7.0 mg, 0.01 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (23.4 mg, 48.2% yield) as a yellow solid. ¹H NMR (400 MHz, DMSO-d₆) δ 12.40 (s, 1H), 8.38-8.25 (m, 3H), 8.04-7.97 (m, 1H), 7.88-7.77 (m, 2H), 7.67 (td, J=7.6, 1.5 Hz, 1H), 7.29 (s, 1H), 6.31 (s, 1H), 3.37 (s, 3H). LCMS calc. for C₂₆H₁₄F₂N₃O₃S [M+H]⁺: m/z=486.4; Found: 486.4.

### Example 43: 3-(5,7-Difluoro-6-((3-methoxypyrazin-2-yl)ethynyl)-4-oxo-1,4-dihydroquinolin-2-yl)-4-(methylsulfonyl)benzonitrile

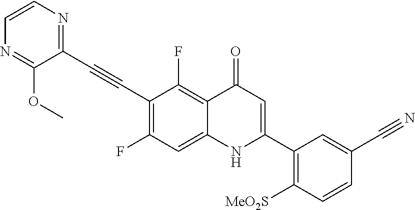 [SMILES: COc1nccnc1C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (40 mg, 0.08 mmol, Example 1 Step 5), 2-ethynyl-3-methoxypyrazine (16.5 mg, 0.12 mmol), CuI (1.5 mg, 0.008 mmol) and PdCl₂(PPh₃)₂ (5.6 mg, 0.008 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 mi/min Gradient: 30-60 Detection wavelength: 214/254 nm Retention time: 6-14 min) to afford the title compound (19.2 mg, 48.8% yield) as a yellow solid. ¹H NMR (400 MHz, DMSO-d₆) δ 12.40 (s, 1H), 8.50-8.07 (m, 5H), 7.25 (s, 1H), 6.30 (s, 1H), 4.03 (s, 3H), 3.38 (s, 3H). LCMS calc. for C₂₄H₁₅F₂N₄O₄S [M+H]⁺: m/z=493.4; Found: 493.4.

### Example 44: 3-(5,7-Difluoro-4-oxo-6-((2-(trifluoromethyl)phenyl)ethynyl)-1,4-dihydroquinolin-2-yl)-4-(methylsulfonyl)benzonitrile

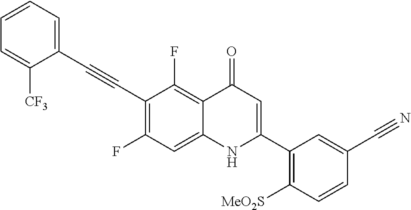 [SMILES: CS(=O)(=O)c1ccc(C#N)cc1-c1cc(=O)c2c(F)c(C#Cc3ccccc3C(F)(F)F)c(F)cc2[nH]1]

A mixture of 3-(5,7-difluoro-6-iodo-4-oxo-1,4-dihydroquinolin-2-yl)-4-(methylsulfonyl)benzonitrile (50 mg, 0.1 mmol, Example 1 Step 5), 1-ethynyl-2-(trifluoromethyl)benzene (26.2 mg, 0.15 mmol), CuI (1.9 mg, 0.01 mmol) and PdCl₂(PPh₃)₂ (7.0 mg, 0.01 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mm 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (6.2 mg, 11.7% yield) as a yellow solid. ¹H NMR (400 MHz, DMSO-d₆) δ 12.38 (s, 1H), 8.41-8.24 (m, 31H), 7.93-7.84 (m, 2H), 7.78 (t, J=7.5 Hz, 1H), 7.75-7.64 (m, 1H), 7.22 (s, 11H), 6.24 (s, 1H), 3.31-3.25 (m, 3H). LCMS calc. for C₂₆H₁₄F₅N₂O₃S [M+H]⁺: m/z=529.4; Found: 529.4.

### Example 45: 3-(5,7-Difluoro-6-((3-methyloxetan-3-yl)ethynyl)-4-oxo-1,4-dihydroquinolin-2-yl)-4-(methylsulfonyl)benzonitrile

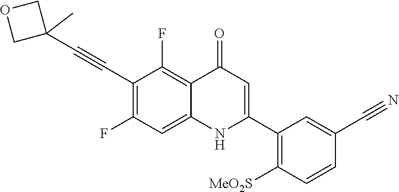 [SMILES: CC1(C#Cc2c(F)cc3[nH]c(-c4cc(C#N)ccc4S(C)(=O)=O)cc(=O)c3c2F)COC1]

A mixture of 3-(5,7-difluoro-6-iodo-4-oxo-1,4-dihydroquinolin-2-yl)-4-(methylsulfonyl)benzonitrile (40 mg, 0.08 mmol, Example 1 Step 5), 3-ethynyl-3-methyloxetane (11.5 mg, 0.12 mmol), CuI (1.5 mg, 0.008 mmol) and PdCl₂(PPh₃)₂ (5.6 mg, 0.008 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (18.1 mg, 49.8% yield) as a white solid. ¹H NMR (400 MHz, DMSO-d₆) δ 12.27 (s, 11H), 8.47-8.17 (m, 3H), 7.15 (s, 1H), 6.18 (s, 1H), 4.77 (d, J=5.6 Hz, 2H), 4.49 (d, J=5.6 Hz, 2H), 3.30-3.23 (m, 3H), 1.68 (s, 3H). LCMS calc. for C₂₃H₁₇F₂N₂O₄S [M+H]⁺: m/z=455.4; Found: 455.4.

### Example 46: 3-(5,7-Difluoro-6-(oxetan-3-ylethynyl)-4-oxo-1,4-dihydroquinolin-2-yl)-4-(ethylsulfonyl)benzonitrile

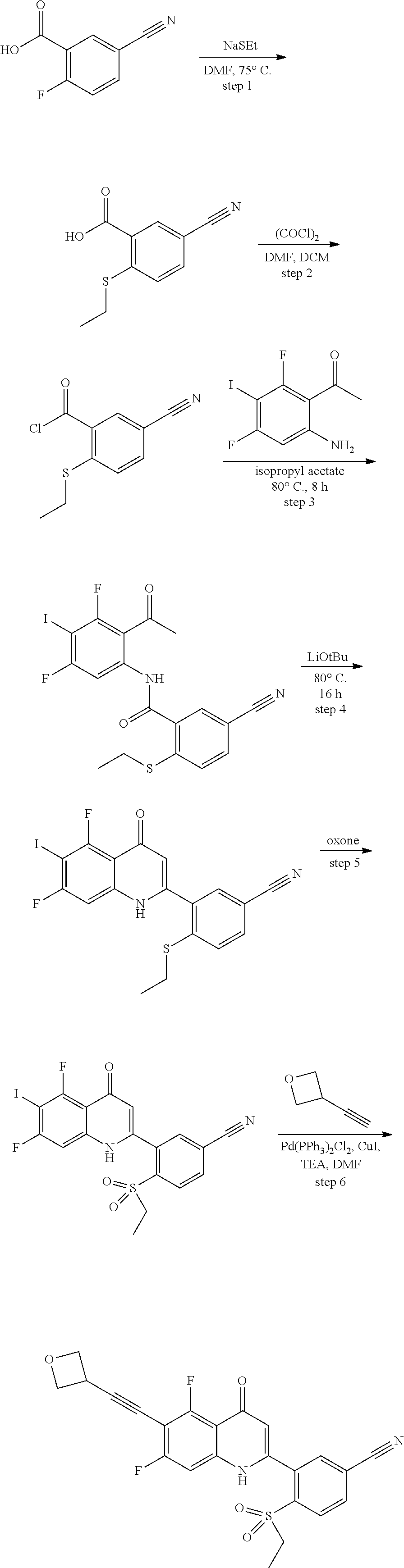

### Step 1: 5-cyano-2-(ethylthio)benzoic acid

Sodium ethanethiolate (1.8 g, 21.8 mmol) was added slowly to a mixture of 5-cyano-2-fluorobenzoic acid (2.0 g, 12.1 mmol) in DMF (25 mL) at 0° C. under N₂. The reaction mixture was stirred at 78° C. for 1 h. under N₂. The reaction mixture was diluted with water (120 mL) and adjusted pH to 2-3 with HCl aq. (2 N). The mixture was extracted with EA (120 mL×3). The combined organic layers were dried over with Na₂SO₄, filtered, and concentrated under reduced pressure to give the title compound (2.4 g, 95.0% yield) as a white solid. ¹H NMR (400 MHz, DMSO-d₆) δ 13.53 (s, 1H), 8.21 (d, J=2.0 Hz, 1H), 7.91 (dd, 1=8.5, 2.0 Hz, 1H), 7.57 (d, J=8.5 Hz, 1H), 3.02 (q, J=7.3 Hz, 2H), 1.29 (t, J=7.3 Hz, 3H).

### Step 2: 5-cyano-2-(ethylthio)benzoyl chloride

To a solution of 5-cyano-2-(ethylthio)benzoic acid (800 mg, 3.84 mmol) in DCM (25 mL) was added oxalyl chloride (0.96 g, 7.7 mmol) dropwise and DMF (1 drop) at 0° C. under N₂. The reaction mixture was stirred at r.t. for 1 h under N₂. The reaction mixture was concentrated under reduced pressure to give the crude product as a yellow solid which was used in next step.

### Step 3: N-(2-acetyl-3,5-difluoro-4-iodophenyl)-5-cyano-2-(ethylthio)benzamide

A mixture of 5-cyano-2-(ethylthio)benzoyl chloride (864.0 mg, 3.84 mmol) and 1-(6-amino-2,4-difluoro-3-iodophenyl)ethan-1-one (808.8 mg, 2.72 mmol) in isopropyl acetate (24 mL) was stirred at 80° C. overnight under N₂. The reaction mixture was concentrated under reduced pressure to give the crude product. The crude product was triturated with CH₃CN (6 mL) to give the desired product (500 mg, 37.8% yield) as an off-white solid. LCMS calc. for C₁₈H₁₄F₂IN₂O₂S [M+H]⁺: m/z=487.3; Found: 487.3.

### Step 4: 3-(5,7-difluoro-6-iodo-4-oxo-1,4-dihydroquinolin-2-yl)-4-(ethylthio)benzonitrile

A mixture of N-(2-acetyl-3,5-difluoro-4-iodophenyl)-5-cyano-2-(ethylthio)benzamide (400.0 mg, 0.84 mmol) and LiOtBu (78.8 mg, 0.96 mmol) in 2-methyltetrahydrofuran (7 mL) was stirred at 80° C. overnight. The reaction mixture was quenched with aq. HCl (0.1 mL, 2 N) and extracted with EtOAc (50 mL). The organic layers were concentrated under reduced pressure to give the crude product which was triturated with CH₃CN (5 mL) to give the desired product (340 mg, 68.5% yield) as a green solid. LCMS calc. for C₁₈H₁₂F₂IN₂OS [M+H]⁺: m/z=469.3; Found: 469.3.

### Step 5: 3-(5,7-difluoro-6-iodo-4-oxo-1,4-dihydroquinolin-2-yl)-4-(ethylsulfonyl)benzonitrile

To a mixture of 3-(5,7-difluoro-6-iodo-4-oxo-1,4-dihydroquinolin-2-yl)-4-(ethylthio)benzonitrile (280.0 mg, 0.60 mmol) in Acetone/H₂O/THF/MeOH (2.5 mL/1.8 mL/2.0 mL/2.0 mL) was added oxone (1.8 g, 3.0 mmol). The resulting mixture was stirred at 50° C. under N₂ overnight. The reaction was quenched with aq. Na₂SO₃. The reaction mixture was diluted with water (120 mL) and filtered. The filter cake washed with water and dried under reduce pressure. The crude product was triturated with MTBE (5 mL×2), and then the crude product was triturated with MeOH (5 mL×2) to give the desired product (170 mg, 56.6% yield) as a white solid. ¹H NMR (400 MHz, DMSO-d₆) δ 12.22 (s, 1H), 8.45-8.12 (m, 3H), 7.16 (d, J=8.4 Hz, 1H), 6.17 (s, 11H), 3.37-3.34 (m, 2H), 1.12 (t, J=7.2 Hz, 3H). LCMS calc. for C₁₈H₁₂F₂IN₂O₃S [M+H]⁺: m/z=501.2; Found: 501.2.

### Step 6: 3-(5,7-difluoro-6-(oxetan-3-ylethynyl)-4-oxo-1,4-dihydroquinolin-2-yl)-4-(ethylsulfonyl)benzonitrile

A mixture of 3-(5,7-difluoro-6-iodo-4-oxo-1,4-dihydroquinolin-2-yl)-4-(ethylsulfonyl)benzonitrile (50 mg, 0.1 mmol), 3-ethynyloxetane (12.3 mg, 0.15 mmol), CuI (2.0 mg, 0.1 mmol) and PdCl₂(PPh₃)₂ (7.1 mg, 0.01 mmol) in TEA (0.4 mL) and DMF (1.2 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mmi 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (24.4 mug, 53.7% yield) as a white solid. ¹H NMR (400 MHz, DMSO-d₆) δ 12.27 (s, 1H), 8.45-8.10 (m, 3H), 7.15 (s, 1H), 6.16 (s, 1H), 4.85 (dd, J=8.5, 5.5 Hz, 2H), 4.64 (dd, J=6.8, 5.7 Hz, 2H), 4.38-4.19 (m, 1H), 3.48-3.34 (m, 2H), 1.13 (t, J=7.1 Hz, 3H). LCMS calc. for C₂₃H₁₇F₂N₂O₄S [M+H]⁺: m/z=455.4; Found: 455.4.

### Example 47: 3-(6-((3-(Difluoromethoxy)pyridin-4-yl)ethynyl)-5,7-difluoro-4-oxo-1,4-dihydroquinolin-2-yl)-4-(methylsulfonyl)benzonitrile

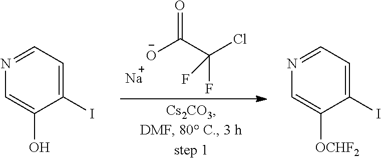

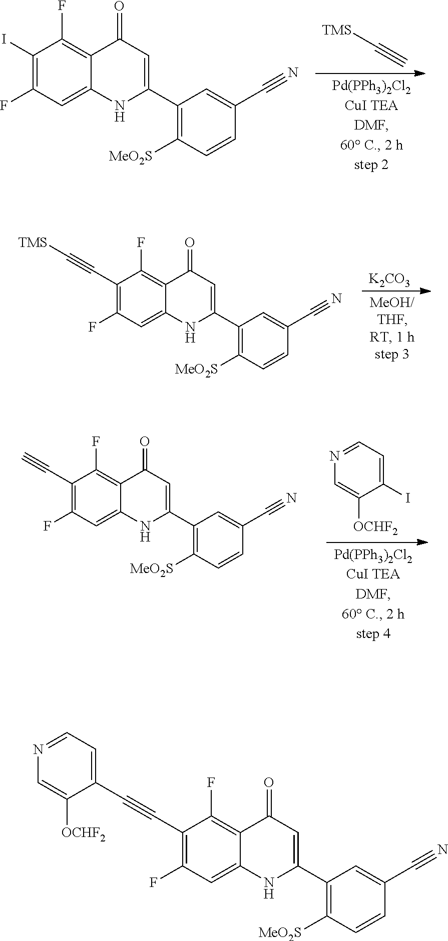

### Step 1: 3-(difluoromethoxy)-4-iodopyridine

To a solution of 4-iodopyridin-3-ol (500 mg, 2.262 mmol) in DMF (10 mL) was added Cs₂CO₃ (885 mg, 2.715 mmol) and sodium 2-chloro-2,2-difluoroacetate (414 mg, 2.715 mmol). The mixture was stirred at 80° C. for 3 h. The reaction mixture was diluted with water (100 mL) and extracted with EA (40 mL×3). The combined organic layers were washed with brine, dried over anhydrous Na₂SO₁, filtered and concentrated under reduced pressure. The residue was purified by flash chromatography on a silica gel column eluting with EA/PE (17%) to afford the title compound (63 mg, 10% yield) as a colorless oil. LCMS calc. for C₆H₅F₂INO [M+H]⁺: m/z=271.9; Found: 271.9.

### Step 2: 3-(5,7-difluoro-4-oxo-6-((trimethylsilyl)ethynyl)-1,4-dihydroquinolin-2-yl)-4-(methylsulfonyl)benzonitrile

A mixture of 3-(5,7-difluoro-6-iodo-4-oxo-1,4-dihydroquinolin-2-yl)-4-(methylsulfonyl)benzonitrile (100 mg, 0.206 mmol, Example 1 Step 5), ethynyltrimethylsilane (202 mg, 2.06 mmol), Cut (4 mg, 0.021 mmol) and PdCl₂(PPh₃)₂ (15 mg, 0.021 mmol) in TEA (0.5 mL) and DMF (1.5 mL) was degassed and recharged with nitrogen for 3 cycles, and then stirred at 60° C. for 2 h. The reaction mixture was diluted with water (15 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (2%) to afford the title compound (62 mg, 66% yield) as a yellow solid. LCMS calc. for C₂₂H₁₉F₂N₂O₃SSi [M+H]⁺: m/z=457.1; Found: 457.0.

### Step 3: 3-(6-ethynyl-5,7-difluoro-4-oxo-1,4-dihydroquinolin-2-yl)-4-(methylsulfonyl)benzonitrile

To a solution of 3-(5,7-difluoro-4-oxo-6-((trimethylsilyl)ethynyl)-1,4-dihydroquinolin-2-yl)-4-(methylsulfonyl)benzonitrile (62 mg, 0.136 mmol) in MeOH (1 mL) and THE (1 mL) was added K₂CO₃ (38 mg, 0.272 mmol). The mixture was stirred at r.t. for 1 h. The reaction mixture was diluted with water (20 mL) and extracted with EA (10 mL×3). The combined organic layers were washed with brine, dried over anhydrous Na₂SO₄, filtered and concentrated under reduced pressure. The residue was purified by flash chromatography on a silica gel column eluting with MeOH/DCM (2%) to afford the title compound (32 mg, 61% yield) as a white solid. LCMS calc. for C₁₉H₁₁F₂N₂O₃S [M+H]⁺: m/z=385.0; Found: 385.0.

### Step 4: 3-(6-((3-(difluoromethoxy)pyridin-4-yl)ethynyl)-5,7-difluoro-4-oxo-1,4-dihydroquinolin-2-yl)-4-(methylsulfonyl)benzonitrile

A mixture of 3-(6-ethynyl-5,7-difluoro-4-oxo-1,4-dihydroquinolin-2-yl)-4-(methylsulfonyl)benzonitrile (32 mg, 0.083 mmol), 3-(difluoromethoxy)-4-iodopyridine (45 mg, 0.167 mmol), CuI (2 mg, 0.008 mmol) and PdCl₂(PPh₃)₂ (6 mg, 0.008 mmol) in TEA (0.3 mL) and DMF (0.8 mL) was degassed and recharged with nitrogen for 3 cycles, and then stirred at 60° C. for 2 h. The reaction mixture was diluted with water (10 mL) and extracted with EA (10 mL×3). The combined organic layers were washed with brine, dried over Na₂SO₄, filtered and concentrated under reduced pressure. The residue was purified by flash chromatography on a silica gel column eluting with MeOH/DCM (3%) to afford the crude product which was further purified by Prep-HPLC to afford the title compound (1.5 mg, 3% yield) as a yellow solid. ¹H NMR (400 MHz, DMSO-d₆) δ 12.40 (s, 1H), 8.65 (s, 1H), 8.57 (d, J=4.9 Hz, 1H), 8.38-8.27 (m, 3H), 7.71 (d, J=4.9 Hz, 1H), 7.44 (t, J=72.9 Hz, 1H), 7.22 (s, 1H), 6.24 (s, 1H), 3.30 (s, 3H). LCMS calc. for C₂₅H₁₄F₄N₃O₄S [M+H]⁺: m/z=528.1; Found: 528.1.

### Example 48: 3-(5,7-Difluoro-6-(3-methyl-3-(methylsulfonyl)but-1-yn-1-yl)-4-oxo-1,4-dihydroquinolin-2-yl)-4-(methylsulfonyl)benzonitrile

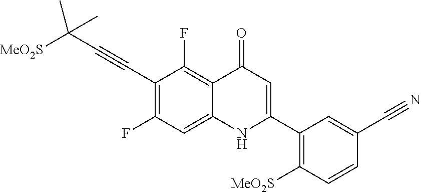 [SMILES: CC(C)(C#Cc1c(F)cc2[nH]c(-c3cc(C#N)ccc3S(C)(=O)=O)cc(=O)c2c1F)S(C)(=O)=O]

A mixture of 3-(5,7-difluoro-6-iodo-4-oxo-1,4-dihydroquinolin-2-yl)-4-(methylsulfonyl)benzonitrile (50 mg, 0.1 mmol, Example 1 Step 5), 3-methyl-3-(methylsulfonyl)but-1-yne (21.9 mg, 0.15 mmol), CuI (1.9 mg, 0.01 mmol) and PdCl₂(PPh₃)₂ (7.0 mg, 0.01 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered. and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (17.4 mg, 34.5% yield) as a white solid. ¹H NMR (400 MHz, DMSO-d₆) δ 12.32 (s, 1H), 8.47-8.10 (m, 3H), 7.16 (d, J=8.0 Hz, 1H), 6.20 (s, 1H), 3.31-3.26 (m, 3H), 3.21 (s, 3H), 1.69 (s, 3H). LCMS calc. for C₂₃H₁₉F₂N₂O₅S₂ [M+H]⁺: m/z=505.4; Found: 505.4.

### Example 49: 3-(5,7-Difluoro-6-(4-methoxybut-1-yn-1-yl)-4-oxo-1,4-dihydroquinolin-2-yl)-4-(methylsulfonyl)benzonitrile

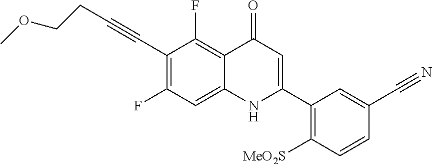 [SMILES: COCCC#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (50 mg, 0.1 mmol, Example 1 Step 5), 4-methoxybut-1-yne (12.6 mg, 0.15 mmol), CuI (1.9 mg, 0.01 mmol) and PdCl₂(PPh₃)₂ (7.0 mg, 0.01 mmol) in TEA (0.5 mL) and DMF (1.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (17.1 mg, 38.7% yield) as a white solid. ¹H NMR (400 MHz, DMSO-d₆) δ 12.24 (s, 1H), 8.47-8.15 (m, 3H), 7.12 (d, J=9.8 Hz, 1H), 6.16 (d, J=1.3 Hz, 1H), 3.55 (t, J=6.5 Hz, 2H), 3.32 (s, 3H), 3.30 (s, 3H), 2.79 (t, J=6.4 Hz, 2H). LCMS calc. for C₂₂H₁₇F₂N₂O₄S [M+H]⁺: m/z=443.1; Found: 443.4.

### Example 50: 3-(5,7-Difluoro-6-(3-methoxy-3-methylbut-1-yn-1-yl)-4-oxo-1,4-dihydroquinolin-2-yl)-4-(methylsulfonyl)benzonitrile

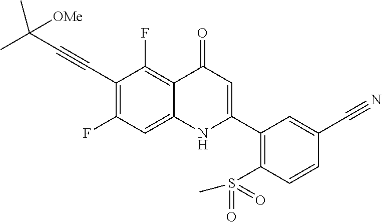 [SMILES: COC(C)(C)C#Cc1c(F)cc2[nH]c(-c3cc(C#N)ccc3S(C)(=O)=O)cc(=O)c2c1F]

A mixture of 3-(5,7-difluoro-6-iodo-4-oxo-1,4-dihydroquinolin-2-yl)-4-(methylsulfonyl)benzonitrile (25 mg, 0.051 mmol, Example 1 Step 5), 3-methoxy-3-methylbut-1-yne (7.6 mg, 0.077 mmol), CuI (1.0 mg, 0.0051 mmol) and PdCl₂(PPh₃)₂ (3.6 mg, 0.0051 mmol) in TEA (0.2 mL) and DMF (0.5 mL) was degassed and recharged with nitrogen for 3 cycles, and then stirred at 60° C. for 2 h. The reaction mixture was diluted with water (10 mL) and extracted with EA (10 mL×2). The combined organic layers were washed with brine, dried over Na₂SO₄, filtered, and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (4.09 mg, 17.6% yield) as a white solid. ¹H NMR (400 MHz, DMSO-d₆) δ 12.31 (s, 1H), 8.43-8.17 (m, 3H), 7.16 (d, J=9.6 Hz, 1H), 6.19 (s, 1H), 3.35 (s, 3H), 3.31 (s, 3H), 1.52 (s, 6H). LCMS calc. for C₂₃H₁₉F₂N₂O₄S [M+H]⁺: m/z=457.1; Found: 457.1.

### Example 51: 3-(5,7-Difluoro-6-((2-fluorophenyl)ethynyl)-4-oxo-1,4-dihydroquinolin-2-yl)-4-(methylsulfonyl)benzonitrile

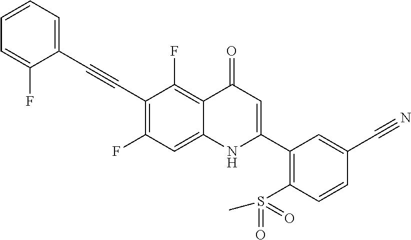 [SMILES: CS(=O)(=O)c1ccc(C#N)cc1-c1cc(=O)c2c(F)c(C#Cc3ccccc3F)c(F)cc2[nH]1]

A mixture of 3-(5,7-difluoro-6-iodo-4-oxo-1,4-dihydroquinolin-2-yl)-4-(methylsulfonyl)benzonitrile (50 mg, 0.103 mmol, Example 1 Step 5), 1-ethynyl-2-fluorobenzene (19 mg, 0.154 mmol), Pd(PPh₃)₂Cl₂ (7 mg, 0.01 mmol), CuI (2 mg, 0.01 mmol) and TEA (0.5 mL) in DMF (1 mL) was stirred at 60° C. for 2 h under N₂. The mixture was concentrated tinder reduced pressure. The residue was purified by Prep-HPLC on a C18 column eluting with MeCN/20 (0%-100%, with 0.1% NH₃·H₂O) to afford the title compound (19.7 mg, 40% yield) as a light yellow solid. ¹H NMR (400 MHz, DMSO-d₆) δ: 12.35 (s, 1H), 8.45-8.19 (m, 3H), 7.69 (td, J=7.5, 1.7 Hz, 111), 7.59-7.52 (m, 11H), 7.43-7.37 (m, 1H), 7.35-7.10 (in, 2H), 6.27 (s, 11H), 3.36 (s, 3H). LCMS calc. for C₂₅H₁₄F₃N₂O₃S [M+H]⁺: m/z=479.1; Found: 479.1.

### Example 52: 3-(5,7-Difluoro-4-oxo-6-((tetrahydro-2H-pyran-4-yl)ethynyl)-1,4-dihydroquinolin-2-yl)-4-(methylsulfonyl)benzonitrile

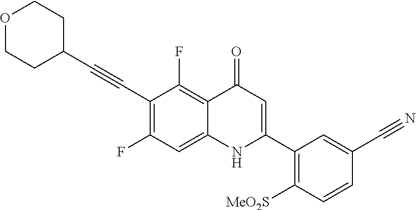 [SMILES: CS(=O)(=O)c1ccc(C#N)cc1-c1cc(=O)c2c(F)c(C#CC3CCOCC3)c(F)cc2[nH]1]

### Step 1: N-(2-acetyl-3,5-difluoro-4-((tetrahydro-2H-pyran-4-yl)ethynyl)phenyl)-5-cyano-2-(methylsulfonyl)benzamide

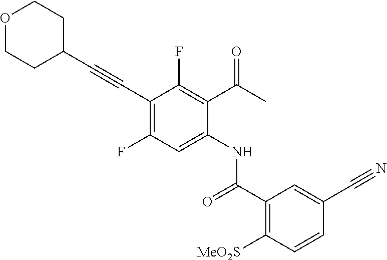 [SMILES: CC(=O)c1c(NC(=O)c2cc(C#N)ccc2S(C)(=O)=O)cc(F)c(C#CC2CCOCC2)c1F]

To a solution of 4-ethynyltetrahydro-2H-pyran (16.5 mg, 0.15 mmol), N-(2-acetyl-3,5-difluoro-4-iodophenyl)-5-cyano-2-(methylsulfonyl)benzamide (50 mg, 0.1 mmol, Example 1 Step 4), CuI (2 mg, 0.01 mmol) and PdCl₂(PPh₃)₂ (7 ng, 0.01 mmol) in TEA (0.5 mL) and DMF (0.5 mL) was degassed and recharged with nitrogen for 3 cycles, and then stirred at 70° C. for 2 h. The resulting mixture was concentrated under reduced pressure. The residue was purified by flash chromatography on a C18 column eluting with ACN/H₂O (5-14%) to afford the title compound (38 mg) as an off-white solid. LCMS calc. for C₂₄H₂₁F₂N₂O₅S [M+H]⁺: m/z=486.1; Found: 486.0.

### Step 2: 3-(5,7-difluoro-4-oxo-6-((tetrahydro-2H-pyran-4-yl)ethynyl)-1,4-dihydroquinolin-2-yl)-4-(methylsulfonyl)benzonitrile

A mixture of N-(2-acetyl-3,5-difluoro-4-(3-hydroxyprop-1-yn-1-yl)phenyl)-5-cyano-2-(methylsulfonyl)benzamide (38 mg, 0.08 mmol) and LiOtBu (12.8 mg, 0.16 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with ACN/H₂O (15-35% with 0.1 NH₄CO₃) to afford the title compound (3.8 mg) as an off-white solid. LCMS calc. for C₂₄H₁₇F₂N₂O₄S [M−H]⁻: m/z=467.1; Found: 467.0.

### Example 53: (E)-3-(5,7-Difluoro-4-oxo-6-styryl-1,4-dihydroquinolin-2-yl)-4-(methylsulfonyl)benzonitrile

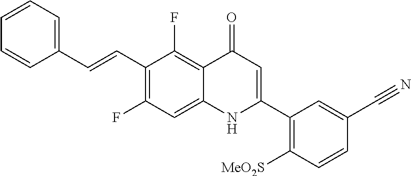 [SMILES: CS(=O)(=O)c1ccc(C#N)cc1-c1cc(=O)c2c(F)c(/C=C/c3ccccc3)c(F)cc2[nH]1]

### Step 1: (E)-N-(2-acetyl-3,5-difluoro-4-styrylphenyl)-5-cyano-2-(methylsulfonyl)benzamide

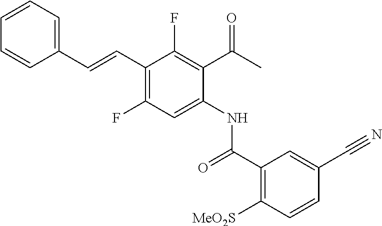 [SMILES: CC(=O)c1c(NC(=O)c2cc(C#N)ccc2S(C)(=O)=O)cc(F)c(/C=C/c2ccccc2)c1F]

A mixture of (E)-4,4,5,5-tetramethyl-2-styryl-1,3,2-dioxaborolane (26 mg, 0.3 mmol), Pd(dppf)Cl₂ (14 mg, 0.02 mmol), K₂CO₃ (68 mg, 0.5 mmol) and N-(2-acetyl-3,5-difluoro-4-iodophenyl)-5-cyano-2-(methylsulfonyl)benzamide (100 mg, 0.2 mmol, Example 1 Step 4) in 1,4-dioxane (1 mL) and water (0.1 mL) was degassed and recharged with nitrogen for 3 cycles, and then stirred at 100° C. overnight. The resulting mixture was concentrated under reduced pressure. The residue was purified by prep-TLC on silica gel eluting with EtOAc/PE (30%) to afford the title compound (18 mg) as a yellow solid. LCMS calc. for C₂₅H₁₉F₂N₂O₄S [M+H]⁺: m/z=481.1; Found: 481.1.

### Step 2: (E)-3-(5,7-difluoro-4-oxo-6-styryl-1,4-dihydroquinolin-2-yl)-4-(methylsulfonyl)benzonitrile

A mixture of (E)-N-(2-acetyl-3,5-difluoro-4-styrylphenyl)-5-cyano-2-(methylsulfonyl)benzamide (52 mg, 0.11 mmol) and LiOtBu (17.6 mg, 0.22 mmol) in 2-methyltetrahydrofuran (1 mL) was stirred at 75° C. overnight. The reaction was quenched with aq. HCl (0.1 mL, 2.0 M) and concentrated under reduced pressure. The residue was purified by flash chromatography on a C18 column eluting with MeCN/H₂O (50-55% with 0.1 NH₄CO₃) to afford the title compound (6.5 mg) as an off-white solid. LCMS calc. for C₂₅H₁₅F₂N₂O₃S [M−H]⁻: m/z=461.1; Found: 461.1.

### Example 54: 3-(5,7-Difluoro-4-oxo-1,4-dihydroquinolin-2-yl)-4-((1-methyl-1H-tetrazol-5-yl)thio)benzonitrile

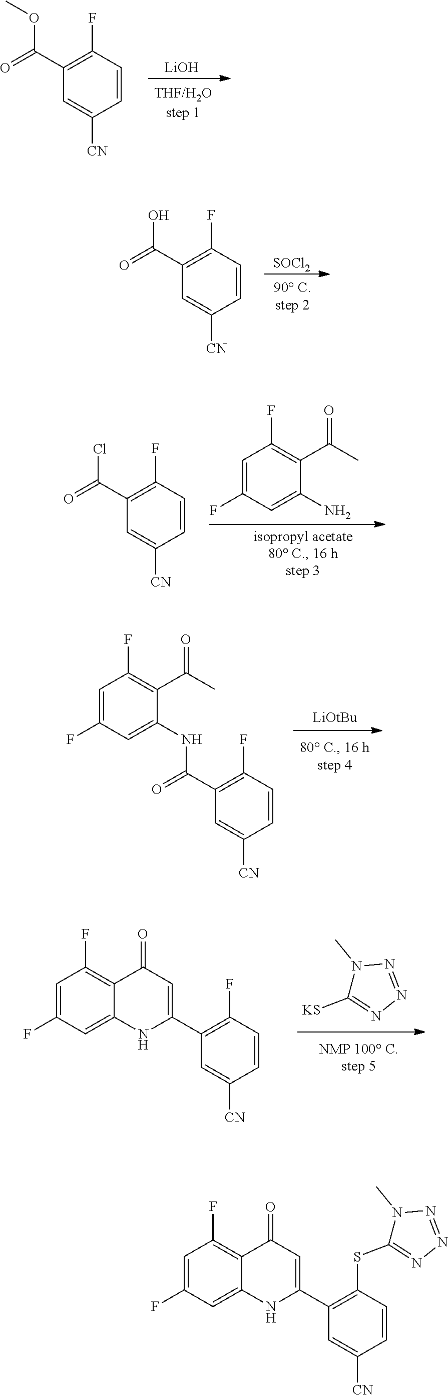

### Step 1: 5-cyano-2-fluorobenzoic acid

A mixture of methyl 5-cyano-2-fluorobenzoate (2.0 g, 11.2 mmol) and LiOH·H₂O (936.6 mg, 22.4 mmol) in THF/H₂O (15 mL/3 mL) was stirred at r.t. overnight. The reaction mixture was diluted with water (8 mL) and adjusted pH to 2-3 with HCl aq. (2 N). The mixture was extracted with DCM (30 mL×3). The combined organic layers were dried over with Na₂SO₄, filtered and concentrated under reduced pressure to give the title compound (1.3 g, 70.3% yield) as white solid. LCMS calc. for C₁₈H₂₂N₂O₅ [M−H]⁻: m/z=163.9; Found: 163.9.

### Step 2: 5-cyano-2-fluorobenzoyl chloride

A mixture of 5-cyano-2-fluorobenzoic acid (600 mg, 3.64 mmol) in SOCl₂ (10 mL) was degassed and recharged with nitrogen for 3 cycles, and then stirred at 90° C. for 2 h. The reaction mixture was concentrated under reduced pressure to give the crude product as a white solid which was used in next step without further purification.

### Step 3: N-(2-acetyl-3,5-difluorophenyl)-5-cyano-2-fluorobenzamide

A mixture of 5-cyano-2-fluorobenzoyl chloride (880 mug, 4.87 mmol) and 1-(2-amino-4,6-difluorophenyl)ethan-1-one (500 mg, 2.92 mmol) in isopropyl acetate (5 mL) was stirred at 80° C. overnight under N₂. The reaction mixture was diluted with EtOAc (50 mL). The organic layers were washed with water and brine, dried over Na₂SO₄, filtered and concentrated under reduced pressure. The residue was crystallized with EA/PE=1/4 to afford the title compound (326 mg, 35% yield) as an off-white solid. LCMS calc. for C₁₆H₁₀F₃N₂O₂ [M+H]⁺: m/z=319.1; Found: 319.1.

### Step 4: 3-(5,7-difluoro-4-oxo-1,4-dihydroquinolin-2-yl)-4-fluorobenzonitrile

A mixture of N-(2-acetyl-3,5-difluorophenyl)-5-cyano-2-fluorobenzamide (325 mg, 1.02 mmol) and LiOtBu (130.8 mg, 1.635 mmol) in 2-methyltetrahydrofuran (10 mL) was stirred at 75° C. overnight. The reaction mixture was quenched with aq. HCl (0.1 mL, 2 M) and concentrated under reduced pressure. The residue was diluted with EtOAc (50 mL). The organic layers were washed with water and brine, dried over Na₂SO₄, filtered and concentrated under reduced pressure. The residue was crystallized with EA/PE=1:4 to afford the title compound (196 mg, 64% yield) as a yellow solid. LCMS calc. for C₁₆H₈F₃N₂O [M+H]⁺: m/z=301.2; Found: 301.2.

### Step 5: 3-(5,7-difluoro-4-oxo-1,4-dihydroquinolin-2-yl)-4-((7-methyl-1H-tetrazol-5-yl)thio)benzonitrile

A mixture of 3-(5,7-difluoro-4-oxo-1,4-dihydroquinolin-2-yl)-4-fluorobenzonitrile (140.0 mg, 0.47 mmol) and potassium 1-methyl-1H-tetrazole-5-thiolate (86.8 mg, 0.56 mmol) in NMP (3 mL) was stirred at 100° C. overnight under N₂. The reaction mixture was diluted with water (30 mL) and extracted with EA (30 mL×2). The combined organic layers were washed with brine, dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (17.2 mg, 9.2% yield) as a white solid. ¹H NMR (400 MHz, DMSO-d₆) ¹H NMR (400 MHz, DMSO) δ 12.19 (s, 11H), 8.23 (d, J=1.7 Hz, 1H), 7.96 (s, 1H), 7.47 (s, 1H), 7.19 (s, 2H), 6.14 (s, 1H), 3.95 (s, 3H). LCMS calc. for C₁₈H₁₁F₂N₆OS [M+H]⁺: m/z=397.1; Found: 397.4.

### Example 55: 3-(5,7-Difluoro-6-(3-fluoro-3-methylbut-1-yn-1-yl)-4-methoxyquinolin-2-yl)-4-(methylsulfonyl)benzonitrile

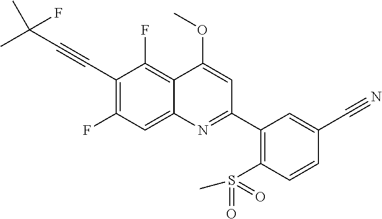 [SMILES: COc1cc(-c2cc(C#N)ccc2S(C)(=O)=O)nc2cc(F)c(C#CC(C)(C)F)c(F)c12]

To a solution of 3-(5,7-difluoro-6-(3-hydroxy-3-methylbut-1-yn-1-yl)-4-oxo-1,4-dihydroquinolin-2-yl)-4-(methylsulfonyl)benzonitrile (30.0 mg, 0.07 mmol, Example 1 Step 6) in DCM (3.5 mL) was added DAST (21.9 mg, 0.14 mmol) dropwise at 0° C. under N₂. The mixture was stirred at r.t. for 1 h. The reaction mixture was diluted with DCM (5 mL) and poured into cold sat. NaHCO₃ (aq.) (10 mL). The mixture was extracted with DCM (10 mL×2). The combined organic layers were dried over Na₂SO₄, filtered and concentrated under reduced pressure. The residue was purified by prep-HPLC (Instrument: GILSON-281 Column: XBridge C18 250*19.00 mml 10 um Temperature: 25° C. Mobile phase: A: H₂O (10 mM NH₄HCO₃) B: ACN Flow rate: 20.0 ml/min Gradient: 30-60 Detection wavelength: 214/254 nm Retention time: 6-14 min) to afford the title compound (1.0 mg, 3.9% yield) as a white solid. ¹H NMR (400 MHz, DMSO-d₆) δ 8.35-8.24 (m, 3H), 7.81 (d, J=10.3 Hz, 1H), 7.43 (s, 1H), 4.12 (s, 3H), 3.65 (s, 3H), 1.80 (s, 3H), 1.75 (s, 3H). LCMS calc. for C₂₃H₁₈F₃N₂O₃S [M+H]⁺: m/z=459.1; Found: 459.3.

### Example 56: 5,7-Difluoro-2-(5-methyl-2-(methylsulfonyl)phenyl)-6-(2-(tetrahydro-2H-pyran-4-yl)ethyl)quinolin-4(1H)-one

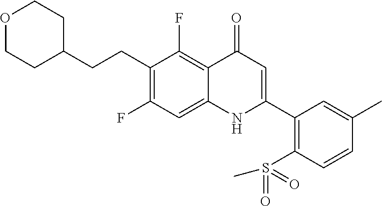 [SMILES: Cc1ccc(S(C)(=O)=O)c(-c2cc(=O)c3c(F)c(CCC4CCOCC4)c(F)cc3[nH]2)c1]

To a mixture of 3-(5,7-difluoro-4-oxo-6-((tetrahydro-2H-pyran-4-yl)ethynyl)-1,4-dihydroquinolin-2-yl)-4-(methylsulfonyl)benzonitrile (25.0 mg, 0.05 mmol, Example 52 Step 2) and 10% Pd/C (70.0 mg) in MeOH (4 mL) was stirred at r.t. under H₂ for 18 h. The reaction mixture was diluted with MeOH (5 mL) and filtered by a pad of celite. The filtrate was concentrated under reduced pressure to give a crude product which was purified by flash chromatography on a silica gel column eluting with DCM/MeOH (5%) to afford the title compound (3.93 mg, 17.0% yield) as a white solid. ¹H NMR (400 MHz, CDCl₃) δ 7.95 (d, J=7.9 Hz, 1H), 7.50-7.37 (m, 2H), 7.03 (s, 1H), 6.25 (s, 1H), 3.97 (d, J=8.8 Hz, 2H), 3.38 (t, J=11.4 Hz, 2H), 3.00 (s, 3H), 2.76 (s, 2H), 2.49 (s, 3H), 1.69 (d, J=12.7 Hz, 2H), 1.62-1.46 (m, 3H), 1.43-1.29 (m, 2H). LCMS calc. for C₂₄H₂₆F₂NO₄S [M+H]⁺: m/z=462.1; Found: 462.3.

### Example A: PPARγ-SMRT Recruitment Assay

PPARγ TR-FRET assay was initiated to evaluate potential activatory effect of the compounds of the present disclosure for SMRT recruitment to PPARγ protein. PPARγ-LBD (UniProt ID: P37231-2) was expressed and purified in insect system and stored at −80° C. in aliquots. Biotinylated SMRT peptide was synthesized (Biotin-HASTNMGLEAIIRKALMGKYDQW). 5 μL protein solution mixture consisting of 10 nM PPARγ-LBD and 2 nM Anti-His Eu (Cisbio, 61HISKLA) in assay buffer (25 mM HEPES pH7.4, 25 mM KCl, 1 mM EDTA, 0.01% BSA, 0.01% Tween-20, 1 mM TCEP) was added to 384-well plates containing duplicate 10-point dose response titration of compounds in 5 μL assay buffer (1% f.c. DMSO(v/v)). After 120 min pre-incubation at room temperature, 5 μL peptide solution mixture consisting of 50 nM Biotin-SMRT peptide and 25 nM Streptavidin-XL665 (Cisbio, 610SAXLA) added to plates. Plates were incubated for 60 min. and read in CLARIOstar Plus plate reader with Ex/Em 620 nM/665 nM. Average TR-FRET ratio of high control (Wells with 10 uM control compound) was calculated as High Control (HC). Average TR-FRET ratio of low control (Wells with 1% DMSO) was calculated as Low control (LC). For one compound, top % is the max value of % Activation.

$$\% Activision = 100 * ( {Ratio}_{cmpd} - {Ratio}_{Ave \_ LC} ) / ( {Ratio}_{Ave \_ HC} - {Ratio}_{Ave \_ LC} ) .$$

EC₅₀ values were determined by fitting the data to the standard 4 parameters with Hill Slope using GraphPad Prism software. EC₅₀ data is proved below: a “+” denotes an EC₅₀ value of >1 μM, a “++” denotes an EC₅₀ value of 0.1 μM<EC₅₀<=1 μM, and a “+++” denotes an EC₅₀ value of <=0.1 μM. ND: No Determine.

TABLE 1
PPARγ binding assay

| Example | SMRT EC₅₀ | Top % | Example | SMRT EC₅₀ | Top % |
|---|---|---|---|---|---|
| 1 | +++ | 495% | 2 | +++ | 311% |
| 3 | +++ | 300% | 4 | +++ | 200% |
| 5 | +++ | 367% | 6 | +++ | 266% |
| 7 | +++ | 207% | 8 | +++ | 208% |
| 9 | +++ | 359% | 10 | +++ | |
| 11 | +++ | 435% | 12 | +++ | 181% |
| 13 | +++ | 187% | 14 | | |
| 15 | | | 16 | | |
| 17 | +++ | 613% | 18 | +++ | |
| 19 | +++ | 208% | 20 | +++ | 210% |
| 21 | +++ | | 22 | +++ | |
| 23 | +++ | 316% | 24 | +++ | 197% |
| 25 | +++ | | 26 | +++ | |
| 27 | +++ | | 28 | +++ | 293% |
| 29 | +++ | 357% | 30 | +++ | 283% |
| 31 | +++ | 181% | 32 | +++ | 343% |
| 33 | +++ | 502% | 34 | +++ | 290% |
| 35 | +++ | 243% | 36 | +++ | 322% |
| 37 | +++ | 406% | 38 | +++ | |
| 39 | +++ | 406% | 40 | +++ | 237% |
| 41 | +++ | | 42 | +++ | 231% |
| 43 | +++ | 237% | 44 | +++ | 286% |
| 45 | +++ | 249% | 46 | +++ | 174% |
| 47 | +++ | 376% | 48 | +++ | |
| 49 | +++ | | 50 | +++ | 372% |
| 51 | +++ | 447% | 52 | +++ | |
| 53 | +++ | 650% | 54 | + | |
| 55 | + | | 56 | ++ | |

### Example B: PPARγ-DRIP205 Blockade Assay

PPARγ TR-FRET assay was initiated to evaluate potential blockade effect of the compounds of the present disclosure for DRIP205 binding to PPARγ protein, PPARγ-LBD (UniProt ID: P37231-2) was expressed and purified in insect system and stored at −80° C. in aliquots. Biotinylated DRIP205 peptide was synthesized (Biotin-GNTKNHPMLMNLLKDNPADQF). 5 μL protein solution mixture consisting of 10 nM PPARγ-LBD and 2 nM Anti-His Eu (Cisbio, 61HISKLA) in assay buffer (25 mM HEPES pH7.4, 25 mM KCl, 1 mM EDTA, 0.01% BSA, 0.01% Tween-20, 1 mM TCEP) was added to 384-well assay plate containing duplicate 10-point dose response titration of compounds in 2.5 μL assay buffer (1% f.c. DMSO(v/v)) and 500 nM Rosiglitazone in 2.5 μL assay buffer. After 120 min pre-incubation at room temperature, 5 μL peptide solution mixture consisting of 50 nM Biotin-DRIP205 peptide and 25 nM Streptavidin-XL665 (Cisbio, 610SAXLA) was added to assay plate. Assay plate was incubated for 60 min and read in CLARIOstar Plus plate reader with Ex/Em 620 nM/665 nM. Average TR-FRET ratio of high control (Wells with 500 nM Rosiglitazone) was calculated as High Control (HC). Average TR-FRET ratio of low control (Wells with 1% DMSO) was calculated as Low control (LC).

$$\% Inhibition = 100 - 100 * ( {Ratio}_{cmpd} - {Ratio}_{Ave \_ LC} ) / ( {Ratio}_{Ave \_ HC} - {Ratio}_{Ave \_ LC} ) .$$

IC₅₀ values were determined by fitting the data to the standard 4 parameters with Hill Slope using GraphPad Prism software. IC₅₀ data is proved below: a “+” denotes an IC₅₀ value of >5 μM, a “++” denotes an IC₅₀ value of 0.5 μM<IC₅₀<=5 μM, and a “+++” denotes an IC₅₀ value of <=0.5 μM.

### Example C: Cell Based PPARγ Reporter Assay

PPARγ reporter assay was initiated to evaluate potential inhibitory effect of the compounds of the present disclosure for nuclear receptor activation. HEK293T cells were seeded in 96-well plates at 2.5×10⁴ cells/well in 100 μL RPMI 1640 containing 10% FBS, then transfected with GAL4-UAS-Luciferase (Addgene) reporter plasmids (50 ng/well) and pcDNA3.1-Gal4 DBD-PPARγ LBD (BGI) expression plasmids (50 ng/well) using FuGENE HD (Promega; 0.3 μL/well). After incubation for 18 h, fresh culture medium containing 300 nM Rosiglitazone as positive control instead of cell supernatant, 0.1% DMSO as a negative control. Compounds dissolved in DMSO were plated in duplicate, and tested on a 9-point, 3-fold serial dilution. DMSO final concentration was 0.2% for all wells. After 24 h compound treatment, activity was determined by the Luciferase Assay System (Promega).

Average luminescence signal of high control (Wells with 300 nM Rosiglitazone) was calculated as High Control (HIC). Average luminescence signal of low control (Wells with 0.1% DMSO) was calculated as Low control (LC).

$$\% Inhibition = 100 - 100 * ( {Signal}_{cmpd} - {Signal}_{Ave \_ LC} ) / ( {Signal}_{Ave \_ HC} - {Signal}_{Ave \_ LC} ) .$$

### Example D: Cell Viability Assay

Cell viability studies were conducted in Bladder cancer cell lines 5637 which is PPARγ amplified cell line, and PPARγ low expression cell line SW1710 was included as a control. Cells were cultured in RPMI 1640 supplemented with 10% FBS and 1% penicillin-streptomycin (Gibco, 15140122). Cells were seeded in 96-well cell culture plates (PerkinElmer, 6005680) at a density of 400 cells/well for 5637 cell and 600 cells/well for SW1710 cell. Compounds of the present disclosure dissolved in DMSO were plated in duplicate using a multichannel pipette, and tested on a 9-point 3-fold serial dilution. The final concentration of DMSO was 0.2% in all wells. Cells were incubated for 6 days in a 37° C., 5% CO₂ humidified incubator at. Cell viability was measured using the Cell Titer-Glo reagent (Promega, Catalog #: G7573) according to the manufacturer's instructions, luminescence signal was measured with a multimode plate reader (BMG CLARIO star plus). Average values of 0.2% DMSO-treated wells on each plate were calculated as high control (HC), while average values of only medium on each plate was calculated as low control (LC).

The percent inhibition was calculated using the formula: % Inhibition=100−100*(Signal_{cmpd}−Signal_{Ave_LC})/(Signal_{Ave_HC}−Signal_{Ave_LC}).

Although the present invention has been comprehensively described through its embodiments, it is worth noting that various changes and modifications are obvious to those skilled in the art. Such changes and modifications should be included in the scope of the appended claims of the present invention.

What claimed is:

1. A compound of formula (I), or a pharmaceutically acceptable salt, stereoisomer, solvate, N-oxide, tautomer, isotopic variant, prodrug, or deuterated compound thereof, wherein: 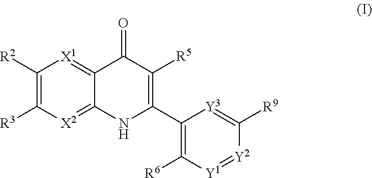
or a pharmaceutically acceptable salt, stereoisomer, solvate, N-oxide, tautomer, isotopic variant, prodrug, or deuterated compound thereof, wherein:
X¹ is N or CR¹;
X² is N or CR⁴;
Y¹ is N or CR⁷;
Y² is N or CR⁸;
Y³ is N or CR¹⁰;
R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D}, C(O)OR^{A}, OC(O)R^{B}, OC(O)NR^{C}R^{D}NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a};
R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}; and R² is not CH₃;
each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂R^{C}R^{D}, S(O)(═NR^{C})R^{B} or NR^{C}S(O)(=NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂;
Cy is phenyl, C₃-C₁₀ cycloalkyl, 5-10 membered heteroaryl, 4-10 membered heterocycloalkyl; wherein, the phenyl, C₃-C₁₀ cycloalkyl, 5-10 membered heteroaryl, 4-10 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a} C(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, S(O)₂NR^{c}R^{d};
Y is C₁-C₄ alkylene, C₃-C₆ alkenylene;
Z is CN, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(═NR^{C})R^{B} or NR^{C}S(O)(═NR^{C})R^{B};
R³ is H, D, halogen, CN, C₁-C₄ alkyl, C₁-C₄haloalkyl; or
R¹ and R² or R² and R³ together with the atoms to which they are attached form phenyl, C₅-C₆ cycloalkyl, 5-6 membered heteroaryl, 5-6 membered heterocycloalkyl; wherein, the phenyl, C₅-C₆ cycloalkyl, 5-6 membered heteroaryl, 5-6 membered heterocycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a};
R⁴ is H, D, halogen, CN;
R⁵ is selected from H, D, halogen, OH, CH₃, CF₃, CHF₂, CH₂F;
R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂NR^{6A}R^{6B}, S(O)(=NR^{6A})R^{6B};
R^{6A} and R^{6B} are each independently selected from H, C₁-C₄ alkyl, C₁-C₄ haloalkyl, C₁-C₄ alkyl-OH, C₁-C₄ alkyl-OC₁-C₄ alkyl, C₁-C₄ alkyl-NH₂, C₁-C₄ alkyl-NHC₁-C₄ alkyl, C₁-C₄ alkyl-N(C₁-C₄ alkyl)₂, C₃-C₄ cycloalkyl optionally substituted by halogen, phenyl optionally substituted by C₁-C₄ alkyl, C₁-C₄ haloalkyl;
R⁷ and R¹⁰ are each independently selected from H, D;
R⁸ is selected from H, D, halo, OH, C₁-C₄ alkyl, C₁-C₄ haloalkyl;
R⁹ is selected from CF₃, SF₅, CN, NC, NO₂;
or R⁸ and R⁹ together with the atoms to which they are attached form 5-membered heteroaryl optionally substituted by oxo;
R^{A} and R^{a} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄alkyl)₂;
R^{B} and R^{b} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄alkyl, C₁-C₄ haloalkyl, OC₁-C₄ alkyl, NHC₁-C₄ alkyl, N(C₁-C₄ alkyl)₂;
R^{C}, R^{D}, R^{c} and R^{d} are each independently selected from H, D, C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl; wherein, the C₁-C₆ alkyl, C₃-C₁₀ cycloalkyl-C₀-C₄ alkyl, 4-6 membered heterocycloalkyl-C₀-C₄ alkyl, phenyl-C₀-C₄ alkyl, or 5-6 membered heteroaryl-C₀-C₄ alkyl is optionally substituted with 1, 2, 3, 4 or 5 substituents independently selected from D, CN, halo, oxo, OH, NH₂, C₁-C₄ alkyl, C₁-C₄ haloalkyl, OC₁-C₄ alkyl, NHC₁-C₄ alkyl, N(C₁-C₄ alkyl)₂;
or R^{C} and R^{D} or R^{c} and R^{d} together with the N atom to which they are attached form a 4-6 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, C₁-C₄ cyanoalkyl, OC₁-C₄ alkyl, or OC₁-C₄ haloalkyl;
R^{E} and R^{e} are each independently selected from H, D, C₁-C₄ alkyl, C₁-C₄ haloalkyl, or (C₁-C₄ alkoxy)-C₁-C₄ alkyl;
R^{F} and R^{f} are each independently selected from H, D, C₁-C₄ alkyl, C₁-C₄ haloalkyl, or (C₁-C₄ alkoxy)-C₁-C₄ alkyl;
R^{G}, R^{H}, R^{I}, R^{g}, R^{h} and Rⁱ are each independently selected from C₁-C₄ alkyl or phenyl.

2. The compound of claim 1, wherein, X¹ is CR¹ and X² is CR⁴; X¹ is CR¹ and X² is N; X¹ is N and X² is CR⁴; and/or
R¹ is selected from H, D, halogen, CN, C₁-C₆ alkyl, C₂-C₆ alkenyl, C₂-C₆ alkynyl, C₃-C₆ cycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}; wherein, the C₁-C₆ alkyl, C₂-C₆ alkenyl, C₂-C₆ alkynyl, C₃-C₆ cycloalkyl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}.

3. The compound of claim 1, wherein, R² is selected from D, NO₂, N₃, SF₅, C₁-C₆ alkyl, C₂-C₆ alkenyl, C₂-C₆ alkynyl, C₃-C₁₀ cycloalkyl, 4-10 membered heterocycloalkyl having a heteroatom selected from Si, O—C₀-C₄ alkyl-Cy, O—Y—Z, C₀-C₄ alkyl-OCy, C₀-C₄ alkyl-NR^{C}Cy, C₀-C₄ alkyl-SCy, C₀-C₄ alkyl-C(O)Cy, C₀-C₄ alkyl-C(O)OCy, C₀-C₄ alkyl-C(O)NR^{C}Cy, NR^{C}COCy, NR^{C}CO₂Cy, NR^{C}C(S)OCy, NR^{C}C(O)NR^{C}Cy, NR^{C}C(S)NR^{C}Cy, —NR^{C}SO₂NR^{C}Cy, C(S)Cy, C(S)OCy, C(S)NR^{C}Cy, NR^{C}C(S)Cy, SOCy, SO₂Cy, SONR^{C}Cy, SiR^{G}R^{H}R^{I}, B(OR^{C})(OR^{D}), P(O)R^{E}R^{F}, P(O)OR^{E}OR^{F}, OP(O)OR^{E}OR^{F}; wherein, the C₁-C₆ alkyl, C₂-C₆ alkenyl, C₂-C₆ alkynyl, C₃-C₁₀ cycloalkyl, 4-10 membered heterocycloalkyl having a heteroatom selected from Si is optionally substituted by 1, 2, 3, 4, or 5 substituents independently selected from R^{2A}; preferably, R² is selected from D, NO₂, N₃, SF₅, CH₂CN, CH₂CH₂CN, CH(OH)CF₃, CH(OCH₃)CF₃, cyclopropyl, 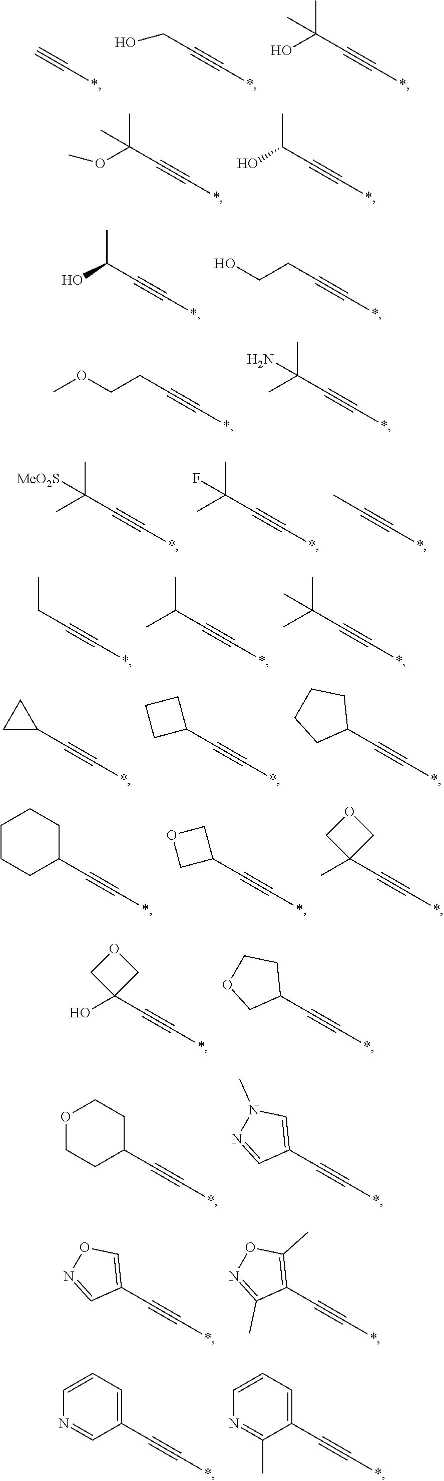 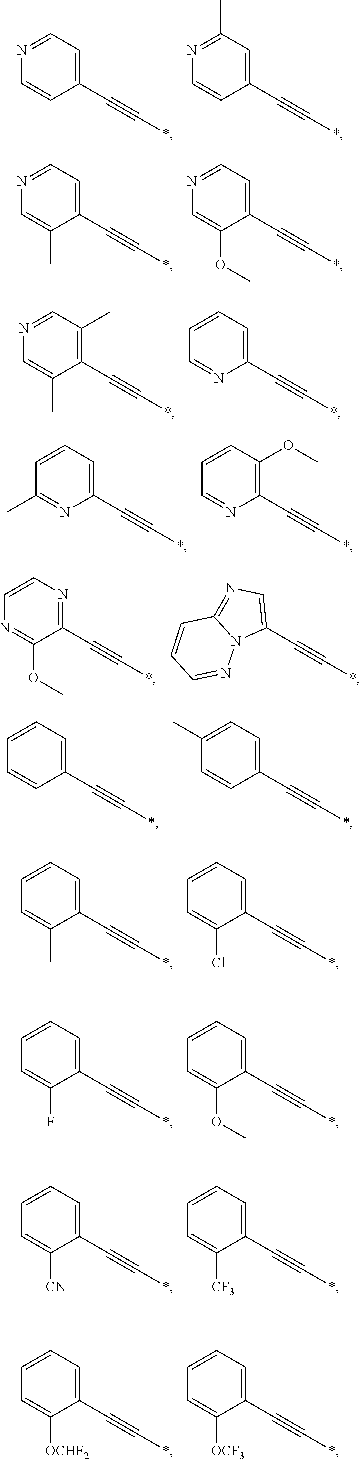 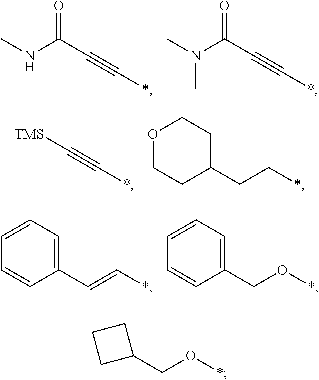
preferably, each R^{2A} is independently selected from H, D, halo, CN, oxo, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, SiR^{G}R^{H}R^{I}, B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}R^{F}, OP(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, S(O)(=NR^{C})R^{B} or NR^{C}S(O)(=NR^{C})R^{B}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, C(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(=NR^{c})^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂.

4. The compound of claim 1, wherein, R³ is H, D, halogen, CN, C₁-C₄ alkyl, C₁-C₄ haloalkyl; or
R² and R³ together with the atoms to which they are attached form phenyl, 5-6 membered heteroaryl; wherein, the phenyl, 5-6 membered heteroaryl is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)R^{c}R^{d}, NR^{c}(O)OR^{a}; preferably, R² and R³ together with the atoms to which they are attached form phenyl, pyrrolyl, imidazolyl, pyrazolyl; each is optionally substituted by 1, 2, 3, 4, or 5 substituents independently selected from D, halo, CN, oxo, C₁-C₆ alkyl, C₁-C₆ haloalkyl, C₁-C₆ alkyl-OH, C₁-C₆ alkyl-NH₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)NR^{c}R^{d}, NR^{c}C(O)OR^{a}; or
R² and R³ together with the atoms to which they are attached form phenyl, 1-methyl-1H-pyrazolyl.

5. The compound of claim 1, wherein, R⁴ is H, D, halogen, CN; and/or
R⁵ is selected from H, D, halogen, OH, CH₃, CF₃, CHF₂, CH₂F; and/or
R⁶ is selected from halogen, OS(O)₂R^{6A}, SR^{6A}, S(O)R^{6A}, S(O)OR^{6A}, S(O)₂R^{6A}, S(O)₂OR^{6A}, S(O)₂NR^{6A}R^{6B}, S(O)(=NR^{6A})R^{6B}.

6. The compound of claim 1, wherein, R⁶ is F, Cl, Br, I, SCH₃, S(O)CH₃, S(O)₂CH₃, S(O)₂CH₂CH₃, S(O)₂C₁H(CH₃)₂, S(O)₂CH₂CH(CH₃)₂, S(O)₂CF₃, S(O)₂CH₂CF₃, OS(O)₂CH₃, OS(O)₂CF₃, OTs,

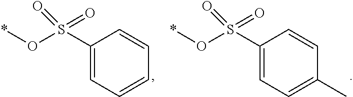

7. The compound of claim 1, wherein, Y¹ is N; or
Y¹ is CR⁷; and R² is H, D; preferably, Y¹ is CH; or
Y² is N; or
Y² is CR⁸; and R⁸ is U, D, halo, OH, C₁-C₄ alkyl, C₁-C₄ haloalkyl.

8. The compound of claim 1, wherein, R^{C} is selected from CF₃, SF₅, CN, NC, NO₂; or
R⁸ and R⁹ together with the atoms to which they are attached form 5-membered heteroaryl optionally substituted by oxo; preferably, R⁸ and R⁹ together with the atoms to which they are attached form 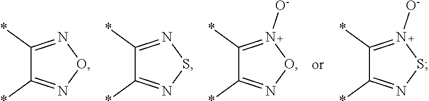
and/or
Y³ is N; or
Y³ is CR¹⁰; and R¹⁰ is H, D; preferably, Y³ is CH.

9. The compound of claim 1, wherein, the compounds of Formula (I) are represented by compounds of Formula (II): 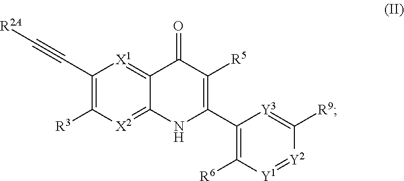
or a pharmaceutically acceptable salt, stereoisomer, solvate, N-oxide, tautomer, isotopic variant, prodrug or deuterated compound thereof;
wherein, X¹, X², Y¹, Y², Y³, R^{2A}, R⁵, R⁶, R³ and R⁹ are defined with respect to Formula (I).

10. The compound of claim 9, wherein, R^{2A} is selected from H, D, CN, C₁-C₆ alkyl, C₂-C₆ alkenyl, C₂-C₆ alkynyl, C₆-C₁₀ aryl, C₃-C₁₀ cycloalkyl, 5-10 membered heteroaryl, 4-10 membered heterocycloalkyl, C(O)R^{B}, C(O)NR^{C}R^{D}, C(O)OR^{A}, S(O)R^{B}, S(O)NR^{C}R^{D}, S(O)₂R^{B}, S(O)₂NR^{C}R^{D}, SiR^{G}R^{H}R^{I}; wherein, the C₁-C₆ alkyl, C₂-C₆ alkenyl, C₂-C₆ alkynyl, C₆-C₁₀ aryl, C₃-C₁₀ cycloalkyl, 5-10 membered heteroaryl, or 4-10 membered heterocycloalkyl is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂;
preferably, R^{2A} is selected from H, D, CN, C₁-C₆ alkyl, C₆-C₁₀ aryl, C₃-C₁₀ cycloalkyl, 5-10 membered heteroaryl, 4-10 membered heterocycloalkyl; wherein each is optionally substituted by 1, 2, 3, 4, or 5 substituents independently selected from D, halo, CN, oxo, C₁-C₄ alkyl, phenyl, C₃-C₆ cycloalkyl, 5-6 membered heteroaryl, 4-6 membered heterocycloalkyl, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, SiR^{g}R^{h}Rⁱ, B(OR^{c})(OR^{d}), C(═NR^{c})NR^{c}R^{d}, NR^{d}C(═NR)NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, S(O)(═NR^{c})R^{b} or NR^{c}S(O)(=NR^{c})R^{b}; wherein, the C₁-C₄ alkyl, phenyl, C₃-C₆ cycloalkyl, 5-6 membered heteroaryl, 4-6 membered heterocycloalkyl is optionally substituted by D, halo, CN, OH, NH₂, C₁-C₄ alkyl, C₁-C₄ haloalkyl, OC₁-C₄ alkyl, OC₁-C₄ haloalkyl, NHC₁-C₄ alkyl, N(C₁-C₄ alkyl)₂; or
preferably, R^{2A} is H, D, CN, CH₃, CH₂CH₃, CH(CH₃)₂,

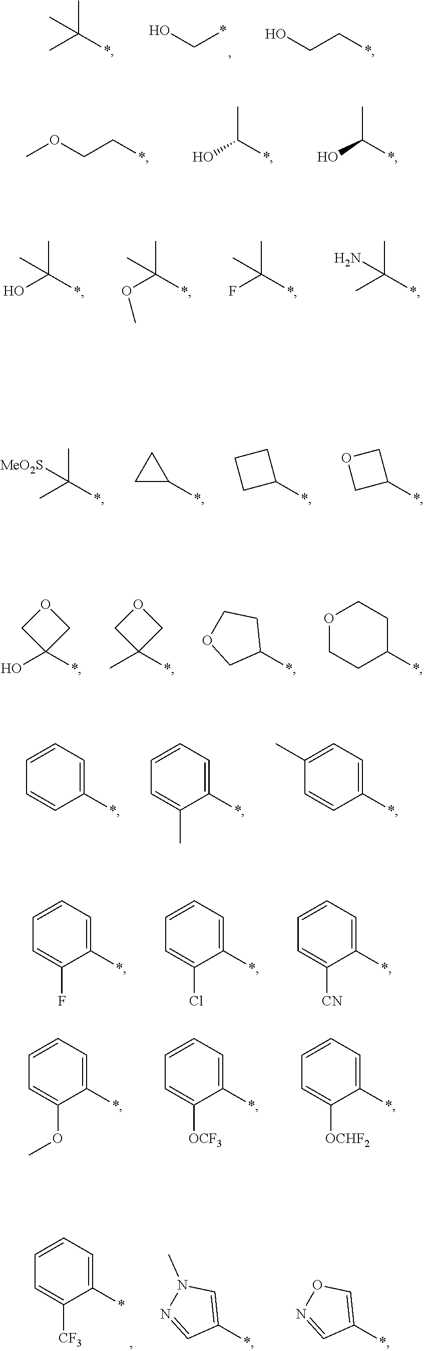

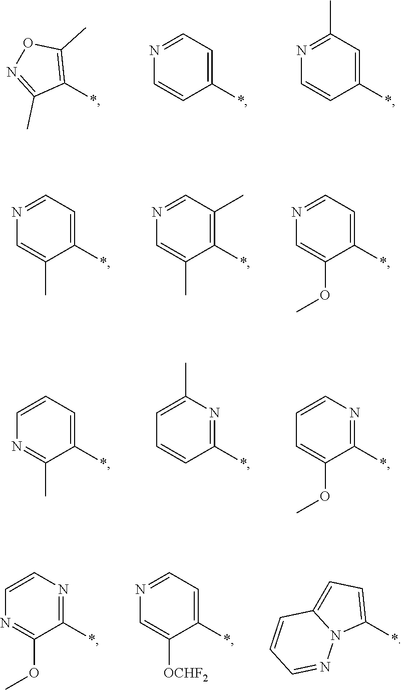

11. The compound of claim 1, wherein, the compound of Formula (I) is: 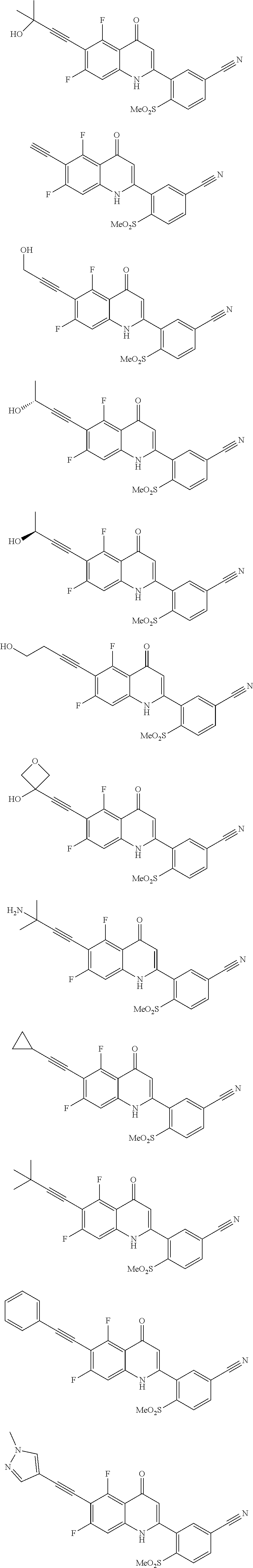 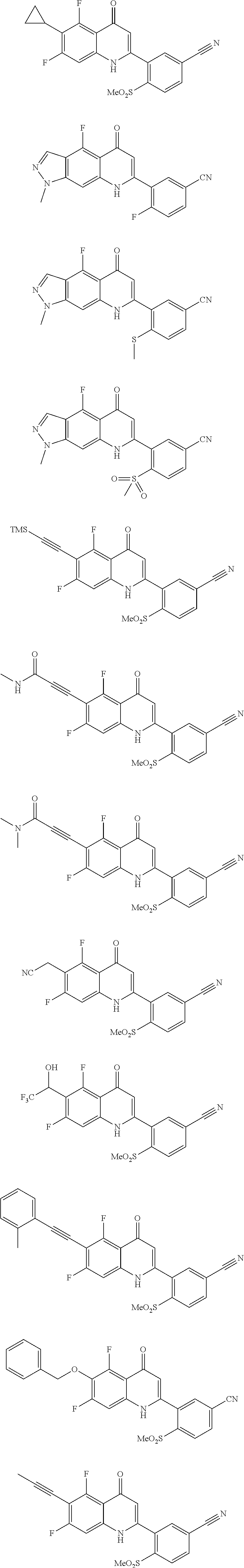 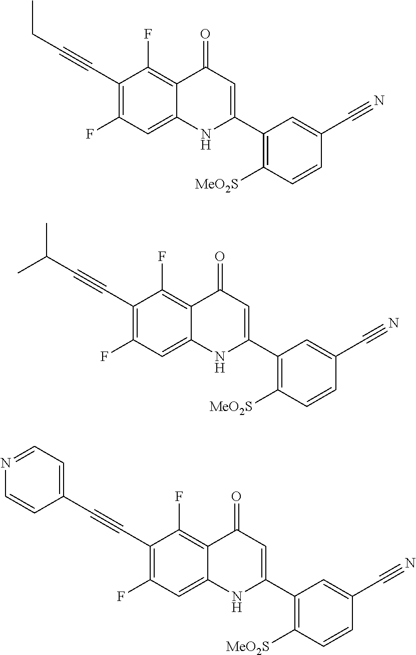 [SMILES: CCC#Cc1c(F)cc2[nH]c(-c3cc(C#N)ccc3S(C)(=O)=O)cc(=O)c2c1F ; CC(C)C#Cc1c(F)cc2[nH]c(-c3cc(C#N)ccc3S(C)(=O)=O)cc(=O)c2c1F ; CS(=O)(=O)c1ccc(C#N)cc1-c1cc(=O)c2c(F)c(C#Cc3ccncc3)c(F)cc2[nH]1] 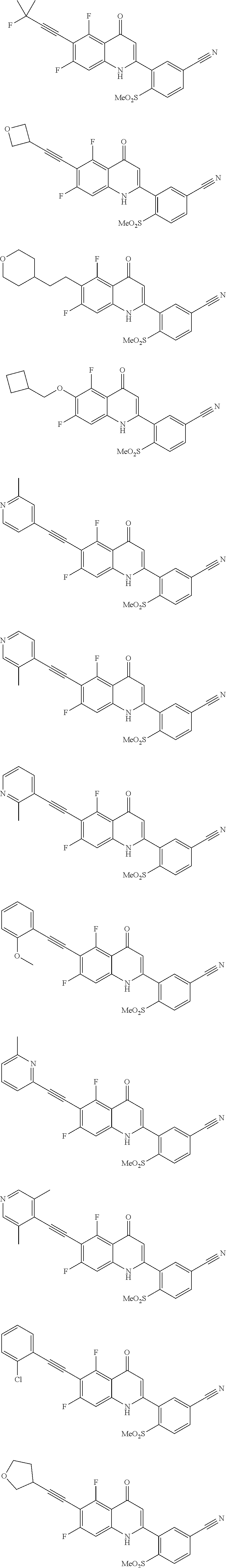 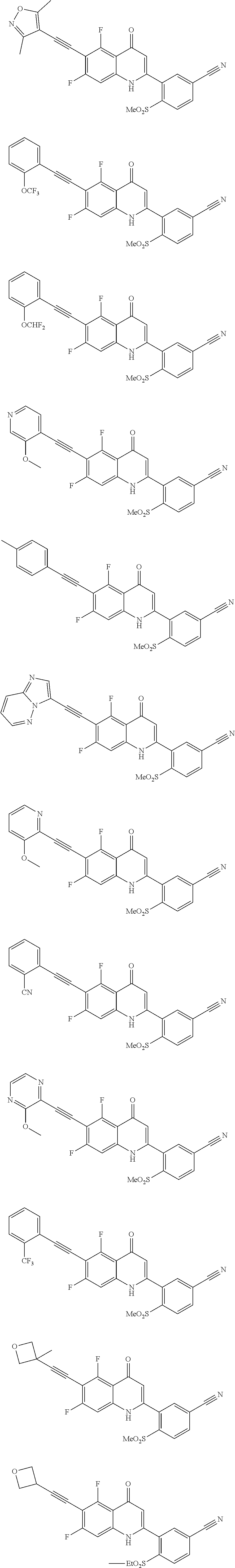 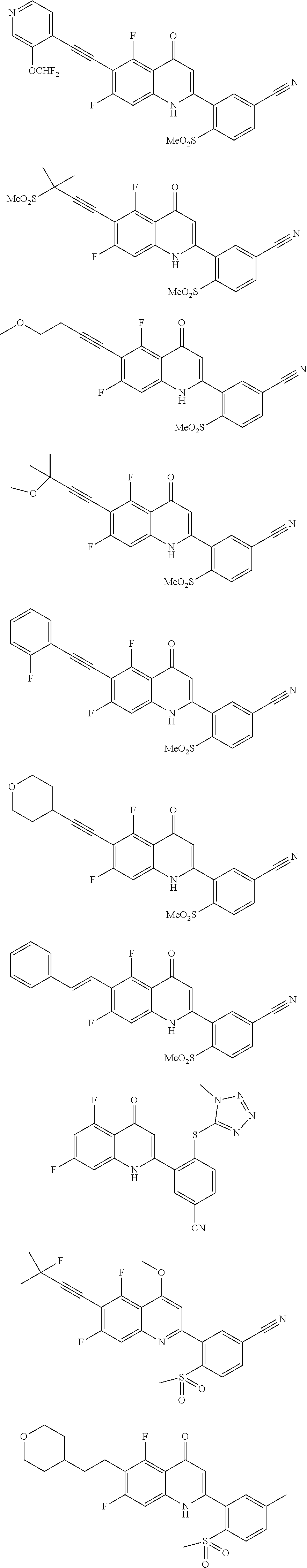
or a pharmaceutically acceptable salt thereof.

12. A pharmaceutical composition comprising the compound of claim 1, or a pharmaceutically acceptable salt, stereoisomer, solvate, N-oxide, tautomer, isotopic variant, prodrug, or deuterated compound thereof, and at least one pharmaceutically acceptable carrier or excipient.

13. A method of modulating PPARγ in cell, which is associated with activated function of PPARγ (gain-of-function mutation, amplification or overexpression) or RXRα gain-of-function mutation (such as S427F/Y), comprising: contacting the cell with the compound of claim 1, or a pharmaceutically acceptable salt or a pharmaceutically acceptable salt, stereoisomer, solvate, N-oxide, tautomer, isotopic variant, prodrug, or deuterated compound.

14. A method of treating a subject in need thereof comprising administering to the subject the compound of claim 1, or a pharmaceutically acceptable salt, stereoisomer, solvate, N-oxide, tautomer, isotopic variant, prodrug, or deuterated compound;
preferably, the subject is suffering from, and is in need of a treatment for, a disease or disorder having the symptom of PPARγ amplification, overexpression and gain-of-function mutation or RXRα gain-of-function mutation; more preferably, the disease or disorder is a cancer; even more preferably, the cancer is a cancer with activated function of PPARγ (gain-of-function mutation, amplification or overexpression) or RXRα gain-of-function mutation (such as S427F/Y).

15. The method of claim 14, wherein, the cancer is Bladder cancer.

16. The method of claim 14, wherein, the subject is human.
